(19) United States
(12) Patent Application Publication
(10) Pub. No.: US 2019/0077856 A1
(43) Pub. Date: Mar. 14, 2019

(54) METHOD OF TREATING DISEASES USING KINASE MODULATORS

(71) Applicant: Memorial Sloan Kettering Cancer Center, New York, NY (US)

(72) Inventors: David A. Scheinberg, New York, NY (US); Elliot Joseph Brea, New York, NY (US); Claire Y. Oh, Colonia, NJ (US); Eusebio Manchado-Robles, Basel (CH); Ralph James Garippa, Roseland, NJ (US)

(73) Assignee: Memorial Sloan Kettering Cancer Center, New York, NY (US)

(21) Appl. No.: 16/084,720

(22) PCT Filed: Mar. 13, 2017
(86) PCT No.: PCT/US2017/022099

§ 371 Date: Sep. 13, 2018

Related U.S. Application Data
(60) Provisional application No. 62/308,859, filed on Mar. 15, 2016.

(51) Int. Cl. C07K 16/22; A61P 35/00; A61K 31/506; A61K 31/519; A61P 37/06; C12N 15/117; C12N 5/0784; A61K 31/5377; A61K 31/517

(52) U.S. Cl. CPC C07K 16/22; A61P 35/00; A61K 31/506; A61K 31/519; A61P 37/06; C12N 2501/727; C12N 5/0639; A61K 31/5377; A61K 31/517; C12N 2310/17; C12N 15/117

(57) ABSTRACT

Provided herein are methods of modulating immune response, including methods of treating a cancer or an infection using a combination of kinase modulators and immunotherapy that promotes immune response. Also provided herein are methods of treating an autoimmune disease or graft-versus-host disease, and methods of reducing the risk of solid organ transplant rejection using a combination of kinase modulators and immunosuppressive therapy.

### CROSS-REFERENCE TO RELATED APPLICATIONS

This application claims the benefit of U.S. Provisional Patent Application No. 62/308,859, filed Mar. 15, 2016, which is incorporated by reference herein in its entirety. National Institutes of Health. The government has certain rights in the invention.

### GOVERNMENT RIGHTS STATEMENT

This invention was made with government support under CA055349 awarded by

### 1. FIELD

Provided herein are methods of modulating immune response, including methods of treating a cancer or an infection using a combination of kinase modulators and immunotherapy that promotes immune response. Also provided herein are methods of treating an autoimmune disease or graft-versus-host disease, and methods of reducing the risk of solid organ transplant rejection using a combination of kinase modulators and immunosuppressive therapy.

### 2. BACKGROUND

Major histocompatibility complex class I molecules (MHC-I) generally present short peptides from either foreign or native intracellular proteins on the cell surface in an HLA-restricted manner for recognition by CD8⁺ T cells via their T cell receptor (TCR) (Agrawal and Kishore, 2000, J Hematother Stem Cell Res 9:795-812). MHC-I is an essential protein for CD8⁺ cytotoxic T cell responses, effective vaccination, adoptive T cell therapies, hematopoietic stem cell transplantation, and organ rejection, among many important physiologic processes and therapeutic manipulations. In addition, the therapeutic TCR-mimic antibodies are directed to MHC/peptide complexes (Dao et al., 2013, Sci Transl Med 5:176ra33; Birnbaum et al., 2014, Cell 157:1073-1087).

While immunotherapies for cancer, infectious disease, and autoimmune disease continue to gain use as effective therapeutic strategies, the mechanisms underlying the control of presentation of foreign antigens or self-tumor antigens are only partially understood and currently not exploited clinically (Pardoll, 2012, Nat Rev Cancer 12:252-264). Reduced cell surface presentation of tumor antigens on MHC-I is an important obstacle to effective immunotherapy with adoptively transferred T-cells, TCR constructs, tumor vaccines, and TCR-mimic antibodies (Mellman et al., 2011, Nature 480:480-489). Moreover, immunotherapies, such as the CTLA-4 blocking antibody tremelimumab, that rely on antigen presentation on MHC-I are being tested in mesothelioma (Calabro et al., 2013, Lancet Oncol 14:1104-1111).

Citation of a reference herein shall not be construed as an admission that such is prior art to the present disclosure.

### 3. SUMMARY OF THE INVENTION

The present invention provides methods of treating cancers or infections using a combination of kinase modulators and immunotherapy that promotes immune response. Also provided herein are methods of treating autoimmune diseases or graft-versus-host diseases, and methods of reducing the risk of solid organ transplant rejection using a combination of kinase modulators and immunosuppressive therapy.

In one aspect, provided herein are methods of treating a cancer in a patient comprising: (i) administering to the patient an inhibitor of the activity of a kinase selected from the group consisting of GRK7 (G Protein-Coupled Receptor Kinase 7), EGFR (Epidermal Growth Factor Receptor), RET (Ret Proto-Oncogene), and BRSK1 (BR Serine/Threonine Kinase 1), and (ii) administering to the patient an immunotherapy that promotes an immune response against the cancer. In various embodiments, the inhibitor is administered in a subclinical amount.

In another aspect, provided herein are methods of generating a population of antigen-presenting cells for therapeutic administration to a patient having a cancer, comprising culturing antigen-presenting cells that are loaded with or genetically engineered to express one or more immunogenic peptides or proteins derived from one or more antigens of the cancer in the presence of an inhibitor of the activity of a kinase selected from the group consisting of GRK7, EGFR, RET, and BRSK1. In another aspect, provided herein are methods of treating a cancer in a patient comprising generating a population of antigen-presenting cells according to such a method and administering to the patient the population of antigen-presenting.

In another aspect, provided herein are methods of treating a cancer in a patient comprising: (i) administering to the patient an activator of the activity of a kinase selected from the group consisting of DDR2 (Discoidin Domain Receptor Tyrosine Kinase 2), CDK7 (Cyclin-Dependent Kinase 7), MINK1 (Misshapen-Like Kinase 1), DAPK3 (Death-Associated Protein Kinase 3), and MAPK3 (Mitogen-Activated Protein Kinase 3), and (ii) administering to the patient an immunotherapy that promotes an immune response against the cancer. In various embodiments, the activator is administered in a subclinical amount.

In another aspect, provided herein are methods of generating a population of antigen-presenting cells for therapeutic administration to a patient having a cancer, comprising culturing antigen-presenting cells that are loaded with or genetically engineered to express one or more immunogenic peptides or proteins derived from one or more antigens of the cancer in the presence of an activator of the activity of a kinase selected from the group consisting of DDR2, CDK7, MINK1, DAPK3, and MAPK3. In another aspect, provided herein are methods of treating a cancer in a patient comprising generating a population of antigen-presenting cells according to such a method and administering to the patient the population of antigen-presenting cells.

A solid tumor cancer that can be treated in accordance with the methods described in this disclosure can be, but is not limited to: breast cancer, lung cancer, ovary cancer, stomach cancer, pancreatic cancer, larynx cancer, esophageal cancer, testes cancer, liver cancer, parotid cancer, biliary tract cancer, colon cancer, rectum cancer, cervix cancer, uterus cancer, endometrium cancer, renal cancer, bladder cancer, prostate cancer, thyroid cancer, melanoma, or non-small cell lung cancer. In a specific embodiment, the cancer is lung cancer (e.g., non-small cell lung cancer), thyroid cancer, or melanoma.

In another aspect, provided herein are methods of treating an infection in a patient comprising: (i) administering to the patient an inhibitor of the activity of a kinase selected from the group consisting of GRK7, EGFR, RET, and BRSK1, and (ii) administering to the patient an immunotherapy that promotes an immune response against the infection. In various embodiments, the inhibitor is administered in a subclinical amount.

In another aspect, provided herein are methods of generating a population of antigen-presenting cells for therapeutic administration to a patient having an infection, comprising culturing antigen-presenting cells that are loaded with or genetically engineered to express one or more immunogenic peptides or proteins derived from one or more antigens of the pathogen causing the infection in the presence of an inhibitor of the activity of a kinase selected from the group consisting of GRK7, EGFR, RET, and BRSK1. In another aspect, provided herein are methods of treating an infection in a patient comprising generating a population of antigen-presenting cells according to such a method and administering to the patient the population of antigen-presenting cells.

In another aspect, provided herein are methods of treating an infection in a patient comprising: (i) administering to the patient an activator of the activity of a kinase selected from the group consisting of DDR2, CDK7, MINK1, DAPK3, and MAPK3, and (ii) administering to the patient an immunotherapy that promotes an immune response against the infection. In various embodiments, the activator is administered in a subclinical amount.

In another aspect, provided herein are methods of generating a population of antigen-presenting cells for therapeutic administration to a patient having an infection, comprising culturing antigen-presenting cells that are loaded with or genetically engineered to express one or more immunogenic peptides or proteins derived from one or more antigens of the pathogen causing the infection in the presence of an activator of the activity of a kinase selected from the group consisting of DDR2, CDK7, MINK1, DAPK3, and MAPK3. In another aspect, provided herein are methods of treating an infection in a patient comprising generating a population of antigen-presenting cells according to such a method and administering to the patient the population of antigen-presenting cells.

In certain embodiments, the infection to be treated is an infection with a virus, bacterium, fungus, helminth or protist. In specific embodiments, the infection is an infection with a virus. In a specific embodiment, the infection is an infection with herpesvirus. In another specific embodiment, the infection is an infection with cytomegalovirus.

In another aspect, provided herein are methods of treating an autoimmune disease in a patient comprising: (i) administering to the patient an activator of the activity of a kinase selected from the group consisting of GRK7, EGFR, RET, and BRSK1, and optionally (ii) administering to the patient an immunosuppressive therapy that suppresses the immune response associated with the autoimmune disease. In various embodiments, the activator is administered in a subclinical amount.

In another aspect, provided herein are methods of treating an autoimmune disease in a patient comprising: (i) administering to the patient an inhibitor of the activity of a kinase selected from the group consisting of DDR2, CDK7, MINK1, DAPK3, and MAPK3, and optionally (ii) administering to the patient an immunosuppressive therapy that suppresses the immune response associated with the autoimmune disease. In various embodiments, the inhibitor is administered in a subclinical amount.

In a specific embodiment, the autoimmune disease is multiple sclerosis, type 1 diabetes, ankylosing spondylitis, or Hashimoto's thyroiditis.

In another aspect, provided herein are methods of treating graft-versus-host disease (GvHD) in a patient comprising: (i) administering to the patient an activator of the activity of a kinase selected from the group consisting of GRK7, EGFR, RET, and BRSK1, and optionally (ii) administering to the patient an immunosuppressive therapy that suppresses the immune response associated with the GvHD. In various embodiments, the activator is administered in a subclinical amount.

In another aspect, provided herein are methods of treating a GvHD in a patient comprising: (i) administering to the patient an inhibitor of the activity of a kinase selected from the group consisting of DDR2, CDK7, MINK1, DAPK3, and MAPK3, and optionally (ii) administering to the patient an immunosuppressive therapy that suppresses the immune response associated with the GvHD. In various embodiments, the inhibitor is administered in a subclinical amount.

In some embodiments, the GvHD to be treated is an acute GvHD. In other embodiments, the GvHD to be treated is a chronic GvHD.

In another aspect, provided herein are methods of reducing the risk of (e.g., prevention of) solid organ transplant rejection in a patient comprising: (i) administering to the patient an activator of the activity of a kinase selected from the group consisting of GRK7, EGFR, RET, and BRSK1, and optionally (ii) administering to the patient an immunosuppressive therapy that suppresses the immune response against the solid organ transplant. In various embodiments, the activator is administered in a subclinical amount.

In another aspect, provided herein are methods of reducing the risk of (e.g., prevention of) solid organ transplant rejection in a patient comprising: (i) administering to the patient an inhibitor of the activity of a kinase selected from the group consisting of DDR2, CDK7, MINK1, DAPK3, and MAPK3, and optionally (ii) administering to the patient an immunosuppressive therapy that suppresses the immune response against the solid organ transplant. In various embodiments, the inhibitor is administered in a subclinical amount.

In specific embodiments, the solid organ transplant is a kidney transplant, a liver transplant, a heart transplant, an intestinal transplant, a pancreas transplant, a lung transplant, a small bowel transplant, a thymus transplant, or a combination thereof

In some embodiments, the inhibitor of the activity of a kinase selected from the group consisting of GRK7, EGFR, RET, and BRSK1, is a small molecule inhibitor. In other embodiments, the inhibitor is an antibody or an antigen-binding fragment thereof that specifically binds to the kinase. In a specific embodiment, the antibody is a monoclonal antibody. In a specific embodiment, the kinase is EGFR and the inhibitor is erlotinib, gefitinib, afatanib, or lapatinib. In another specific embodiment, the kinase is RET and the inhibitor is regorafenib, danusertib, cabozantinib, or AST487 (1-[4-[(4-ethylpiperazin-1-yl)methyl]-3-(trifluoromethyl)phenyl]-3-[4-[6-(methylamino)pyrimidin-4-yl]oxyphenyl]urea).

In some embodiments, the activator of the activity of a kinase selected from the group consisting of DDR2, CDK7, MINK1, DAPK3, and MAPK3, is a soluble ligand of the kinase (e.g., where the kinase is a receptor), or a soluble ligand of a receptor that activates the kinase in vivo. In other embodiments, the activator is an antibody or an antigen-binding fragment thereof that specifically binds to the kinase. In a specific embodiment, the antibody is a monoclonal antibody.

In some embodiments, the activator of the activity of a kinase selected from the group consisting of GRK7, EGFR, RET, and BRSK1, is a soluble ligand of the kinase (e.g., where the kinase is a receptor), or a soluble ligand of a receptor that activates the kinase in vivo. In other embodiments, the activator is an antibody or an antigen-binding fragment thereof that specifically binds to the kinase. In a specific embodiment, the antibody is a monoclonal antibody.

In some embodiments, the inhibitor of the activity of a kinase selected from the group consisting of DDR2, CDK7, MINK1, DAPK3, and MAPK3, is a small molecule inhibitor. In other embodiments, the inhibitor is an antibody or an antigen-binding fragment thereof that specifically binds to the kinase. In a specific embodiment, the antibody is a monoclonal antibody. In a specific embodiment, the kinase is DDR2 and the inhibitor is dasatinib. In another specific embodiment, the kinase is CDK7 and the inhibitor is BS-181 HCl (N5-(6-aminohexyl)-N7-benzyl-3-isopropylpyrazolo[1,5-a]pyrimidine-5,7-diamine hydrochloride). In another specific embodiment, the kinase is DAPK3 and the inhibitor is 324788 ((4Z)-4-(3-Pyridylmethylene)-2-styryl-oxazol-5-one). In another specific embodiment, the kinase is MAPK3 and the inhibitor is ulixertinib.

In some embodiments, the immunotherapy that promotes an immune response is a vaccine.

In other embodiments, the immunotherapy that promotes an immune response is an immune checkpoint blockade. In specific embodiments, the immune checkpoint blockade is an antibody or an antigen-binding fragment thereof that specifically binds to and reduces the activity of an immune checkpoint protein. In a specific embodiment, the antibody is a monoclonal antibody. In certain embodiments, the immune checkpoint blockade inhibits the activity of CTLA-4, PD-1, PD-L1, PD-L2, TIM-3, or LAG-3.

In other embodiments, the immunotherapy that promotes an immune response is an adoptive immunotherapy, such as an adoptive T cell therapy. In a specific embodiment, the adoptive T cell therapy is TCR (T-Cell Receptor)-engineered T cells. In another specific embodiment, the adoptive T cell therapy is CAR T cells, wherein the antigen-binding domain of the CAR (Chimeric Antigen Receptor) specifically binds to an antigen of the cancer.

In other embodiments, the immunotherapy that promotes an immune response is a TCR mimic antibody.

In other embodiments, the immunotherapy that promotes an immune response is a TCR based construct that encodes a soluble protein comprising the antigen recognition domain of a TCR.

In other embodiments, the immunotherapy that promotes an immune response is an interferon (preferably interferon alpha or interferon gamma), an anti-CD47 antibody, a SIRP alpha antagonist, an HDAC inhibitor, a cytokine, a TLR agonist, or an epigenetic modulator that upregulates the expression of one or more MHCs (Major Histocompatibility Complexes) or upregulates antigen presentation. In a specific embodiment, the immunotherapy that promotes an immune response is an epigenetic modulator that upregulates the expression of one or more MHCs or upregulates antigen presentation that is a hypomethylating agent (e.g., azacytidine or decitabine). In another specific embodiment, the immunotherapy that promotes an immune response is an interferon that is interferon alpha or interferon gamma. In another specific embodiment, the immunotherapy that promotes an immune response is a cytokine that is IL2 (Interleukin-2), TNF (Tumor Necrosis Factor), interferon alpha or interferon gamma. In another specific embodiment, the immunotherapy that promotes an immune response is a TLR agonist that is a dsDNA (double-stranded DNA) TLR agonist. In another specific embodiment, the immunotherapy that promotes an immune response is a TLR agonist that is a dsRNA (double-stranded RNA) TLR agonist (e.g., polyinosinic-polycytidylic acid (poly(I:C)).

In various embodiments, the immunosuppressive therapy can be sirolimus, everolimus, rapamycin, one or more steroids, cyclosporine, cyclophosphamide, azathioprine, mercaptopurine, fluorouracil, fludarabine, interferon beta, a TNF decoy receptor, a TNF antibody, methotrexate, a T-cell antibody, an anti-CD20 antibody, a complement inhibitor, an anti-IL6 (Interleukin-6) antibody, an anti-IL2R (Interleukin-2 Receptor) antibody, anti-thymocyte globulin, fingolimod, mycophenolate, or a combination thereof.

In some embodiments, the immunosuppressive therapy is a TNF decoy receptor (e.g., etanercept). In other embodiments, the immunosuppressive therapy is a TNF antibody (e.g., infliximab). In other embodiments, the immunosuppressive therapy is a T-cell antibody (e.g., an anti-CD3 antibody, such as OKT3). In other embodiments, the immunosuppressive therapy is an anti-CD20 antibody (e.g., rituximab). In other embodiments, the immunosuppressive therapy is a complement inhibitor (e.g., eculizumab). In other embodiments, the immunosuppressive therapy is an anti-IL2R antibody (e.g., daclizumab).

In a preferred embodiment, the patient is a human patient.

### 4. BRIEF DESCRIPTION OF FIGURES

FIG. 1A-FIG. 1E. Screen for kinase regulators of surface HLA. FIG. 1A: A TRMPV inducible short hairpin RNA (shRNA) retroviral vector was used for transducing JMN (HLA-A*02:01 positive human mesothelioma line) cells. TRE is the Tet responsive element, which drives expression of the fluorophore dsRed and the shRNA hairpin. The constitutive PGK promoter drives the Venus fluorophore along with Neomycin resistance (NeoR) cassette. FIG. 1B: Western blot (left) showing and flow cytometry data (right) showing knockdown of HLA-A using TRMPV retroviral system with a positive control shRNA to HLA-A02. The shRen is a negative control shRNA designed against the Renilla gene. FIG. 1C: Schema depicting the selection criteria plan for the screen of regulators of surface HLA-A. FIG. 1D: Waterfall plot showing distribution of shRNA constructs against mitogen-activated protein kinase kinase (MAP2K1) and epidermal growth factor receptor (EGFR) as log fold difference between BB7 high sorted population and BB7 low sorted population. FIG. 1E: shRNA knockdown of MAP2K1 and EGFR in JMN cells validated them as a negative regulator of surface HLA-A. BB7.2 (i.e., BB7) is a mAb specific for HLA-A02. shRNA against Renilla was used as a negative control, while an shRNA against HLA-A was used as a positive control. Student's t-test was done to compare each shRNA gene knockdown mean fluorescence intensity (MFI) to the shRen control. (*≤0.05, **≤0.01, ***≤0.001, ****≤0.0001),

FIG. 2A-FIG. 2H: Use of selective EGFR inhibitor (EGFRi) and mitogen-activated protein kinase kinase 1 (MEK) inhibitor (MEKi), increased cell surface HLA-A expression, and tumor antigen presentation, while activation of EGFR caused downregulation of MHC-I. FIG. 2A: MEK inhibition and EGFR inhibition for 72 hours with the indicated inhibitors increased HLA-A (BB7 binding) by flow cytometry in JMN, Meso34, PC-9, UACC257, SK-MEL-5, SW480, CFPAC-1 and TPC1 cell lines. 1% DMSO was used as a vehicle control. FIG. 2B: Binding of TCRm (TCR mimic) antibodies to peptide /MHC epitopes. Use of ESK antibody to a peptide derived from the oncoprotein WT1 that is presented on HLA-A0201. Binding increased after inhibition of EGFR and MEK for 72 hours in JMN, Meso34, and TPC1. PRAME is a TCRm antibody against an epitope of PRAME tumor antigen presented on HLA-A0201 on SKMEL5. Experimental setup was similar to FIG. 2A. FIG. 2C: Treatment of JMN with 10 nM EGF for 72 hours, causing activation of the downstream MAPK pathway, led to decreased surface HLA-A and total HLA-ABC. FIG. 2D: Use of EGFRi erlotinib and afatanib, along with MEKi trametinib on H827 (EGFR E746del-A750 mutation), H1975 (L858R/T790M), H1299 (EGFR wt, NRAS Q61K), and A549 (EGFR wt/KRAS G12S) to alter surface HLA-ABC levels. Student's t-test was done to compare each treatment to vehicle control. *P values annotated as in FIG. 1E. FIG. 2E: Western blot analysis showing level of inhibition of the MAP kinase pathway on panel of NSCLC cell lines using 1% dimethyl sulfoxide (DMSO) (D), 100 nM erlotinib (E), 100 nM afatanib (A), or 500 nM trametinib (T). FIG. 2F: H1299 cells were transduced with retroviral vectors expressing EGFR L858R and were analyzed for surface pan HLA-ABC using W6/32. Activation of EGFR was demonstrated by western blot. FIG. 2G: EGFR inhibition upregulated surface HLA-ABC more than MEKi despite equivalent levels of inhibition of phospho-extracellular signal-regulated kinase (pERK) output. FIG. 2H: EGFRi up-regulated MHC-I despite downstream mutations causing constitutive MAPK activation. The NRAS Q61K mutation was introduced into H827 and cells were treated with EGFRi or MEKi as done in FIG. 2G.

FIG. 3A-FIG. 3D: Improving immunotherapy efficacy by up-regulating cell surface HLA-A. FIG. 3A: Antibody dependent cellular cytotoxicity (ADCC) assay was performed on JMN human mesothelioma cell line. Cells were incubated for 72 hours with either vehicle control or trametinib and subsequently exposed to either isotype antibody or ESKM in ADCC assay. FIG. 3B depicts an ADCC assay on Meso34 (human mesothelioma). The experimental setup was similar to FIG. 3A. FIG. 3C depicts an ADCC assay on SKMEL5 (human melanoma) using TCRm mAb PRAME against the PRAME epitope; the experimental setup was similar to FIG. 3A. FIG. 3D: B16F10 cells were exposed to pmel-1 (gp100) specific TCR T-cells for 24 hours, then killing was assessed using a clonogenic assay described previously (Budhu et al. 2010, J Exp Med 207:223-235)

FIG. 4A-FIG. 4G: MAPK signaling suppressed antigen presentation machinery and MAPK inhibition broadly up-regulated antigen presentation machinery. FIG. 4A: MEK and EGFR inhibition for 48 hours led to increased levels of HLA-A, along with antigen peptide transporter 1 (TAP1), antigen peptide transporter 2 (TAP2), and beta-2-microglobulin (B2M) in JMN, Meso34, SK-MEL-5 and UACC257, H827, and PC9. FIG. 4B: Dose dependent increase in surface HLA-A with increasing MEKi in JMN and SKMEL5 Cells were analyzed by flow cytometry at 72 hours. FIG. 4C: MEK inhibition leads to increasing levels of HLA-A and B2M protein. Cells were treated with indicating amounts of trametinib (MEKi) for 72 hours and specific antibodies against indicated proteins were blotted. FIG. 4D: Overexpression of B2M leads to increased surface HLA-A and HLA-ABC. FIG. 4E: Treatment of JMN with trametinib for 72 hours led to increased activity on the HLA-A and B2M promoter. The HLA-A and B2M promoter was cloned upstream of the Gaussian Luciferase (GLuc) gene. Secreted embryonic alkaline phosphatase (SEAP) under the CMV promoter was used as a normalization factor. FIG. 4F: Knockdown of signal transducer and activator of transcription 1 (STAT1), on JMN cells treated with MEKi demonstrates role in mediating surface HLA-A up-regulation. JMN cells were transfected with small interfering RNA (siRNA) against genes shown and treated with either DMSO or 1 uM trametinib 24 hours after siRNA transfection, then assayed by flow cytometry for surface HLA-A expression 72 hours after treatment. FIG. 4G: Unsupervised hierarchical clustering microarray expression profiling analysis of lung tumors from CC10/L858R mice with EGFR L858R tumor bearing lungs (four right columns) or normal lungs (the five left columns) focusing on H2-KD (H-2 class I histocompatibility antigen), B2M, TAP1, TAP2, PD-L1 (programmed cell death ligand 1, also referred to as CD274), and PD1 (programmed cell death 1, also referred to as PDCD1) gene expression.

FIG. 5: Knockdown of HLA-A caused resistance to antibody dependent cellular cytotoxicity by the ESK-M monoclonal antibody (mAb). JMN transduced with either control shRen or shHLA-A and induced for 96 hours was used for in vitro ADCC assay. Isotype or ESK-M was used as described in Section 6.1.

FIG. 6: Coordinated regulation of total surface HLA-A, B, and C with knockdown of either MAP2K1 of EGFR. Flow cytometry data showing knockdown of MAP2K1 or EGFR, compared to control, led to increased pan HLA-ABC. The experimental setup was similar to FIG. lE but using W6/32 (Pan HLA-ABC mAb).

FIG. 7: Validation of MAP2K1, EGFR, and ret proto-oncogene (RET) as negative kinase regulators in mesothelioma cell lines. FIG. 7A: Flow cytometry showing knockdown of MAP2K1 validated MAP2K1 as a negative regulator of surface HLA-A in mesothelioma cell lines. FIG. 7B: Flow cytometry showing knockdown of EGFR validated EGFR as a negative regulator of surface HLA-A in mesothelioma cell line Meso56. FIG. 7C: Flow cytometry showing knockdown of RET validated RET as a negative regulator of surface HLA-A in mesothelioma cell lines

FIG. 8A-FIG. 8C: DDR2 (discoidin domain receptor tyrosine kinase 2) and MINK1 (misshapen-like kinase 1) acted as positive regulators of surface HLA-A. FIG. 8A: Waterfall plot showing fold difference between BB7 high and low sorted population and distribution of shRNA constructs against DDR2. FIG. 8B: Waterfall plot showing fold difference between BB7 high and low sorted population and distribution of shRNA constructs against MINK1. FIG. 8C: DDR2 and MINK1 knockdown decreased surface HLA-A expression in JMN. Cells were transfected with siRNA against either a scrambled siRNA, an siRNA against HLA-A, or an siRNA against DDR2 or MINK1. After 96 hours, flow cytometry was performed. Statistical significance was compared to scrambled siRNA using unpaired student T-test on triplicate samples.

FIG. 9A-FIG. 9B: Viability of JMN after treatment with MEKi trametinib and EGFRi Afatanib. Cell Titer Glo (Promega) was performed on JMN using trametinib (FIG. 9A) and afatanib (FIG. 9B) at indicated concentrations. Cells were incubated with inhibitors for 48 hours before performing Cell Titer glo assay.

FIG. 10A-FIG. 10C: Titration of trametinib to determine optimal inhibition of MEK using pERK as a marker of inhibition. FIG. 10A: JMN and Meso34 were incubated with increasing doses of trametinib for 1 hour and analyzed by western blot for pERK and ERK. FIG. 10B: Cells were incubated with trametinib for 72 hours. FIG. 10C: SKMEL5 and UACC257 were analyzed by western blot at 72 hours for pERK and ERK.

FIG. 11: JMN was treated with 50 nM trametinib and lysate was analyzed as in FIG. 4C at different time points.

FIG. 12: EGFR inhibition led to increasing levels of HLA-A and B2M protein. Cells were treated with indicating amounts of erlotinib (EGFRi) for 72 hours and specific antibodies against indicated proteins were blotted.

FIG. 13A-FIG. 13D: MSK Memorial Hospital IMPACT genomic sequencing data for the JMN human mesothelioma cell line. FIG. 13A: Non-synonymous mutations in JMN using the IMPACT-410 platform. FIG. 13B: Copy number alterations using the IMPACT-410 platform. FIG. 13C: IMPACT-410 panel on Meso34 human mesothelioma cell line. FIG. 13D: Copy number alterations using IMPACT-410 on Meso34.

FIG. 14A-FIG. 14C: TPC1 (a papillary thyroid cancer cell line) treated with AST487 increased HLA surface expression. FIG. 14A and FIG. 14B: TPC1 cells were seeded and treated with different doses of AST487 (a RET inhibitor). After 72 hours, cells were harvested and surface HLA-A02 and HLA-ABC were measured through flow cytometry with BB7 and W6/32 staining antibodies, respectively. FIG. 14C: After 24 hours of AST487 incubation, cells were lysed and a western blot was performed to show a decrease in pRET and pERK with AST487 treatment.

FIG. 15A-FIG. 15C: TT cells (a medullary thyroid cancer cell line) also upregulated HLA-A with AST487 treatment. FIG. 15A and FIG. 15B: TT cells were seeded and treated with different doses of AST487 (a RET inhibitor). After 72 hours, cells were harvested and surface HLA-A02 and HLA-ABC were measured through flow cytometry with BB7 and W6/32 staining antibodies, respectively. FIG. 15C: After 24 hours of AST487 incubation, cells were lysed and a western blot was performed. TT cells are derived from a medullary thyroid cancer that has a RET point mutation, as opposed to the TPC1 cells, which are derived from a papillary thyroid cancer and have a RET/PTC1 fusion.

FIG. 16A-FIG. 16B: Validating RET as a regulator of HLA. To validate that RET regulated HLA, siRNAs and another small molecule inhibitor that targeted RET were used. FIG. 16A: TPC1 cells were treated with siRNAs against a scrambled gene or the RET gene for 96 hours. At that time, surface HLA-A02 and HLA-ABC were measured with BB7 and W6/32 staining antibodies. FIG. 16B: TPC1 cells were incubated with cabozantinib (a small molecule inhibitor of tyrosine kinases met proto-oncogene (c-MET), vascular endothelial growth factor 2 (VEGF2), KIT proto-oncogene receptor tyrosine kinase (c-KIT), fms-related tyrosine kinase 3 (FLT3) and RET) for 72 hours and surface HLA-A02 and HLA-ABC were measured.

FIG. 17: Regulation of HLA was seen at the transcript level. Using qPCR, transcript levels of HLA and antigen processing machinery were measured after TPC1 cells were treated with AST487 for 24 (FIG. 17A) or 48 hours (FIG. 17B). Upregulation of mRNA levels for HLA and antigen processing machinery were seen.

FIG. 18A-FIG. 18B: AST487 increased cytolytic activity of TCRm antibody. FIG. 18A: TPC1 cells were treated with different doses of AST487 and binding of ESK (a TCR mimic monoclonal antibody specific for the WT1 RMF peptide/HLA-A02:01 complex) was measured. FIG. 18B: With increase of ESK binding in vitro, the effect on cytolytic activity of ESK was measured with an ADCC assay. TPC1 cells were treated with AST487 or DMSO for 72 hours and then incubated with chromium. Peripheral blood mononuclear cells (PBMCs), chromium labeled target cells, and ESK-M (or an isotype) were mixed and incubated for 5 hours. Varying effector to target ratios were used. Afterwards, chromium levels in the media were measured to determine percent specific lysis.

FIG. 19A-FIG. 19B: AST487 treatment in vivo increased surface HLA expression levels. FIG. 19A: NRG mice were injected with TPC1 cells and treated with vehicle (control) or AST487. BB7 and W6/32 binding on TPC1 cells were measured, and are shown in FIG. 19A (normalized to vehicle-treated mice) for vehicle-treated, 10 mg/kg AST487-treated, and 35 mg/kg AST487-treated mice. FIG. 19B: AST487 treatment in vivo did not change PD-L1 expression levels. PD-L1 levels were measured as binding to anti-PD-L1 antibody.

### 5. DETAILED DESCRIPTION

The present invention provides methods of regulating processes involving presentation of peptides by class I MHC (in humans, HLA). The present invention provides methods of treating a cancer, an infection, an autoimmune disease, and graft-versus-host disease (GvHD), respectively, using kinase modulators, and methods of reducing the risk of solid organ transplant rejection using kinase modulators. The invention identifies kinases that are negative regulators of class I MHC gene expression, and kinases that are positive regulators of class I MEW gene expression. Inhibitors of the kinases that are negative regulators of class I MEW (in humans, HLA) gene expression, or activators of the kinases that are positive regulators of class I MEW (in humans, HLA) gene expression, can be used, preferably in combination with immune-promoting immunotherapy, to increase an immune response where such is desired, ex vivo, or in vivo (by administration to a patient), e.g., to treat cancer, viral infection, etc. Inhibitors of the kinases that are positive regulators of class I MEW (in humans, HLA) gene expression, or activators of the kinases that are negative regulators of class I MEW (in humans, HLA) gene expression, can be used, preferably in combination with immunosuppressive therapy, to suppress an immune response where such is desired, ex vivo, or in vivo (by administration to a patient), e.g., to treat autoimmune disease, GvHD, or to reduce the risk of solid organ transplant rejection, etc.

Kinases that are negative regulators of class I MHC (in humans, HLA) gene expression include, but are not limited to, GRK7 (G Protein-Coupled Receptor Kinase 7), EGFR (Epidermal Growth Factor Receptor), RET (Ret Proto-Oncogene), BRSK1 (BR Serine/Threonine Kinase 1), and MAP2K1 (Mitogen-Activated Protein Kinase Kinase 1).

Kinases that are positive regulators of class I MEW (in humans, HLA) gene expression include, but are not limited to, DDR2 (Discoidin Domain Receptor Tyrosine Kinase 2), CDK7 (Cyclin-Dependent Kinase 7), MINK1 (Misshapen-Like Kinase 1), DAPK3 (Death-Associated Protein Kinase 3), and MAPK3 (Mitogen-Activated Protein Kinase 3).

It is shown in the Examples herein that combination treatment using an inhibitor of a kinase that negatively regulates class I MHC gene expression and an immunotherapy have synergistic effect in killing cancer cells.

The inhibitors of kinases used in the methods of the invention decreases or blocks the activity of the kinase. The activators of kinases used in the methods of the invention increases or initiates the activity of the kinase.

### 5.1. Treatment of Cancer

In one aspect, provided herein are methods of treating a cancer in a patient comprising: (i) administering to the patient an inhibitor of the activity of a kinase selected from the group consisting of GRK7 (G Protein-Coupled Receptor Kinase 7), EGFR (Epidermal Growth Factor Receptor), RET (Ret Proto-Oncogene), and BRSK1 (BR Serine/Threonine Kinase 1), and (ii) administering to the patient an immunotherapy that promotes an immune response against the cancer. According to the invention, and without intending to be bound by a mechanism, inhibition of the activity of a kinase selected from the group consisting of GRK7, EGFR, RET, and BRSK1 upregulates class I MHC gene expression on cancer cells (in human patients, such inhibition upregulates HLA-A expression, and preferably also upregulates HLA-B expression and HLA-C expression on cancer cells). In various embodiments, the inhibitor is administered in a subclinical amount. A subclinical amount of the inhibitor refers to an amount of the inhibitor at which no clinical effect or less than optimal clinical effect is detected when the inhibitor is administered alone (i.e., not in combination with the immunotherapy). In specific embodiments, the subclinical amount is lower than the amount of the inhibitor commonly used in the standard-of-care therapy for the cancer to be treated. In specific embodiments wherein the inhibitor is FDA (Food and Drug Administration)-approved for treating the cancer, the subclinical amount is lower than the FDA-approved amount for treating the cancer.

In another aspect, provided herein are methods of generating a population of antigen-presenting cells for therapeutic administration to a patient having a cancer, comprising culturing antigen-presenting cells that are loaded with or genetically engineered to express one or more immunogenic peptides or proteins derived from one or more antigens of the cancer in the presence of an inhibitor of the activity of a kinase selected from the group consisting of GRK7, EGFR, RET, and BRSK1. In another aspect, provided herein are methods of treating a cancer in a patient comprising generating a population of antigen-presenting cells according to such a method and administering to the patient the population of antigen-presenting. The antigen-presenting cells can be, for example, dendritic cells, cytokine-activated monocytes, or PBMCs. Preferably, the antigen-presenting cells are dendritic cells. Preferably, the antigen-presenting cells are autologous to the human patient (e.g., dendritic cells autologous to the human patient).

In another ex vivo embodiment, provided herein are methods of generating a population of antigen-specific T cells for therapeutic administration to a patient having a cancer, comprising co-culturing T cells with antigen-presenting cells that are loaded with or genetically engineered to express one or more immunogenic peptides or proteins derived from one or more antigens of the cancer in the presence of an inhibitor of the activity of a kinase selected from the group consisting of GRK7, EGFR, RET, and BRSK1. In another aspect, provided herein are methods of treating a cancer in a patient comprising generating a population of antigen-specific T cells according to such a method and administering to the patient the population of antigen-specific T cells. The antigen-presenting cells can be, for example, dendritic cells, cytokine- activated monocytes, or PBMCs. Preferably, the antigen-presenting cells are dendritic cells. Preferably, the antigen-presenting cells are autologous to the human patient (e.g., dendritic cells autologous to the human patient).

In another aspect, provided herein are methods of treating a cancer in a patient comprising: (i) administering to the patient an activator of the activity of a kinase selected from the group consisting of DDR2 (Discoidin Domain Receptor Tyrosine Kinase 2), CDK7 (Cyclin-Dependent Kinase 7), MINK1 (Misshapen-Like Kinase 1), DAPK3 (Death-Associated Protein Kinase 3), and MAPK3 (Mitogen-Activated Protein Kinase 3), and (ii) administering to the patient an immunotherapy that promotes an immune response against the cancer. According to the invention, and without intending to be bound by a mechanism, activation of the activity of a kinase selected from the group consisting of DDR2, CDK7, MINK1, DAPK3, and MAPK3 upregulates class I MEW gene expression on cancer cells (in human patients, such activation upregulates HLA-A expression, and preferably also upregulates HLA-B expression and HLA-C expression on cancer cells). In various embodiments, the activator is administered in a subclinical amount. A subclinical amount of the activator refers to an amount of the activator at which no clinical effect or less than optimal clinical effect is detected when the inhibitor is administered alone (i.e., not in combination with the immunotherapy). In specific embodiments, the subclinical amount is lower than the amount of the activator commonly used in the standard-of-care therapy for the cancer to be treated. In specific embodiments wherein the activator is FDA -approved for treating the cancer, the subclinical amount is lower than the FDA-approved amount for treating the cancer.

In another aspect, provided herein are methods of generating a population of antigen-presenting cells for therapeutic administration to a patient having a cancer, comprising culturing antigen-presenting cells that are loaded with or genetically engineered to express one or more immunogenic peptides or proteins derived from one or more antigens of the cancer in the presence of an activator of the activity of a kinase selected from the group consisting of DDR2, CDK7, MINK1, DAPK3, and MAPK3. In another aspect, provided herein are methods of treating a cancer in a patient comprising generating a population of antigen-presenting cells according to such a method and administering to the patient the population of antigen-presenting cells. The antigen-presenting cells can be, for example, dendritic cells, cytokine-activated monocytes, or PBMCs. Preferably, the antigen-presenting cells are dendritic cells. Preferably, the antigen-presenting cells are autologous to the human patient (e.g., dendritic cells autologous to the human patient).

In another ex vivo embodiment, provided herein are methods of generating a population of antigen-specific T cells for therapeutic administration to a patient having a cancer, comprising co-culturing T cells with antigen-presenting cells that are loaded with or genetically engineered to express one or more immunogenic peptides or proteins derived from one or more antigens of the cancer in the presence of an activator of the activity of a kinase selected from the group consisting of DDR2, CDK7, MINK1, DAPK3, and MAPK3. In another aspect, provided herein are methods of treating a cancer in a patient comprising generating a population of antigen-specific T cells according to such a method and administering to the patient the population of antigen-specific T cells. The antigen-presenting cells can be, for example, dendritic cells, cytokine-activated monocytes, or PBMCs. Preferably, the antigen-presenting cells are dendritic cells. Preferably, the antigen-presenting cells are autologous to the human patient (e.g., dendritic cells autologous to the human patient).

While the methods of treating a cancer described in this disclosure are largely methods of combination therapy, the present invention also contemplates monotherapies using kinase inhibitors alone and monotherapies using kinase activators alone to treat cancer. Therefore, in another aspect, provided herein are methods of treating a cancer in a patient comprising administering to the patient an inhibitor of the activity of a kinase selected from the group consisting of GRK7, EGFR, RET, and BRSK1, and methods of treating a cancer in a patient comprising administering to the patient an activator of the activity of a kinase selected from the group consisting of DDR2, CDK7, MINK1, DAPK3, and MAPK3.

Inhibitors of the activity of a kinase selected from the group consisting of GRK7, EGFR, RET, and BRSK1, that can be employed in the methods described herein are described in Section 5.6, infra.

Activators of the activity of a kinase selected from the group consisting of DDR2, CDK7, MINK1, DAPK3, and MAPK3, that can be employed in the methods described herein are described in Section 5.7, infra.

Immunotherapies that promote immune response that can be employed in the methods described herein are described in Section 5.10, infra.

In some embodiments, the cancer to be treated is a blood cancer. The blood cancer can be a leukemia, a lymphoma, a myeloma, or a combination thereof. A blood cancer that can be treated in accordance with the methods described in this disclosure can be, but is not limited to: acute lymphoblastic leukemia, chronic lymphocytic leukemia, acute myelogenous leukemia, chronic myelogenous leukemia, hairy cell leukemia, T-cell prolymphocytic leukemia, Large granular lymphocytic leukemia, adult T-cell leukemia, plasma cell leukemia, Hodgkin lymphoma, Non-Hodgkin lymphoma, or multiple myeloma

In other embodiments, the cancer to be treated is a solid tumor cancer. The solid tumor cancer can be, but is not limited to, a sarcoma, a carcinoma, a lymphoma, a germ cell tumor, a blastoma, or a combination thereof. A solid tumor cancer that can be treated in accordance with the methods described in this disclosure can be, but is not limited to: breast cancer, lung cancer, ovary cancer, stomach cancer, pancreatic cancer, larynx cancer, esophageal cancer, testes cancer, liver cancer, parotid cancer, biliary tract cancer, colon cancer, rectum cancer, cervix cancer, uterus cancer, endometrium cancer, renal cancer, bladder cancer, prostate cancer, thyroid cancer, melanoma, or non-small cell lung cancer. In a specific embodiment, the cancer is lung cancer (e.g., non-small cell lung cancer), thyroid cancer, or melanoma.

In a specific embodiment, the patient's cancer is resistant to a therapy for the cancer previously administered to the patient. In some embodiments, the therapy for the cancer previously administered to the patient is chemotherapy. In other embodiments, the therapy for the cancer previously administered to the patient is radiation therapy.

In certain embodiments, the methods of treating a cancer as described above involve the killing or inhibition of proliferation of cancer cells, cancer stem cells, cancer progenitor cells, and/or cancer initiating cells, which do not have detectable MHC expression or have low levels of MHC expression (e.g., the cancer stem cells described in International Patent Application Publication No. WO 2011/038300 Al). In such embodiments, inhibition of the activity of a kinase selected from the group consisting of GRK7, EGFR, RET, and BRSK1, or activation of the activity of a kinase selected from the group consisting of DDR2, CDK7, MINK1, DAPK3, and MAPK3, upregulates class I MHC gene expression on cancer cells, cancer stem cells, cancer progenitor cells, and/or cancer initiating cells (in human patients, such inhibition upregulates HLA-A expression, and preferably also upregulates HLA-B expression and HLA-C expression on cancer cells, cancer stem cells, cancer progenitor cells, and/or cancer initiating cells).

### 5.2. Treatment of Infectious Disease

In another aspect, provided herein are methods of treating an infection in a patient comprising: (i) administering to the patient an inhibitor of the activity of a kinase selected from the group consisting of GRK7, EGFR, RET, and BRSK1, and (ii) administering to the patient an immunotherapy that promotes an immune response against the infection. According to the invention, and without intending to be bound by a mechanism, inhibition of the activity of a kinase selected from the group consisting of GRK7, EGFR, RET, and BRSK1 upregulates class I MHC gene expression on infected cells (in human patients, such inhibition upregulates HLA-A expression, and preferably also upregulates HLA-B expression and HLA-C expression on infected cells). In various embodiments, the inhibitor is administered in a subclinical amount. A subclinical amount of the inhibitor refers to an amount of the inhibitor at which no clinical effect or less than optimal clinical effect is detected when the inhibitor is administered alone (i.e., not in combination with the immunotherapy). In specific embodiments, the subclinical amount is lower than the amount of the inhibitor commonly used in the standard-of-care therapy for the infection to be treated. In specific embodiments wherein the inhibitor is FDA (Food and Drug Administration)-approved for treating the infection, the subclinical amount is lower than the FDA-approved amount for treating the infection.

In another aspect, provided herein are methods of generating a population of antigen-presenting cells for therapeutic administration to a patient having an infection, comprising culturing antigen-presenting cells that are loaded with or genetically engineered to express one or more immunogenic peptides or proteins derived from one or more antigens of the pathogen causing the infection in the presence of an inhibitor of the activity of a kinase selected from the group consisting of GRK7, EGFR, RET, and BRSK1. In another aspect, provided herein are methods of treating an infection in a patient comprising generating a population of antigen-presenting cells according to such a method and administering to the patient the population of antigen-presenting cells. The antigen-presenting cells can be, for example, dendritic cells, cytokine-activated monocytes, or PBMCs. Preferably, the antigen-presenting cells are dendritic cells. Preferably, the antigen-presenting cells are autologous to the human patient (e.g., dendritic cells autologous to the human patient).

In another ex vivo embodiment, provided herein are methods of generating a population of antigen-specific T cells for therapeutic administration to a patient having an infection, comprising co-culturing T cells with antigen-presenting cells that are loaded with or genetically engineered to express one or more immunogenic peptides or proteins derived from one or more antigens of the pathogen causing the infection in the presence of an inhibitor of the activity of a kinase selected from the group consisting of GRK7, EGFR, RET, and BRSK1. In another aspect, provided herein are methods of treating an infection in a patient comprising generating a population of antigen-specific T cells according to such a method and administering to the patient the population of antigen-specific T cells. The antigen-presenting cells can be, for example, dendritic cells, cytokine-activated monocytes, or PBMCs. Preferably, the antigen-presenting cells are dendritic cells. Preferably, the antigen-presenting cells are autologous to the human patient (e.g., dendritic cells autologous to the human patient).

In another aspect, provided herein are methods of treating an infection in a patient comprising: (i) administering to the patient an activator of the activity of a kinase selected from the group consisting of DDR2, CDK7, MINK1, DAPK3, and MAPK3, and (ii) administering to the patient an immunotherapy that promotes an immune response against the infection. According to the invention, and without intending to be bound by a mechanism, activation of the activity of a kinase selected from the group consisting of DDR2, CDK7, MINK1, DAPK3, and MAPK3 upregulates class I MHC gene expression on infected cells (in human patients, such activation upregulates HLA-A expression, and preferably also upregulates HLA-B expression and HLA-C expression on infected cells). In various embodiments, the activator is administered in a subclinical amount. A subclinical amount of the activator refers to an amount of the activator at which no clinical effect or less than optimal clinical effect is detected when the inhibitor is administered alone (i.e., not in combination with the immunotherapy). In specific embodiments, the subclinical amount is lower than the amount of the activator commonly used in the standard-of-care therapy for the infection to be treated. In specific embodiments wherein the activator is FDA -approved for treating the infection, the subclinical amount is lower than the FDA-approved amount for treating the infection.

In another aspect, provided herein are methods of generating a population of antigen-presenting cells for therapeutic administration to a patient having an infection, comprising culturing antigen-presenting cells that are loaded with or genetically engineered to express one or more immunogenic peptides or proteins derived from one or more antigens of the pathogen causing the infection in the presence of an activator of the activity of a kinase selected from the group consisting of DDR2, CDK7, MINK1, DAPK3, and MAPK3. In another aspect, provided herein are methods of treating an infection in a patient comprising generating a population of antigen-presenting cells according to such a method and administering to the patient the population of antigen-presenting cells. The antigen-presenting cells can be, for example, dendritic cells, cytokine-activated monocytes, or PBMCs. Preferably, the antigen-presenting cells are dendritic cells. Preferably, the antigen-presenting cells are autologous to the human patient (e.g., dendritic cells autologous to the human patient).

In another ex vivo embodiment, provided herein are methods of generating a population of antigen-specific T cells for therapeutic administration to a patient having an infection, comprising co-culturing T cells with antigen-presenting cells that are loaded with or genetically engineered to express one or more immunogenic peptides or proteins derived from one or more antigens of the pathogen causing the infection in the presence of an activator of the activity of a kinase selected from the group consisting of DDR2, CDK7, MINK1, DAPK3, and MAPK3. In another aspect, provided herein are methods of treating an infection in a patient comprising generating a population of antigen-specific T cells according to such a method and administering to the patient the population of antigen-specific T cells. The antigen-presenting cells can be, for example, dendritic cells, cytokine-activated monocytes, or PBMCs. Preferably, the antigen-presenting cells are dendritic cells. Preferably, the antigen-presenting cells are autologous to the human patient (e.g., dendritic cells autologous to the human patient).

While the methods of treating an infection described in this disclosure are largely methods of combination therapy, the present invention also contemplates monotherapies using kinase inhibitors alone and monotherapies using kinase activators alone to treat infection. Therefore, in another aspect, provided herein are methods of treating an infection in a patient comprising administering to the patient an inhibitor of the activity of a kinase selected from the group consisting of GRK7, EGFR, RET, and BRSK1, and methods of treating an infection in a patient comprising administering to the patient an activator of the activity of a kinase selected from the group consisting of DDR2, CDK7, MINK1, DAPK3, and MAPK3.

Inhibitors of the activity of a kinase selected from the group consisting of GRK7, EGFR, RET, and BRSK1, that can be employed in the methods described herein are described in Section 5.6, infra.

Activators of the activity of a kinase selected from the group consisting of DDR2, CDK7, MINK1, DAPK3, and MAPK3, that can be employed in the methods described herein are described in Section 5.7, infra.

Immunotherapies that promote immune response that can be employed in the methods described herein are described in Section 5.10, infra.

In certain embodiments, the infection to be treated is an infection with a virus, bacterium, fungus, helminth or protist. In specific embodiments, the infection is an infection with a virus, such as herpesvirus, cytomegalovirus, Epstein Bar virus, polyoma virus, polyoma BK virus, John Cunningham virus, adenovirus, human immunodeficiency virus, influenza virus, ebola virus, poxvirus, norovirus, rotavirus, rhabdovirus, or paramyxovirus, etc. In a specific embodiment, the infection is an infection with herpesvirus. In another specific embodiment, the infection is an infection with cytomegalovirus. In another specific embodiment, the infection is an infection with Epstein Bar virus. In another specific embodiment, the infection is an infection with polyoma virus.

In a specific embodiment, the patient's infection is resistant to a therapy for the infection previously administered to the patient. In some embodiments, the therapy for the infection previously administered to the patient is antibiotics. In other embodiments, the therapy for the infection previously administered to the patient is anti-viral therapy.

### 5.3. Treatment of Autoimmune Disease

In another aspect, provided herein are methods of treating an autoimmune disease in a patient comprising: (i) administering to the patient an activator of the activity of a kinase selected from the group consisting of GRK7, EGFR, RET, and BRSK1, and optionally (ii) administering to the patient an immunosuppressive therapy that suppresses the immune response associated with the autoimmune disease. According to the invention, and without intending to be bound by a mechanism, activation of the activity of a kinase selected from the group consisting of GRK7, EGFR, RET, and BRSK1 downregulates class I MHC gene expression on cells to which an autoimmune response is directed (in human patients, such activation downregulates HLA-A expression, and preferably also downregulates HLA-B expression and HLA-C expression on cells to which an autoimmune response is directed). In various embodiments, the activator is administered in a subclinical amount. A subclinical amount of the activator refers to an amount of the activator at which no clinical effect or less than optimal clinical effect is detected when the activator is administered alone (i.e., not in combination with the immunosuppressive therapy). In specific embodiments, the subclinical amount is lower than the amount of the activator commonly used in the standard-of-care therapy for the autoimmune disease to be treated. In specific embodiments wherein the activator is FDA (Food and Drug Administration)-approved for treating the autoimmune disease, the subclinical amount is lower than the FDA-approved amount for treating the autoimmune disease.

In another aspect, provided herein are methods of treating an autoimmune disease in a patient comprising: (i) administering to the patient an inhibitor of the activity of a kinase selected from the group consisting of DDR2, CDK7, MINK1, DAPK3, and MAPK3, and optionally (ii) administering to the patient an immunosuppressive therapy that suppresses the immune response associated with the autoimmune disease. According to the invention, and without intending to be bound by a mechanism, inhibition of the activity of a kinase selected from the group consisting of DDR2, CDK7, MINK1, DAPK3, and MAPK3 downregulates class I MHC gene expression on cells to which an autoimmune response is directed (in human patients, such inhibition downregulates HLA-A expression, and preferably also downregulates HLA-B expression and HLA-C expression on cells to which an autoimmune response is directed). In various embodiments, the inhibitor is administered in a subclinical amount. A subclinical amount of the inhibitor refers to an amount of the inhibitor at which no clinical effect or less than optimal clinical effect is detected when the inhibitor is administered alone (i.e., not in combination with the immunosuppressive therapy). In specific embodiments, the subclinical amount is lower than the amount of the inhibitor commonly used in the standard-of-care therapy for the autoimmune disease to be treated. In specific embodiments wherein the inhibitor is FDA-approved for treating the autoimmune disease, the subclinical amount is lower than the FDA-approved amount for treating the autoimmune disease.

While the methods of treating an autoimmune disease described in this disclosure are largely methods of combination therapy, the present invention also contemplates monotherapies using kinase inhibitors alone and monotherapies using kinase activators alone to treat autoimmune disease. Therefore, in another aspect, provided herein are methods of treating an autoimmune disease in a patient comprising administering to the patient an activator of the activity of a kinase selected from the group consisting of GRK7, EGFR, RET, and BRSK1, and methods of treating an autoimmune disease in a patient comprising administering to the patient an inhibitor of the activity of a kinase selected from the group consisting of DDR2, CDK7, MINK1, DAPK3, and MAPK3.

Activators of the activity of a kinase selected from the group consisting of GRK7, EGFR, RET, and BRSK1, that can be employed in the methods described herein are described in Section 5.8, infra.

Inhibitors of the activity of a kinase selected from the group consisting of DDR2, CDK7, MINK1, DAPK3, and MAPK3, that can be employed in the methods described herein are described in Section 5.9, infra.

Immunosuppressive therapies that suppress immune response that can be employed in the methods described herein are described in Section 5.11, infra.

An autoimmune disease that can be treated in accordance with the methods described in this disclosure can be, but is not limited to: Addison's disease, alopecia areata, ankylosing spondylitis, celiac sprue disease, Graves' disease, Hashimoto's thyroiditis, inflammatory bowel disease, lupus, multiple sclerosis, polymyalgia rheumatic, psoriasis, reactive arthritis, rheumatoid arthritis, scleroderma, Sjogren's syndrome, systemic lupus erythematosus, type 1 diabetes, temporal arteritis, vasculitis, or vitiligo. In a specific embodiment, the autoimmune disease is multiple sclerosis, type 1 diabetes, ankylosing spondylitis, or Hashimoto's thyroiditis.

In a specific embodiment, the patient's autoimmune disease is resistant to a therapy for the autoimmune disease previously administered to the patient. In some embodiments, the therapy for the autoimmune disease previously administered to the patient is an immunosuppressive therapy, such as those immunosuppressive therapies described in Section 5.11, supra.

### 5.4. Treatment of Graft-versus-Host Diseases

In another aspect, provided herein are methods of treating graft-versus-host disease (GvHD) in a patient comprising: (i) administering to the patient an activator of the activity of a kinase selected from the group consisting of GRK7, EGFR, RET, and BRSK1, and optionally (ii) administering to the patient an immunosuppressive therapy that suppresses the immune response associated with the GvHD. According to the invention, and without intending to be bound by a mechanism, activation of the activity of a kinase selected from the group consisting of GRK7, EGFR, RET, and BRSK1 downregulates class I MHC gene expression on grafted cells (in human patients, such activation downregulates HLA-A expression, and preferably also downregulates HLA-B expression and HLA-C expression on grafted cells). In various embodiments, the activator is administered in a subclinical amount. A subclinical amount of the activator refers to an amount of the activator at which no clinical effect or less than optimal clinical effect is detected when the activator is administered alone (i.e., not in combination with the immunosuppressive therapy). In specific embodiments, the subclinical amount is lower than the amount of the activator commonly used in the standard-of-care therapy for the GvHD to be treated. In specific embodiments wherein the activator is FDA (Food and Drug Administration)-approved for treating the GvHD, the subclinical amount is lower than the FDA-approved amount for treating the GvHD.

In another aspect, provided herein are methods of treating a GvHD in a patient comprising: (i) administering to the patient an inhibitor of the activity of a kinase selected from the group consisting of DDR2, CDK7, MINK1, DAPK3, and MAPK3, and optionally (ii) administering to the patient an immunosuppressive therapy that suppresses the immune response associated with the GvHD. According to the invention, and without intending to be bound by a mechanism, inhibition of the activity of a kinase selected from the group consisting of DDR2, CDK7, MINK1, DAPK3, and MAPK3 downregulates class I MHC gene expression on grafted cells (in human patients, such inhibition downregulates HLA-A expression, and preferably also downregulates HLA-B expression and HLA-C expression on grafted cells). In various embodiments, the inhibitor is administered in a subclinical amount. A subclinical amount of the inhibitor refers to an amount of the inhibitor at which no clinical effect or less than optimal clinical effect is detected when the inhibitor is administered alone (i.e., not in combination with the immunosuppressive therapy). In specific embodiments, the subclinical amount is lower than the amount of the inhibitor commonly used in the standard-of-care therapy for the GvHD to be treated. In specific embodiments wherein the inhibitor is FDA -approved for treating the GvHD, the subclinical amount is lower than the FDA-approved amount for treating the GvHD.

While the methods of treating a GvHD described in this disclosure are largely methods of combination therapy, the present invention also contemplates monotherapies using kinase inhibitors alone and monotherapies using kinase activators alone to treat GvHD. Therefore, in another aspect, provided herein are methods of treating a GvHD in a patient comprising administering to the patient an activator of the activity of a kinase selected from the group consisting of GRK7, EGFR, RET, and BRSK1, and methods of treating a GvHD in a patient comprising administering to the patient an inhibitor of the activity of a kinase selected from the group consisting of DDR2, CDK7, MINK1, DAPK3, and MAPK3.

Activators of the activity of a kinase selected from the group consisting of GRK7, EGFR, RET, and BRSK1, that can be employed in the methods described herein are described in Section 5.8, infra.

Inhibitors of the activity of a kinase selected from the group consisting of DDR2, CDK7, MINK1, DAPK3, and MAPK3, that can be employed in the methods described herein are described in Section 5.9, infra.

Immunosuppressive therapies that suppress immune response that can be employed in the methods described herein are described in Section 5.11, infra.

In some embodiments, the GvHD to be treated is an acute GvHD. In other embodiments, the GvHD to be treated is a chronic GvHD.

In a specific embodiment, the GvHD to be treated results from an allogeneic donor leukocyte infusion. In another specific embodiment, the GvHD to be treated results from an allogeneic hematopoietic stem cell transplantation (e.g., a bone marrow transplantation, a peripheral blood stem cell transplantation, or a cord blood transplantation). In another specific embodiment, the GvHD to be treated results from an allogeneic blood transfusion.

In a specific embodiment, the patient's GvHD is resistant to a therapy for the GvHD previously administered to the patient. In some embodiments, the therapy for the GvHD previously administered to the patient is an immunosuppressive therapy, such as those immunosuppressive therapies described in Section 5.11, supra.

### 5.5. Reduction of Risk of or Prevention of Solid Organ Transplant Rejection

In another aspect, provided herein are methods of reducing the risk of (e.g., prevention of) solid organ transplant rejection in a patient comprising: (i) administering to the patient an activator of the activity of a kinase selected from the group consisting of GRK7, EGFR, RET, and BRSK1, and optionally (ii) administering to the patient an immunosuppressive therapy that suppresses the immune response against the solid organ transplant. According to the invention, and without intending to be bound by a mechanism, activation of the activity of a kinase selected from the group consisting of GRK7, EGFR, RET, and BRSK1 downregulates class I MHC gene expression on solid organ transplant cells (in human patients, such activation downregulates HLA-A expression, and preferably also downregulates HLA-B expression and HLA-C expression on solid organ transplant cells). In various embodiments, the activator is administered in a subclinical amount. A subclinical amount of the activator refers to an amount of the activator at which no clinical effect or less than optimal clinical effect is detected when the activator is administered alone (i.e., not in combination with the immunosuppressive therapy). In specific embodiments, the subclinical amount is lower than the amount of the activator commonly used in the standard-of-care therapy for reducing the risk of (e.g., prevention of) solid organ transplant rejection. In specific embodiments wherein the activator is FDA (Food and Drug Administration)-approved for reducing the risk of (e.g., prevention of) solid organ transplant rejection , the subclinical amount is lower than the FDA-approved amount for reducing the risk of (e.g., prevention of) solid organ transplant rejection.

In another aspect, provided herein are methods of reducing the risk of (e.g., prevention of) solid organ transplant rejection in a patient comprising: (i) administering to the patient an inhibitor of the activity of a kinase selected from the group consisting of DDR2, CDK7, MINK1, DAPK3, and MAPK3, and optionally (ii) administering to the patient an immunosuppressive therapy that suppresses the immune response against the solid organ transplant. According to the invention, and without intending to be bound by a mechanism, inhibition of the activity of a kinase selected from the group consisting of DDR2, CDK7, MINK1, DAPK3, and MAPK3 downregulates class I MHC gene expression on solid organ transplant cells (in human patients, such inhibition downregulates HLA-A expression, and preferably also downregulates HLA-B expression and HLA-C expression on solid organ transplant cells). In various embodiments, the inhibitor is administered in a subclinical amount. A subclinical amount of the inhibitor refers to an amount of the inhibitor at which no clinical effect or less than optimal clinical effect is detected when the inhibitor is administered alone (i.e., not in combination with the immunosuppressive therapy). In specific embodiments, the subclinical amount is lower than the amount of the inhibitor commonly used in the standard-of-care therapy for reducing the risk of (e.g., prevention of) solid organ transplant rejection. In specific embodiments wherein the inhibitor is FDA -approved for reducing the risk of (e.g., prevention of) solid organ transplant rejection, the subclinical amount is lower than the FDA-approved amount for reducing the risk of (e.g., prevention of) solid organ transplant rejection.

While the methods of reducing the risk of (e.g., prevention of) solid organ transplant rejection described in this disclosure are largely methods of combination therapy, the present invention also contemplates monotherapies using kinase inhibitors alone and monotherapies using kinase activators alone for reducing the risk of (e.g., prevention of) solid organ transplant rejection. Therefore, in another aspect, provided herein are methods of reducing the risk of (e.g., prevention of) solid organ transplant rejection in a patient comprising administering to the patient an activator of the activity of a kinase selected from the group consisting of GRK7, EGFR, RET, and BRSK1, and methods of reducing the risk of (e.g., prevention of) solid organ transplant rejection in a patient comprising administering to the patient an inhibitor of the activity of a kinase selected from the group consisting of DDR2, CDK7, MINK1, DAPK3, and MAPK3.

Activators of the activity of a kinase selected from the group consisting of GRK7, EGFR, RET, and BRSK1, that can be employed in the methods described herein are described in Section 5.8, infra.

Inhibitors of the activity of a kinase selected from the group consisting of DDR2, CDK7, MINK1, DAPK3, and MAPK3, that can be employed in the methods described herein are described in Section 5.9, infra.

Immunosuppressive therapies that suppress immune response that can be employed in the methods described herein are described in Section 5.11, infra.

In specific embodiments, the solid organ transplant is a kidney transplant, a liver transplant, a heart transplant, an intestinal transplant, a pancreas transplant, a lung transplant, a small bowel transplant, a thymus transplant, or a combination thereof

In a specific embodiment, the patient's solid organ transplant is resistant to a therapy for reducing the risk of (e.g., prevention of) solid organ transplant rejection previously administered to the patient. In some embodiments, the therapy for reducing the risk of (e.g., prevention of) solid organ transplant rejection previously administered to the patient is an immunosuppressive therapy, such as those immunosuppressive therapies described in Section 5.11, supra.

### 5.6. Inhibitors of Kinases that Negatively Regulate MHC Class I Expression

In some embodiments, the inhibitor of the activity of a kinase selected from the group consisting of GRK7, EGFR, RET, and BRSK1, is a small molecule inhibitor. In other embodiments, the inhibitor is an antibody or an antigen-binding fragment thereof that specifically binds to the kinase. In specific embodiments, the antibody or antigen-binding fragment thereof antagonizes the activity of the kinase. Antibodies or an antigen-binding fragments thereof that can be the inhibitor include, but are not limited to, monoclonal antibodies, polyclonal antibodies, multispecific antibodies (e.g., bispecific antibodies), and antibody fragments retaining antigen-binding activity, such as Fv, Fab, Fab′, F(ab′)₂, diabodies, linear antibodies, single-chain antibody molecules (e.g., single chain fragment variable fragment (scFv)), multispecific antibodies formed from antibody fragments. In a specific embodiment, the antibody is a monoclonal antibody, for example, a neutralizing monoclonal antibody. In other embodiments, the inhibitor is an oligonucleotide such as an aptamer, an shRNA, miRNA, siRNA, or antisense DNA.

In a specific embodiment, the kinase is EGFR and the inhibitor is erlotinib, gefitinib, afatanib, or lapatinib.

In another specific embodiment, the kinase is RET and the inhibitor is regorafenib, danusertib, cabozantinib, or AST487 (1-[4-[(4-ethylpiperazin-1-yl)methyl]-3-(trifluoromethyl)phenyl]-3-[4-[6-(methylamino)pyrimidin-4-yl]oxyphenyl]urea).

### 5.7. Activators of Kinases that Positively Regulate MHC Class I Expression

In some embodiments, the activator of the activity of a kinase selected from the group consisting of DDR2, CDK7, MINK1, DAPK3, and MAPK3, is a soluble ligand (e.g., an activating protein ligand) of the kinase (e.g., where the kinase is a receptor), or a soluble ligand (e.g., an activating protein ligand) of a receptor that activates the kinase in vivo. In other embodiments, the activator is an antibody or an antigen-binding fragment thereof that specifically binds to the kinase. In specific embodiments, the antibody or antigen-binding fragment thereof agonizes the activity of the kinase. Antibodies or an antigen-binding fragments thereof that can be the activator include, but are not limited to, monoclonal antibodies, polyclonal antibodies, multispecific antibodies (e.g., bispecific antibodies), and antibody fragments retaining antigen-binding activity, such as Fv, Fab, Fab′, F(ab′)₂, diabodies, linear antibodies, single-chain antibody molecules (e.g., scFv), multispecific antibodies formed from antibody fragments. In a specific embodiment, the antibody is a monoclonal antibody.

### 5.8. Activators of Kinases that Negatively Regulate MHC Class I Expression

In some embodiments, the activator of the activity of a kinase selected from the group consisting of GRK7, EGFR, RET, and BRSK1, is a soluble ligand (e.g., an activating protein ligand) of the kinase (e.g., where the kinase is a receptor), or a soluble ligand (e.g., an activating protein ligand) of a receptor that activates the kinase in vivo. In other embodiments, the activator is an antibody or an antigen-binding fragment thereof that specifically binds to the kinase. In specific embodiments, the antibody or antigen-binding fragment thereof agonizes the activity of the kinase. Antibodies or an antigen-binding fragments thereof that can be the activator include, but are not limited to, monoclonal antibodies, polyclonal antibodies, multispecific antibodies (e.g., bispecific antibodies), and antibody fragments retaining antigen-binding activity, such as Fv, Fab, Fab′, F(ab′)₂, diabodies, linear antibodies, single-chain antibody molecules (e.g., scFv), multispecific antibodies formed from antibody fragments. In a specific embodiment, the antibody is a monoclonal antibody. In a specific embodiment, the kinase is EGFR and the activator is EGF protein that turns on EGFR.

### 5.9. Inhibitors of Kinases that Positively Regulate MHC Class I Expression

In some embodiments, the inhibitor of the activity of a kinase selected from the group consisting of DDR2, CDK7, MINK1, DAPK3, and MAPK3, is a small molecule inhibitor. In other embodiments, the inhibitor is an antibody or an antigen-binding fragment thereof that specifically binds to the kinase. In specific embodiments, the antibody or antigen-binding fragment thereof antagonizes the activity of the kinase. Antibodies or an antigen-binding fragments thereof that can be the inhibitor include, but are not limited to, monoclonal antibodies, polyclonal antibodies, multispecific antibodies (e.g., bispecific antibodies), and antibody fragments retaining antigen-binding activity, such as Fv, Fab, Fab′, F(ab′)₂, diabodies, linear antibodies, single-chain antibody molecules (e.g., single chain fragment variable fragment (scFv)), multispecific antibodies formed from antibody fragments. In a specific embodiment, the antibody is a monoclonal antibody, for example, a neutralizing monoclonal antibody. In other embodiments, the inhibitor is an oligonucleotide such as an aptamer, an shRNA, miRNA, siRNA, or antisense DNA.

In a specific embodiment, the kinase is DDR2 and the inhibitor is dasatinib.

In another specific embodiment, the kinase is CDK7 and the inhibitor is BS-181 HCl (N5-(6-aminohexyl)-N7-benzyl-3-isopropylpyrazolo[1,5-a]pyrimidine-5,7-diamine hydrochloride).

In another specific embodiment, the kinase is DAPK3 and the inhibitor is 324788 ((4Z)-4-(3-Pyridylmethylene)-2-styryl-oxazol-5-one).

In another specific embodiment, the kinase is MAPK3 and the inhibitor is ulixertinib.

### 5.10. Immunotherapies that Promote Immune Response

An immunotherapy promotes an immune response if it initiates an immune response or enhances a pre-existing immune response. In some embodiments of the methods of treating a cancer and the methods of treating an infection, which comprise administering to the patient an immunotherapy that promotes an immune response, the immunotherapy initiates an immune response against the cancer or the infection (as the case may be). In other embodiments of the methods of treating a cancer and the methods of treating an infection, which comprise administering to the patient an immunotherapy that promotes an immune response, the immunotherapy enhances a pre-existing immune response against the cancer or the infection (as the case may be)

In various embodiments, the immunotherapy can be a vaccine, an immune checkpoint blockade, an adoptive immunotherapy, a TCR (T-Cell Receptor) mimic antibody, a TCR based construct, an interferon (preferably interferon alpha or gamma), an anti-CD47 antibody, a SIRP alpha antagonist, an HDAC inhibitor, a cytokine, a TLR (Toll-Like Receptor) agonist, an epigenetic modulator that upregulates the expression of one or more MHCs (Major Histocompatibility Complexes) or upregulates antigen presentation, or a combination thereof.

In some embodiments, the immunotherapy is a vaccine. The vaccine can be any biological preparation that stimulates or elicits an endogenous immune response in the human patient against one or more antigens of the cancer or the pathogen causing the infection (as the case may be), such as, but are not limited to the ones described in Melief et al., 2015, J Clin Invest 125:3401-3412; Melero et al., 2014, Nat Rev Clin Oncol 11:509-524; and Guo et al., 2013, Adv Cancer Res 119:421-475; Nabel, 2013, N Engl J Med 368:551-560; and Saroja et al., 2011, Int J Pharm Investig 1: 64-74. In a specific embodiment, the vaccine comprises a peptide(s) or a protein(s) derived from the one or more antigens of the cancer or the pathogen causing the infection (as the case may be). In another specific embodiment, the vaccine comprises a nucleotide (e.g., a vector) expressing a peptide or a protein derived from the one or more antigens of the cancer or the pathogen causing the infection (as the case may be). In another specific embodiment, the vaccine is an antigen-presenting cell vaccine. In certain embodiments, the antigen-presenting cells in the antigen-presenting cell vaccine are loaded with one or more immunogenic peptides or proteins derived from one or more antigens of the cancer or the pathogen causing the infection (as the case may be). In other embodiments, the antigen-presenting cells in the antigen-presenting cell vaccine are genetically engineered to express one or more immunogenic peptides or proteins derived from one or more antigens of the cancer or the pathogen causing the infection (as the case may be). In preferred embodiments, the antigen-presenting cell vaccine is a dendritic cell vaccine.

In other embodiments, the immunotherapy is an immune checkpoint blockade. In specific embodiments, the immune checkpoint blockade is an antibody or an antigen-binding fragment thereof that specifically binds to and reduces the activity of an immune checkpoint protein. In a specific embodiment, the immune checkpoint blockade is an antibody or an antigen-binding fragment thereof that specifically binds to and blocks the activity of an immune checkpoint protein. Antibodies or an antigen-binding fragments thereof that can be the immune checkpoint blockade include, but are not limited to, monoclonal antibodies, polyclonal antibodies, multispecific antibodies (e.g., bispecific antibodies), and antibody fragments retaining antigen-binding activity, such as Fv, Fab, Fab′, F(ab′)₂, diabodies, linear antibodies, single-chain antibody molecules (e.g., scFv), multispecific antibodies formed from antibody fragments. In a specific embodiment, the antibody is a monoclonal antibody. In certain embodiments, the immune checkpoint blockade inhibits the activity of CTLA-4, PD-1, PD-L1, PD-L2, TIM-3, or LAG-3. In specific embodiments, the immune checkpoint blockade is an antibody or antigen-binding fragment thereof that specifically binds to and reduces the activity of CTLA-4, PD-1, PD-L1, PD-L2, TIM-3, or LAG-3. In a specific embodiment, the immune checkpoint blockade is tremelimumab. In another specific embodiment, the immune checkpoint blockade is nivolumab. In another specific embodiment, the immune checkpoint blockade is pembrolizumab. In another specific embodiment, the immune checkpoint blockade is ipilimumab.

In other embodiments, the immunotherapy is an adoptive immunotherapy, such as an adoptive T cell therapy. In specific embodiments, the adoptive T cell therapy involves the ex vivo stimulation, enrichment and/or expansion of non-genetically engineered antigen-specific T cells for infusion, for example as described in Yee, 2014, Immunol Rev 257:250-263; O'Reilly et al., 2011, Best Practice & Research Clinical Haematology 24:381-391; or O'Reilly et al., 2010, Semin Immunol 2010, 22:162-172. In other specific embodiments, the adoptive T cell therapy involves the infusion of genetically engineered T cells. In a specific embodiment, the adoptive T cell therapy is TCR-engineered T cells. A TCR-engineered T cell is a T cell that is genetically engineered to express on its surface a TCR that recognizes an antigen (which may be an intracellular antigen) of the cancer or the pathogen causing the infection (as the case may be). Preferably, a TCR expressed on the surface of a TCR-engineered T cell has high affinity for an antigen (which may be an intracellular antigen) of the cancer or the pathogen causing the infection (as the case may be). TCR-engineered T cells that can be employed in accordance with the present invention and technologies for generating TCR-engineered T cells are described in, for example, Stauss et al., 2015, Curr Opin Pharmacol 24:113-118; Sharpe and Mount, 2015, Dis Model Mech 8:337-350; Kunert et al., 2013, Front Immunol 4: 363; Stone et al., 2012, Methods Enzymol 503:189-222; and Park et al., 2011, Trends Biotechnol 29:550-557. In another specific embodiment, the adoptive T cell therapy is CAR T cells, wherein the antigen-binding domain of the CAR specifically binds to an antigen of the cancer. CARs are engineered receptors that provide both antigen binding and immune cell activation functions (Sadelain et al., 2013, Cancer Discovery 3:388-398). They usually comprise an antigen-binding domain (e.g., derived from a monoclonal antibody or the extracellular domain of a receptor), a transmembrane domain, an intracellular domain, and optionally a co-stimulatory domain. CARs can be used to graft the specificity of an antigen-binding domain onto an immune cell such as a T cell. CAR T cells are T cells that are genetically engineered to express CARs on their surface. CAR T cells that can be employed in accordance with the present invention and technologies for generating CAR T cells are described in, for example, Stauss et al., 2015, Curr Opin Pharmacol 24:113-118; Sharpe and Mount, 2015, Dis Model Mech 8:337-350; and Park et al., 2011, Trends Biotechnol 29:550-557.

In other embodiments, the immunotherapy is a TCR mimic antibody. TCR mimic antibodies are monoclonal antibodies that target against the WIC/antigen-peptide complexes presented on diseased cells (e.g., cancer cells or infected cells) (Dao et al., 2013, Oncolmmunology 2:e24678). They combine the recognition of antigen peptides (which may be peptides derived from intracellular antigens), analogous to that of a TCR, with the therapeutic potency and versatility of monoclonal antibodies. TCR mimic antibodies that can be employed in accordance with the present invention and technologies for generating TCR mimic antibodies, are described in, for example, Dubrovsky et al., 2015, Oncoimmunology 5:e1049803; Dao et al., 2013, Oncolmmunology 2:e24678; Cohen and Reiter, 2013, Antibodies, 2:517-534; and Dahan and Reiter, 2012, Expert Rev Mol Med 14:e6.

In other embodiments, the immunotherapy is a TCR based construct that encodes a soluble protein comprising the antigen recognition domain of a TCR. In certain embodiments, the immunotherapy is a soluble protein comprising the antigen recognition domain of a TCR. In preferred embodiments, the protein comprising the antigen recognition domain of a TCR comprises a second moiety for killing or inhibiting the proliferation of the cancer cells or infected cells (as the case may be) that are recognized by the TCR moiety. In a specific embodiment, the protein comprising the antigen recognition domain of a TCR is conjugated to a cytotoxic moiety. Such a cytotoxic moiety can be a cytotoxin, such as a radioisotope (e.g., a beta or alpha emitter), a cytotoxic drug (e.g., aureostatin), or a protein toxin (e.g., ricin). In another specific embodiment, the protein comprising the antigen recognition domain of a TCR also comprises an inflammatory cytokine, such as IL-2, TNF, or interferon gamma. In another specific embodiment, the protein comprising the antigen recognition domain of a TCR also comprises an antibody that specifically binds to a surface antigen on immune cells, such as T cells (e.g., an anti-CD3 antibody, such as an anti-CD3 scFv). In a further specific embodiment, the protein comprising the antigen recognition domain of a TCR is an immune mobilizing monoclonal TCR against cancer (ImmTAC). Soluble protein comprising the antigen recognition domain of a TCR and TCR based constructs that express such proteins, which can be employed in accordance with the present invention, and technologies for generating such soluble proteins and TCR based constructs are described in, for example, Oates et al., 2015, Mol Immunol 67:67-74; and Walseng et al., 2015, PLoS One 10:e0119559. The TCR based construct or the soluble protein comprising the antigen recognition domain of a TCR can be incorporated genetically or biochemically into a cell that affects the killing of the cancer, such as a T cell, a Natural Killer cell, or a monocyte.

In other embodiments, the immunotherapy is an interferon (preferably interferon alpha or gamma), an anti-CD47 antibody, a SIRP alpha antagonist, an HDAC inhibitor, a cytokine, a TLR agonist, or an epigenetic modulator that upregulates the expression of one or more MHCs (Major Histocompatibility Complexes) or upregulates antigen presentation. In a specific embodiment, the immunotherapy is an epigenetic modulator that upregulates the expression of one or more MHCs or upregulates antigen presentation that is a hypomethylating agent (e.g., azacytidine or decitabine). In another specific embodiment, the immunotherapy is an interferon that is interferon alpha or interferon gamma. In another specific embodiment, the immunotherapy is a cytokine that is IL2 (Interleukin-2), TNF (Tumor Necrosis Factor), interferon alpha or interferon gamma. In another specific embodiment, the immunotherapy is a TLR agonist that is a dsDNA (double-stranded DNA) TLR agonist. In another specific embodiment, the immunotherapy is a TLR agonist that is a dsRNA (double-stranded RNA) TLR agonist (e.g., polyinosinic-polycytidylic acid (poly(I:C)).

### 5.11. Immunosuppressive Therapies

An immunosuppressive therapy suppresses an immune response if it reduces or blocks an immune response. In some embodiments of the methods of treating an autoimmune disease, the methods of treating a GvHD, and the methods of reducing the risk of (e.g., prevention of) solid organ transplant rejection, which comprise administering to the patient an immunosuppressive therapy that suppresses an immune response, the immunosuppressive therapy reduces an immune response associated with the autoimmune disease or the GvHD or against the solid organ transplant (as the case may be). In other embodiments of the methods of treating an autoimmune disease, the methods of treating a GvHD, and the methods of reducing the risk of (e.g., prevention of) solid organ transplant rejection, which comprise administering to the patient an immunosuppressive therapy that suppresses an immune response, the immunosuppressive therapy blocks an immune response associated with the autoimmune disease or the GvHD or against the solid organ transplant (as the case may be).

The immunosuppressive therapy that can be employed in the methods of treating an autoimmune disease or a GVHD and the methods of reducing the risk of (e.g., prevention of) solid organ transplant rejection as described in this disclosure can be, but is not limited to, a glucocorticoid, a cytostatic (e.g., an alkylating agent, such as coclophosphamide, nitrosoureas, or platinum compound; or an antimetabolite, such as folic acid, purine analogue, pyrimidine analogue, protein synthesis inhibitor, methotrexate, azathioprine, mercaptopurine, fluorouracil, or a cytotoxic antibiotic) , an antibody that can antagonize the activity of immune cells or cytokines (e.g., anti-CD20 antibody, anti-CD3 antibody, anti-IL2R antibody), a drug acting on immunophilins (e.g., ciclosporin, tacrolimus, or sirolimus), interferon beta, an opiod, a TNF antagonist (e.g., etanercept, infliximab, or adalimumab), mycophenolic acid, mycophenolate, fingolimod, or myriocin.

In various embodiments, the immunosuppressive therapy can be sirolimus, everolimus, rapamycin, one or more steroids, cyclosporine, cyclophosphamide, azathioprine, mercaptopurine, fluorouracil, fludarabine, interferon beta, a TNF decoy receptor, a TNF antibody, methotrexate, a T-cell antibody, an anti-CD20 antibody, a complement inhibitor, an anti-IL6 (Interleukin-6) antibody, an anti-IL2R (Interleukin-2 Receptor) antibody, anti-thymocyte globulin, fingolimod, mycophenolate, or a combination thereof.

In some embodiments, the immunosuppressive therapy is a TNF decoy receptor (e.g., etanercept). In other embodiments, the immunosuppressive therapy is a TNF antibody (e.g., infliximab). In other embodiments, the immunosuppressive therapy is a T-cell antibody (e.g., an anti-CD3 antibody, such as OKT3). In other embodiments, the immunosuppressive therapy is an anti-CD20 antibody (e.g., rituximab). In other embodiments, the immunosuppressive therapy is a complement inhibitor (e.g., eculizumab). In other embodiments, the immunosuppressive therapy is an anti-IL2R antibody (e.g., daclizumab).

### 5.12. Routes of Administration and Dosage

The inhibitors of kinases and activators of kinases as described above may be administered to patients by a variety of routes. These include, but are not limited to, parenteral, intranasal, intratracheal, oral, intradermal, topical, intramuscular, intraperitoneal, transdermal, intravenous, intratumoral, conjunctival, subcutaneous, and pulmonary routes.

The amount of an inhibitor of kinase or an activator of a kinase described herein or a pharmaceutical composition thereof to be administered to the patient will depend on the nature of the disease and the condition of the patient, and can be determined by standard clinical techniques and the knowledge of the physician.

The precise dose and regime to be employed in a composition will also depend on the route of administration, and the seriousness of the disease, and should be decided according to the judgment of the physician and each patient's circumstance.

In embodiments of combination therapies, the inhibitor of a kinase or the activator of a kinase (as the case may be) is administered concurrently or sequentially with the administration of the immunotherapy that promotes an immune response or the immunosuppressive therapy that suppresses an immune response (as the case may be), for example, at about the same time, the same day, or same week, or same period (treatment cycle) during which the immunotherapy that promotes an immune response or the immunosuppressive therapy that suppresses an immune response is administered, or on similar dosing schedules, or on different but overlapping dosing schedules. Preferably, the inhibitor of a kinase or the activator of a kinase (as the case may be) is administered concurrently with or shortly before (e.g., about 1, 2, 3, 4, 6, 8, 10, 12, 16, 20, or 24 hours before, or about 1, 2, 3, 4, 5, 6, or 7 days before) the administration of the immunotherapy that promotes an immune response or the immunosuppressive therapy that suppresses an immune response (as the case may be), as described above. The inhibitor of a kinase or the activator of a kinase (as the case may be), and the immunotherapy that promotes an immune response or the immunosuppressive therapy that suppresses an immune response (as the case may be) can be in the same pharmaceutical formulation or in separate formulations.

In a specific embodiment of the methods of treating cancer, the inhibitor of a kinase or the activator of a kinase described in Sections 5.6 and 5.7, supra, is coupled with (e.g., conjugated to) an antibody that specifically binds to a cell surface marker uniquely expressed or expressed at higer levels (relative to non-cancerous cells) on the cancer cells, so that the inhibitor of a kinase or the activator of a kinase is delivered specifically to the cancer cells. In another specific embodiment of the methods of treating cancer, the inhibitor of a kinase or the activator of a kinase described in Sections 5.6 and 5.7, supra, is coupled with (e.g., conjugated to) an antibody that specifically binds to a cell surface marker uniquely expressed or expressed at higher levels (relative to cells that are not cancer stem cells, cancer progenitor cells, and/or cancer initiating cells) on cancer stem cells, cancer progenitor cells, and/or cancer initiating cells of the cancer, so that the inhibitor of a kinase or the activator of a kinase is delivered specifically to the cancer stem cells, cancer progenitor cells, and/or cancer initiating cells of the cancer.

In a specific embodiment of the methods of treating an infection, the inhibitor of a kinase or the activator of a kinase described in Sections 5.6 and 5.7, supra, is coupled with (e.g., conjugated to) an antibody that specifically binds to a cell surface marker uniquely expressed or expressed at higher levels (relative to uninfected cells) on the infected cells, so that the inhibitor of a kinase or the activator of a kinase is delivered specifically to the infected cells.

In a specific embodiment of the methods of treating an autoimmune disease, the inhibitor of a kinase or the activator of a kinase described in Sections 5.8 and 5.9, supra, is coupled with (e.g., conjugated to) an antibody that specifically binds to a cell surface marker uniquely expressed or expressed at higher levels (relative to wild-type cells) on cells to which an autoimmune response is derected, so that the inhibitor of a kinase or the activator of a kinase is delivered specifically to the cells that are the target of an autoimmune response.

In a specific embodiment of the methods of treating GvHD, the inhibitor of a kinase or the activator of a kinase described in Sections 5.8 and 5.9, supra, is coupled with (e.g., conjugated to) an antibody that specifically binds to a cell surface marker uniquely expressed or expressed at higher levels (relative to non-grafted cells) on grafted cells, so that the inhibitor of a kinase or the activator of a kinase is delivered specifically to the grafted cells.

In a specific embodiment of the methods of reducing the risk of solid organ transplant rejection, the inhibitor of a kinase or the activator of a kinase described in Sections 5.8 and 5.9, supra, is coupled with (e.g., conjugated to) an antibody that specifically binds to a cell surface marker uniquely expressed or expressed at higher levels (relative to cells not of the transplant) on the solid organ transplant, so that the inhibitor of a kinase or the activator of a kinase is delivered specifically to the solid organ transplant.

### 5.13. Patients

The patient referred to in this disclosure, can be, but is not limited to, a human or non-human vertebrate such as a wild, domestic or farm animal. In certain embodiments, the patient is a mammal, e.g., a human, a cow, a dog, a cat, a goat, a horse, a sheep, a pig, a rabbit, a rat, or a mouse. In a preferred embodiment, the patient is a human patient.

In a specific embodiment, the human patient is an adult (at least age 16). In another specific embodiment, the human patient is an adolescent (age 12-15). In another specific embodiment, the patient is a child (under age 12).

### 6. EXAMPLES

The following non-limiting examples report the discovery of a set of kinases that are negative regulators of class I MHC gene expression, and a different set of kinases that are positive regulators of class I MHC gene expression. In addition, the examples demonstrate that combination treatments using an inhibitor of a kinase that negatively regulates class I MHC gene expression and an immunotherapy have synergistic effect in killing cancer cells.

References to antibody BB7 in this application are references to antibody BB7.2.

### 6.1. Example 1: The Regulation of the Expression of Human Major Histocompatibility Class I Molecules on Cancer Cells by Kinases

6.1.1. Introduction

The major histocompatibility complex I (MHC-I) presents antigenic peptides to tumor specific CD8+T cells. The regulation of MHC-I by kinases is largely unstudied, even though many patients with cancer are receiving therapeutic kinase inhibitors. Regulators of cell surface human leukocyte antigen (HLA) levels were discovered using a pooled human kinome short hairpin RNA (shRNA) interference-based approach. Highly scoring hits were subsequently validated by additional RNA interference (RNAi) and pharmacologic inhibitors. Mitogen-activated protein kinase kinase 1 (MAP2K1; also referred to as MEK), epidermal growth factor receptor (EGFR), and ret proto-oncogene (RET) were validated as negative regulators of MHC-I expression and antigen presentation machinery in multiple cancer types acting through an extracellular regulated MAP kinase (ERK) output-dependent mechanism; the pathways responsible for increased MHC-I upon kinase inhibition were mapped. Activated mitogen-activated protein kinase (MAPK) signaling in mouse tumors in vivo suppressed components of MHC-I and the antigen presentation machinery. Translational relevance was shown by the improvement of therapeutic activity of TCR-mimic antibodies to MHC/peptide complexes. Druggable kinases may thus serve as immediately applicable targets for modulating immunotherapy for many diseases.

This Example is the first comprehensive analysis of kinase regulation of the human major histocompatibility complex class I (MHC-I), a central component of the CD8 T cell-mediated response. Efficient antigen presentation by MHC-I molecules on cancer cells is essential for T-cell based immunotherapies, including vaccines, checkpoint blockade, adoptive T-cell therapy, and TCR mimic antibodies. This Example provides a proof of concept using two druggable targets, EGFR and MEK. It is expected that these data can broadly influence translational and clinical trial design of targeted therapies combined with immunotherapy.

6.1.2. Methods:

Cell lines and culture conditions: After informed consent on Memorial Sloan-Kettering Cancer Center (MSK) Institutional Review Board—approved protocols, peripheral blood mononuclear cells (PBMCs) from HLA-typed healthy donors and patients were obtained by Ficoll density centrifugation. The sources for obtaining human mesothelioma cell lines are described previously (Dao et al., 2013, Sci Transl Med 5(176):176ra33). SKMEL5, PC9, and UACC257 were obtained from ATCC (Manasses, Va. USA). The NSCLC cell lines were obtained from the Scott Lowe laboratory. TPC1 cell line was a kind gift from the James Fagin lab. Cell lines were maintained in RPMI supplemented with 10% FBS and 2 mM L-glutamine unless otherwise mentioned. HEK293T were grown in Dulbecco's modified media with 10% FBS and 2 mM L-glutamine. Cells were checked regularly for mycoplasma.

ADCC: The HLA-A*02:01 positive mesothelioma cell lines JMN and Meso34, along with the melanoma cell line SK-MELS were used in the ADCC assay as a target (May et al. 2007, Clin Cancer Res 13:4547-4555) . Antibodies ESKM (Veomett et al., 2014, Clin Cancer Res 20(15):4036-4046), PRAME, or its isotype control hIgG1 at 3 ug/ml were incubated with target cells and fresh healthy donor PBMCs at different effector/target ratios for 6 hours, along with indicated doses of vehicle or trametinib in RPMI (Roswell Park Memorial Institute medium) supplemented with 10% fetal bovine serum (FBS). The supernatant was harvested, and the cytotoxicity was measured by a ⁵¹Cr release assay (Perkin Elmer).

Pooled RNAi screening: Briefly, a custom shRNA library targeting the full complement of 526 human kinases was designed using miR30-adapted DSIR (Designer of Small Interfering RNA) predictions refined with “sensor” rules (six shRNAs per gene) and constructed by PCR-cloning a pool of oligonucleotides synthesized on 12 k customized arrays (Agilent Technologies and CustomArray) as previously described, and discussed in depth below (Zuber et al., 2011, Nat Biotechnol 29(1):79-83). For validation, the LT3GEPIR shRNA vector was used (Fellmann et al., 2013, Cell Rep 5(6):1704-1713). Cells were transduced and selected with puromycin, then induced with 2 ug/ml doxycycline for 96 hours before evaluating levels of either BB7 (i.e., BB7.2), W6/32, ESK, or PRAME by flow cytometry.

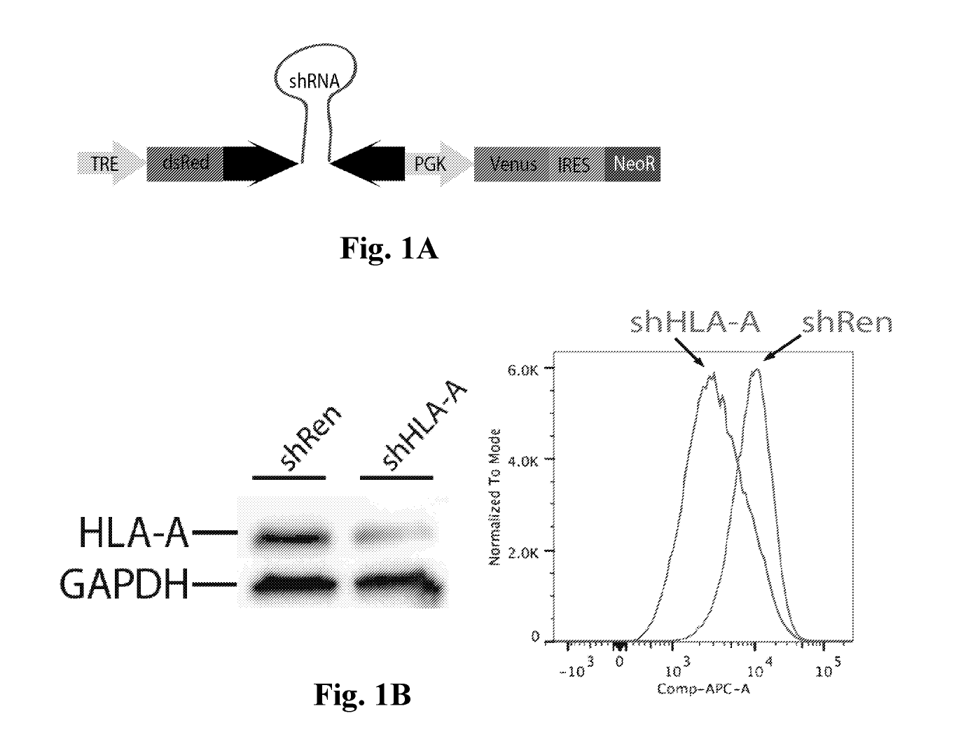

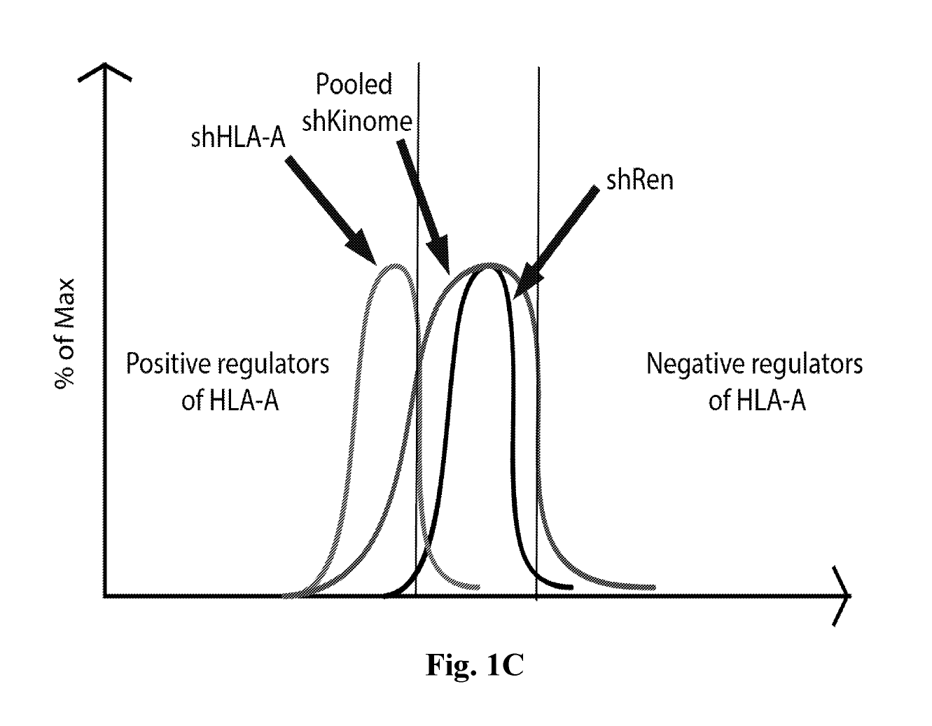

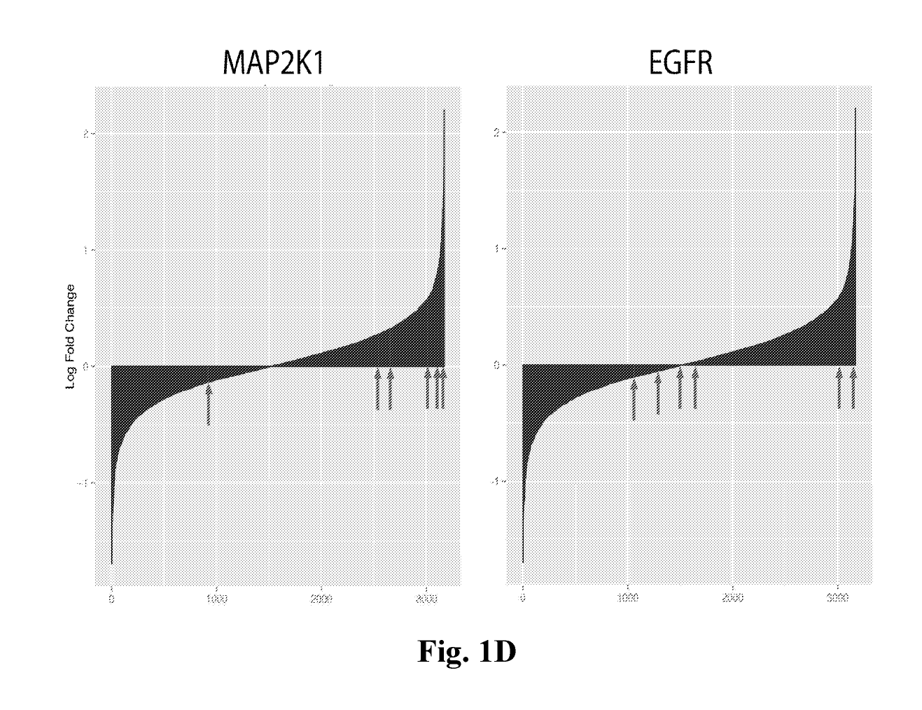

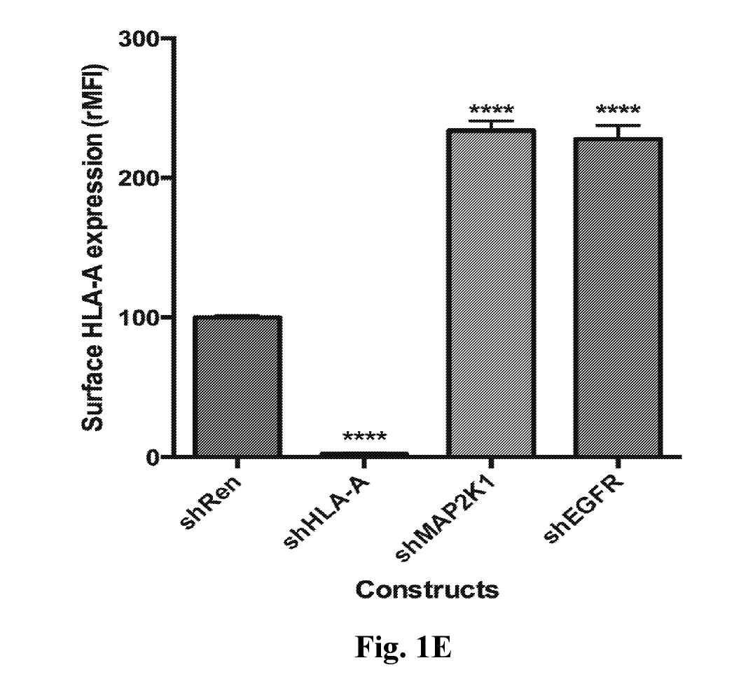

Specifically, the list of genes was obtained from KinBase Database (kinase.com/human/kinome/) and was manually curated. After sequence verification, 3156 shRNAs (5-6 per gene) were combined with positive control HLA-A—and negative-control Renilla targeting shRNAs at equal concentrations in one pool. JMN mesothelioma cells stably expressing the Tet-On rt-TA3 gene were used. This pool was subcloned into the TRMPV-Neo vector and transduced in triplicates into Tet-on JMN mesothelioma cancer cells using conditions that predominantly lead to a single retroviral integration and represent each shRNA in a calculated number of at least 1,000 cells. (FIG. 1A) Transduced cells were selected for 6 days using 1 mg ml⁻¹ G418 (Invitrogen); at each passage more than 30 million cells were maintained to preserve library representation throughout the experiment. After induction, T0 samples were obtained (˜30 million cells per replicate (n=3)) and cells were subsequently cultured in the presence of 2 μg ml ⁻¹ doxycycline to induce shRNA expression. After four days (Tf), about three million shRNA-expressing (dsRed⁺/Nenus⁺) cells were sorted for each replicate using a FACSAriaII (BD Biosciences). DAPI negative, dsRed+/Venus+ cells were sorted by FACS into three populations of BB7 low, BB7 middle, and BB7 high binding (FIG. 1). Genomic DNA from Tf samples was isolated by two rounds of phenol extraction using PhaseLock tubes (5 prime) followed by isopropanol precipitation. Deep-sequencing template libraries were generated by PCR amplification of shRNA guide strands as previously described (10). Libraries were analyzed on an Illumina Genome Analyzer at a final concentration of 8 pM; 50 nucleotides of the guide strand were sequenced using a custom primer (miR30EcoRISeq, TAGCCCCTTGAATTCCGAGGCAGTAGGCA). Hits with lower than 100 reads from the Illumina HiSeq were eliminated because they were not above background levels.

Relative representations of each individual shRNA were determined and compared in each given sorted population. Hits were separated phenotypically into negative regulators (the population one standard deviation below the mean fluorescence intensity) or positive regulators (the population one standard deviation above the mean fluorescence intensity) of HLA-A*02:01. The ratio of the shRNA ranking between the high and low population was compared, with a high ratio indicating a putative negative regulator of surface HLA-A*02:01. The scoring criteria for a gene being a negative regulator of HLA-A*02:01 was based on having 2 or more shRNA constructs score in the top 5% for fold difference in relative representation between BB7 high population and BB7 low population, with other constructs scoring within 1 SD of the mean fold change. The gene products with at least two shRNA sequences in the top 5% ratio were selected for further validation by other methods. The same discovery pipeline was used for identifying positive regulators of HLA-A*02:01.

Antibodies: Antibodies used for flow cytometry and western blots are listed in the supplemental materials section.

Real-Time PCR: Total RNA was extracted using Qiagen RNA Easy Plus (Qiagen; #74134) after cells were treated for 48 hrs with indicated inhibitor. RNA was converted into cDNA using gScript™ cDNA SuperMix (Quanta Biosciences Gaithersburg, Md. USA). Real-time assays were conducted using TaqMan real-time probes. (Life Technlogies) for human HLA-A (Hs01058806_gl), B2M (Hs00187842_ml), TAP1 (Hs00388677_ml), TAP2 (Hs00241060_ml), and TBP (Hs00427620_ml) using 50 ng cDNA. For assessment of gene expression using RT-PCR PerfeCTa® FastMix® II (Quanta), reactions were carried out in triplicates using standard thermocycling conditions (2 minutes at 50° C., 10 minutes at 95° C., 40 cycles of 15 seconds at 95° C., and 1 minute at 60° C.). TBP was used as internal control and ΔΔCT method was used for relative mRNA calculations.

Promoter based studies: GLuc luciferase promoter was obtained from Genecoepia (GeneCoepia Rockville, Md. USA) with the B2M promoter cloned upstream of the GLuc enzyme. Normalization was done to secreted embryonic alkaline phosphatase (SEAP) (under the constitutively active SV40 promoter). Cells were seeded at 5E3 cells/well and treated with indicated drugs for 72 hours. Luminescence quantitation was assayed using the Secrete-Pair Dual Luminescence Assay Kit (GeneCoepia Rockville, Md. USA).

Overexpression of B2M: Human B2M cDNA was cloned into the MSCV Puromycin vector.

Overexpression of mutant EGFR and NRAS: The pBABE retroviral vector encoding either EGFR harboring the L858R mutation was used to stably transduce H1299 cell line using HEK293T/Amphoteric cells and were selected in 2.5 ug/ml puromycin for 5 days. EGFR L858R can be obtained from Addgene (Addgene plasmid # 11012). For overexpression of NRAS, the pBABE NRAS Q61K plasmid was used to transduce H827 cells similar to as described above, and selected in 2 μg/ml puromycin. pBabe N-Ras 61K can be obtained from Addgene (Addgene plasmid # 12543).

Small molecule inhibitor studies: Compounds were obtained from SelleckChem (Houston, Tx. USA). Drugs were used at sub-cytostatic doses by titration using the Cell Titer Glo assay (Promega). All drugs were used in vitro at indicated doses in 1% dimethyl sulfoxide (DMSO). Experiments were performed at least twice with similar results, and data shown are representative.

siRNA knockdown: The JMN cell line was treated with a control scrambled small interfering RNA (siRNA), or siRNA against signal transducer and activator of transcription 1 (STAT1), STAT3, and RelA. Cells were treated with indicated drug 24 hours after siRNA knockdown for 72 hours before assaying for surface HLA-A by flow cytometry.

CC10/L858R microarray data:_{—} Expression data from tissue isolated from wild type (WT) and EGFR L858R transgenic mice were obtained from a previous study (GSE17373; www.ncbi.nlm.nih.gov/geo/query/acc.cgi?acc=GSE17373) and were selected for statistically significant data (p<0.05) for PDCD1(programmed cell death 1, also referred to as PD-1), CD274 (cluster of differentiation 274, also referred to as programmed death-ligand 1 (PD-L1)), TAP1 (transporter 1, ATP-binding cassette, sub-family B), TAP2 (transporter 2, ATP-binding cassette, sub-family B), H2-KD (H-2 class I histocompatibility antigen), and B2M (beta-2-microglobulin) gene expression between tumor bearing EGFR L858R lung tissue and normal lung tissue (Regales et al., 2009, J Clin Invest 119(10):3000-10, Akbay et al., 2013, Cancer Discov 3(12):1355-1363).

6.1.3. Results:

A pooled shRNA screen identified gene products regulating surface HLA-A*02:01. Loss or gain of function screens serve as starting points for identifying new regulators of protein expression and function. An shRNA library against the 550 currently annotated human kinases was used to perform a custom pooled screen. For each gene, six shRNA constructs were cloned into the TRMPV retroviral vector, a tetracycline regulated vector that couples a mir30 based shRNA to a red fluorescent protein, which allows easy tracking and sorting of cells productively expressing an shRNA (FIG. 1A) (Zuber et al., 2011, Nat Biotechnol 29(1):79-83). Knockdown of HLA-A*02:01 by use of an shRNA to this gene product in the same vector was tested as a positive control and caused strong knockdown by both western blot analysis and flow cytometry (FIG. 1B).

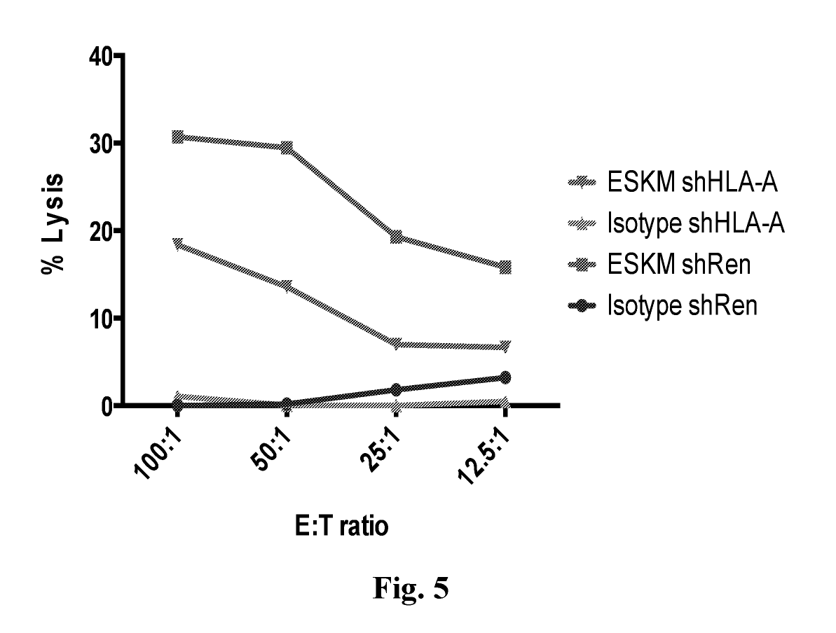

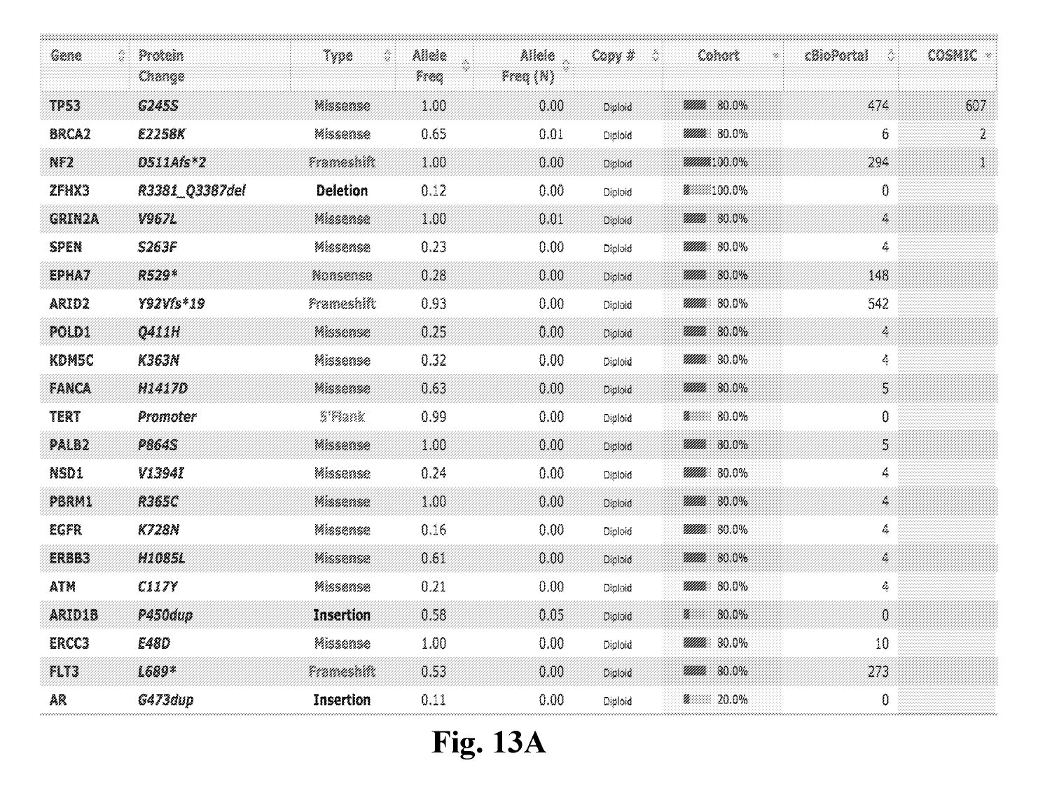

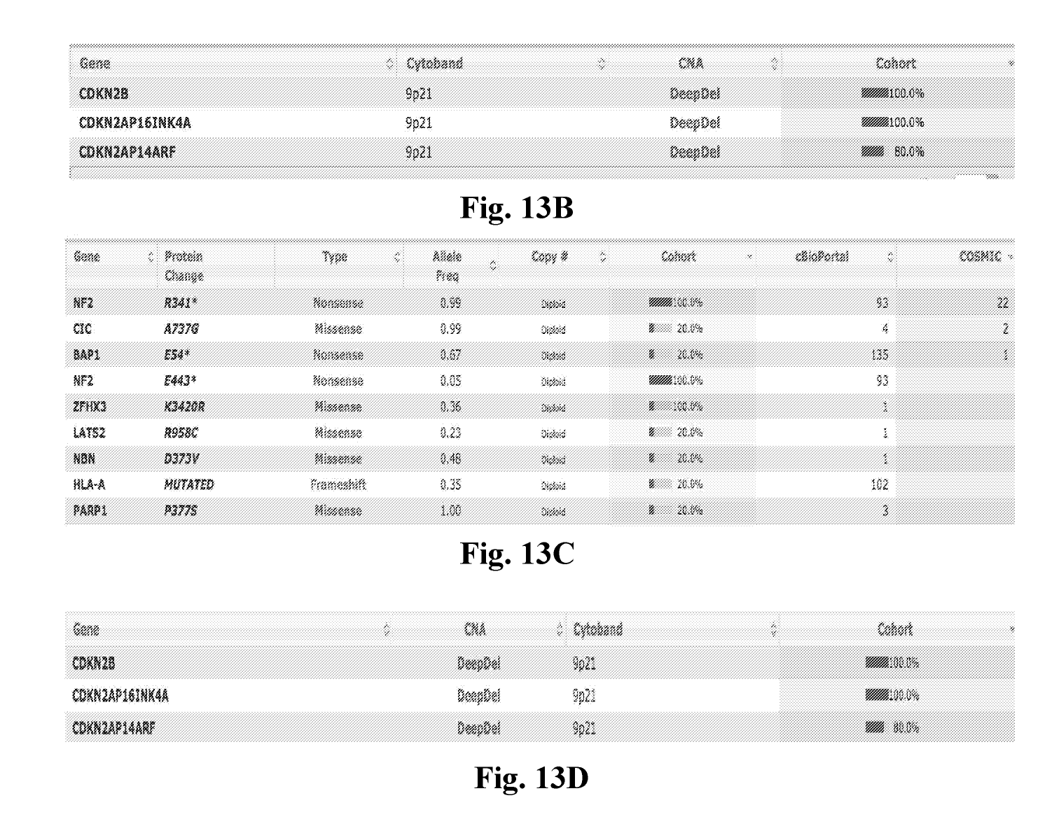

Previous work has shown levels of MHC-I and antigen presentation on surface HLA-A*02:01 to be an important determinant of efficacy for certain immunotherapies (Rivoltini et al., 1995, Cancer Res 55(14):3149-57). The human mesothelioma cell line JMN, which has stable levels of HLA-A*02:01 and which has been used as a target of MHC-I directed therapies in vitro and in vivo (Dao et al., 2013, Sci Transl Med 5(176):176ra33-176ra33) was utilized. Knockdown of HLA-A substantially decreased the killing efficacy of the T-cell receptor (TCR) mimic antibody ESK-M against the JMN mesothelioma cell line (FIG. 5). JMN was analyzed for presence of a pre-defined subset of mutations using the MSK IMPACT platform (FIG. 13A-FIG. 13D). No mutations or significant copy number alterations were observed in the HLA-A*02:01 or B2M genes.

The JMN cell line was screened with an shRNA library against the human kinome, as described above in Section 6.1.2, for genes acting as negative or positive regulators of surface HLA-A. Cell surface HLA-A was detected by flow cytometry with the HLA-A*02:01 specific antibody BB7.2 and fluorescence activated cell sorting was used to sort populations based on HLA expression (illustrated as in FIG. 1C). The top 5 hits are listed in Table 1

TABLE 1
Top 5 negative regulatory and top 5 positive regulatory kinase
genes that were hits from the screen tor regulating surface
HLA-A expression. Bold genes hav been validated. The percentile
ranking of the top 5% of shRNA constructs for each gene listed
is shown out of 3168 constructs tested.

| | | | | Negative or |
|---|---|---|---|---|
| | | Percentile Rank | Vali- | Positive |
| Genes | GeneID | of top shRNA | dated | Regulatory |
| GRK7 (G | 131890 | 99.8%, 98.7% | Yes | Negative |
| protein-coupled | | | | |
| receptor kinase 7) | | | | |
| MAP2K1 | 5604 | 99.7%, 98.1%, | Yes | Negative |
| (mitogen- | | 95.1% | | |
| activated protein | | | | |
| kinase kinase 1) | | | | |
| EGFR | 1956 | 99.5%, 95.3% | Yes | Negative |
| (epidermal | | | | |
| growth factor | | | | |
| receptor) | | | | |
| RET (ret proto- | 5979 | 99.3%, 95.8% | Yes | Negative |
| oncogene) | | | | |
| BRSK1 (BR | 84446 | 99.3%, 97.3% | Yes | Negative |
| serine/threonine | | | | |
| kinase 1) | | | | |
| DAPK3 (death- | 1613 | 0.22%, 1.1% | No | Positive |
| associated | | | | |
| protein kinase 3) | | | | |
| DDR2 (discoidin | 4921 | 0.28%, 2.8% | Yes | Positive |
| domain receptor | | | | |
| tyrosine receptor | | | | |
| kinase 2) | | | | |
| CDK7 (cyclin- | 1022 | 0.41%, 3.3% | Yes | Positive |
| dependent kinase 7) | | | | |
| MINK1 | 50488 | 0.47%, 2.1% | Yes | Positive |
| (Misshapen-Like | | | | |
| Kinase 1) | | | | |
| MAPK3 | 5595 | 0.51%, 4.9% | No | Positive |
| (Mitogen- | | | | |
| Activated Protein | | | | |
| Kinase 3) | | | | |

Based on this analysis, MAP2K1 and EGFR were identified as negative regulators of surface HLA-A*02:01. EGFR and MEK were chosen for further investigation because of the availability of clinically approved drugs targeting these kinases both in non-small cell lung cancer (NSCLC) and metastatic melanoma respectively (Flaherty et al., 2012, N Engl J Med 367(2):107-114), as well as extensive use of immunotherapy.

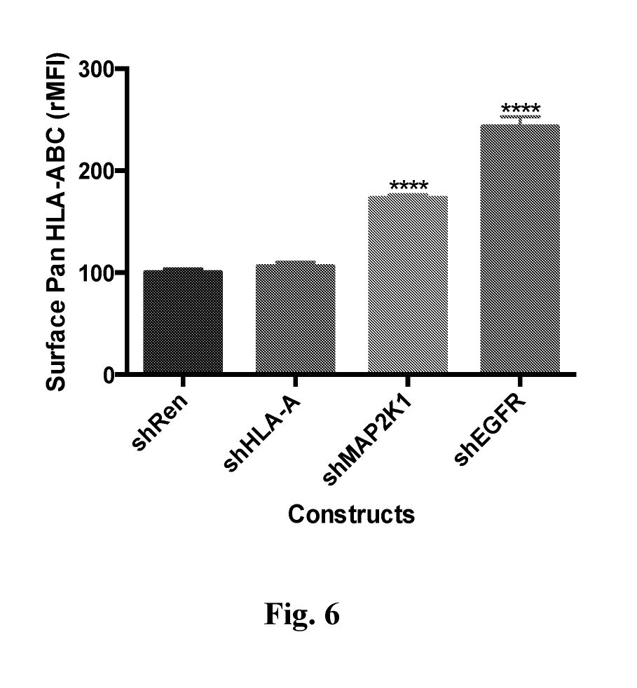

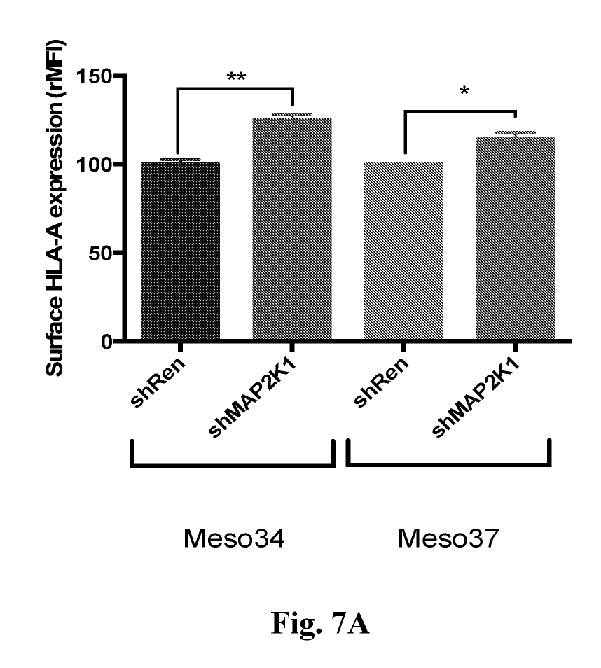

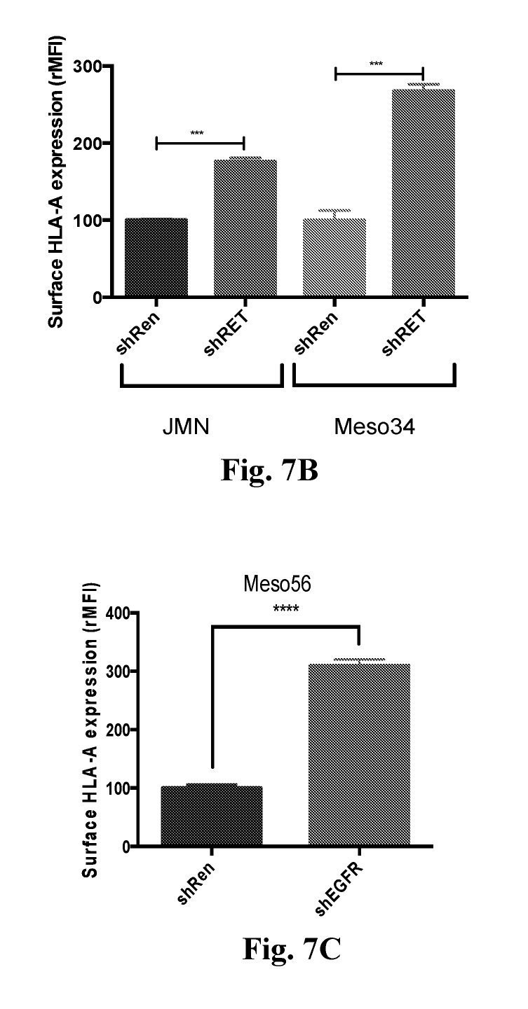

EGFR is a receptor tyrosine kinase that binds epidermal growth factor and is frequently found to be activated by mutation in NSCLC. Activated EGFR signals through multiple downstream pathways, including the MAPK pathway. shRNA constructs against MAP2K1 and EGFR showed a large increase in relative representation in the BB7 high sorted population versus the BB7 low population, indicative of a negative regulator of HLA-A*02:01 surface expression (FIG. 1D). Each of these genes was validated with independent shRNA knockdown to the gene products and significant increases in HLA-A*02:01 by flow cytometry (FIG. 1E) were seen. Interestingly, these effects were seen not only with HLA-A*02:01, but with total HLA-A, B, and C, suggesting coordinated control of all HLA surface expression as measured using the W6/32 mAb as well (FIG. 6). These findings were reproduced in multiple mesothelioma cell lines (FIG. 7A). The RET protooncogene was also identified as potential target, but was not further studied at this time because no inhibitor of adequate specificity was available (FIG. 7C).

Examples of positive genetic regulators of HLA-A, including two putative positive regulators DDR2 and MINK1 (Table 1; FIG. 7A, FIG. 7B), were identified and their activities were confirmed as well using siRNA knockdown (FIG. 7C). Therefore, the kinase screen was able to discover multiple positive and negative regulators of HLA expression, each of which, in principle, could be explored further for mechanism and clinical utility. The top 5 negative regulators evaluated were confirmed by additional study, whereas 3/5 of the positive regulators were validated (Table 1).

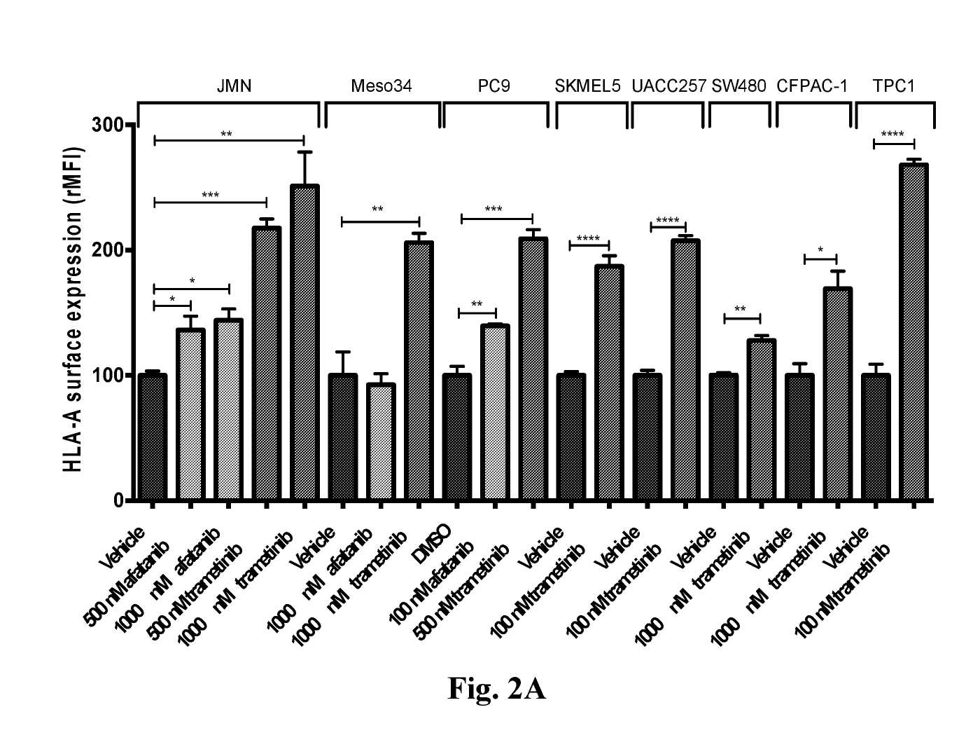

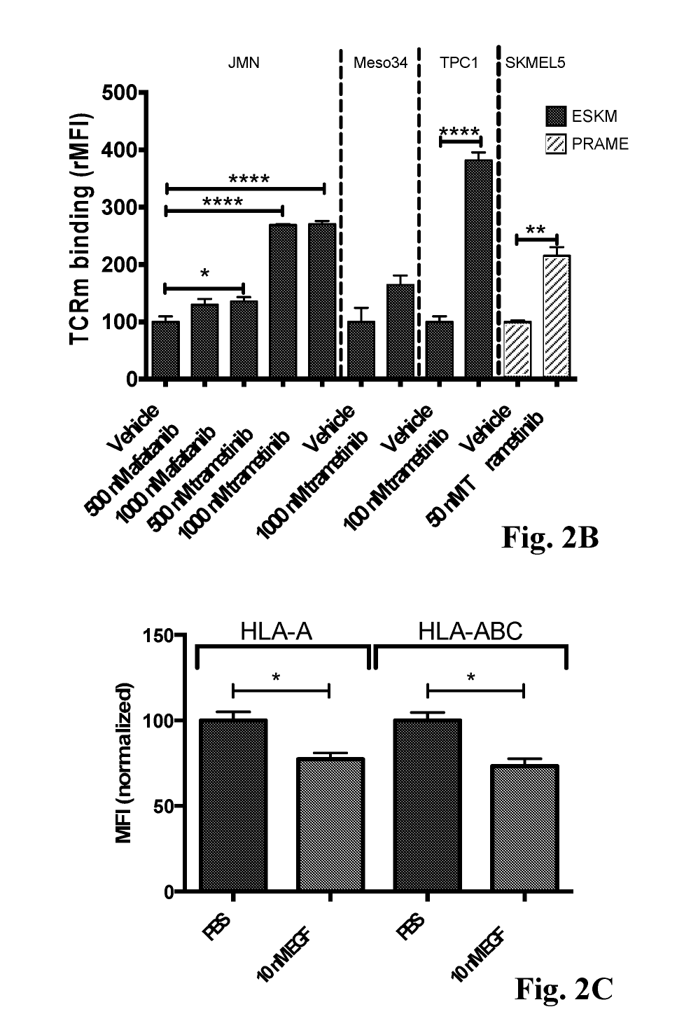

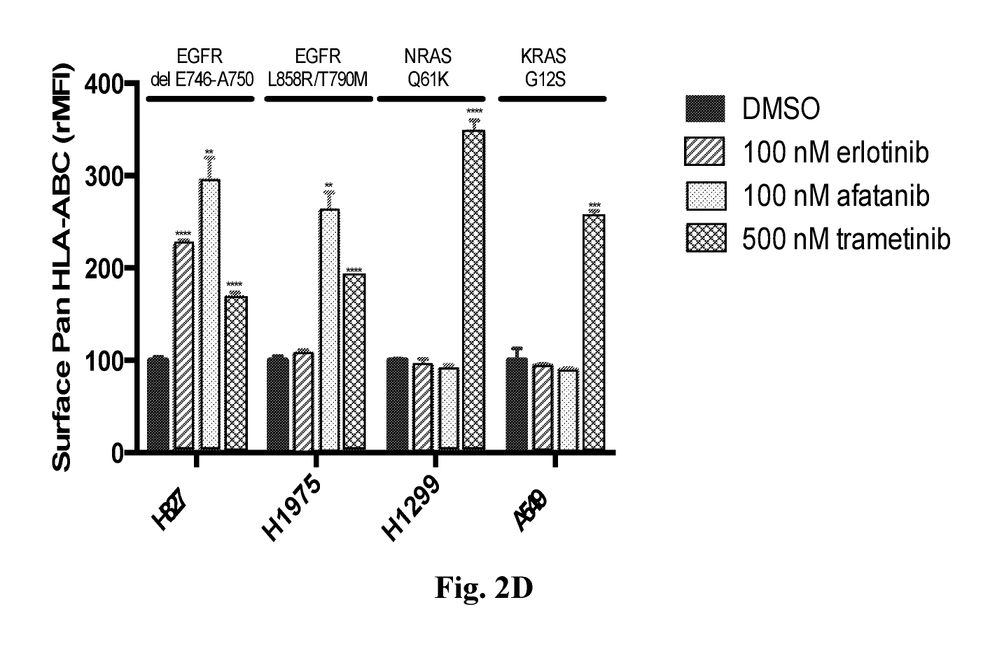

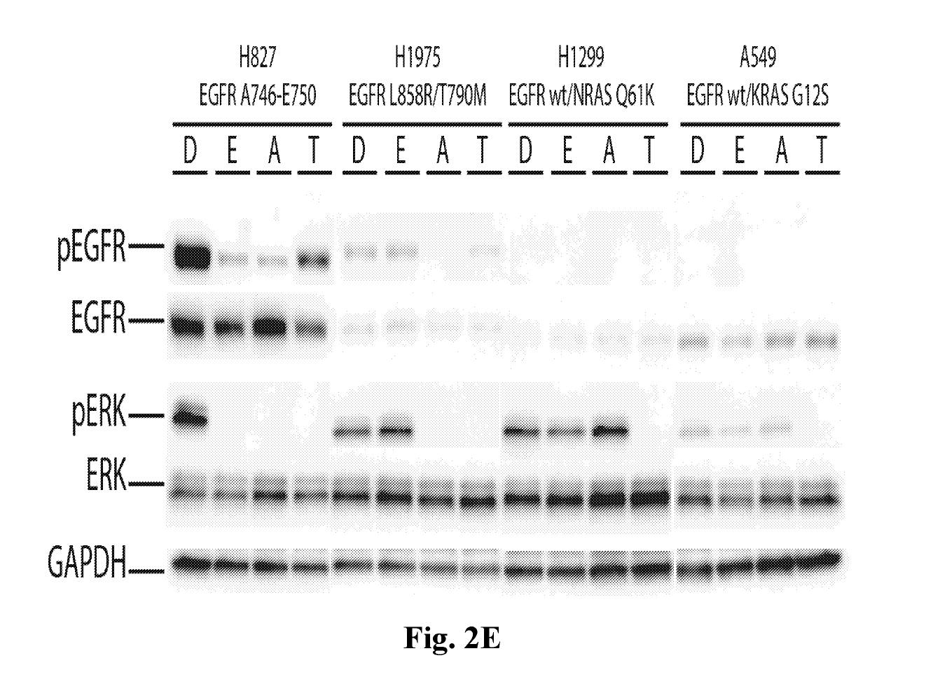

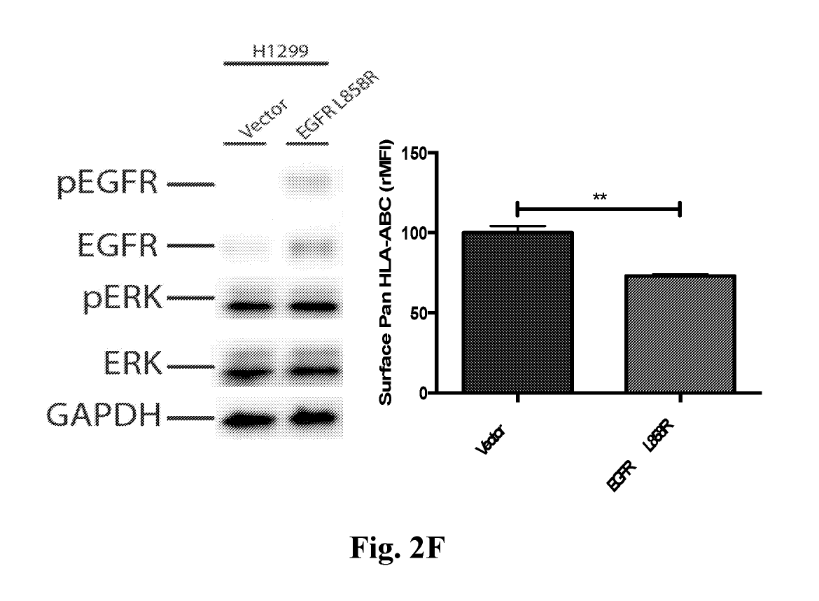

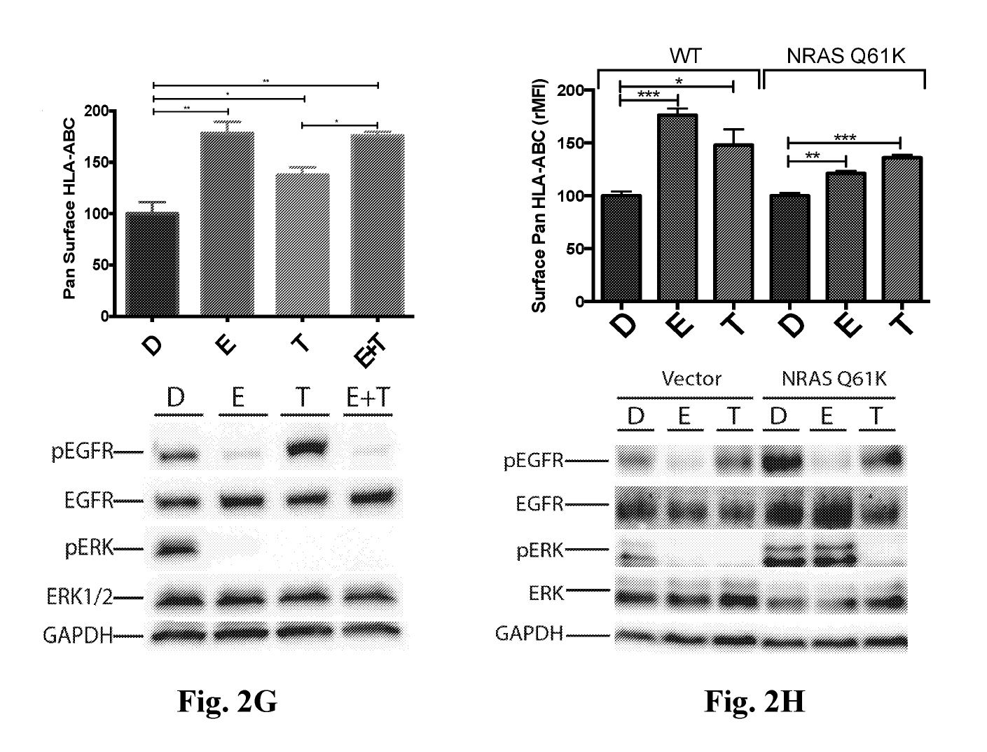

Multiple potent small molecule inhibitors exist for EGFR and MEK, with several already FDA approved, and others currently in clinical trials for various cancers (Flaherty et al., 2012, N Engl J Med 367(2):107-114). Of note, the initial screen was performed in a cell line with EGFR activation and an identified EGFR mutation (FIG. 13) (Brevet M et al., 2011, J Thorac Oncol 6(5):864-874). In multiple cell lines, the ability of inhibitors to phenocopy the loss of kinase expression leading to increased HLA-A expression seen with shRNA was tested. Cell surface HLA-A*02:01 levels increased in response to MEK inhibition for 72 hours with the selective MEK inhibitor trametinib in mesothelioma cell lines with activated MAP kinase signaling (FIG. 2A). JMN and PC9, a NSCLC cell line with an activating EGFR mutation (del E746-A750), responded to the EGFR inhibitor afatanib, while the Meso34 cell line without an EGFR mutation did not respond to afatanib at the same dose, demonstrating selectivity for activation mutations in the MAPK pathway leading to a response to HLA-A up-regulation (FIG. 13). An effect of MAP kinase pathway inhibition on up-regulation of HLA-A in the context of gain of function mutations or activation of other targets in the MAP kinase pathway, such as the KRAS G12V mutation in SW480 and CFPAC-1, the RET/PTC1 gene rearrangement in TPC1 thyroid cell line, and the BRAF V600E mutation seen in the UACC257 and SK-MEL-5 melanoma cell lines, was detected (FIG. 2A). The MEK inhibitor (MEKi) trametinib did not affect surface HLA-A expression on normal PBMC cells, showing that this effect is specifically seen in cells with activated signaling.

To confirm that the increased HLA expression on the cell surface had important functional significance, it was investigated whether the increased HLA-A*02:01 resulted in enhanced antigen presentation. For this purpose, the MHC/peptide density was quantified by use of TCR mimic mAb selective for two well-validated tumor associated epitopes presented by HLA-A*02:01, WT1 peptide and PRAME 300 peptide (Chang et al., 2015, American Society of Hematology, Available at: ash.confex.com/ash/2015/webprogram/Paper82235.html, Krug et al., 2010, Cancer Immunol Immunother 59(10):1467-79). Consistent with the increased surface HLA-A*02:01 expression, increased binding of the two TCR-mimic antibodies was also observed upon inhibition of MEK and EGFR (FIG. 2B).

The regulatory activity of the pathway was confirmed in a gain of function experiment by further stimulating the ERK pathway with epidermal growth factor (EGF). EGF binding to EGFR suppressed surface HLA-A and HLA-A,B,C, providing additional confirmation of the importance of the MAPK pathway in regulating surface MHC (FIG. 2C).

The mechanism by which the MAP kinase pathway suppresses levels of HLA-A is unknown. Given that many cancers have activating mutations in specific genes in the MAP kinase pathway, inhibition of the identified hits was investigated in cell lines harboring mutations in EGFR, or downstream in Ras. A panel of NSCLC cell lines with activating mutations in EGFR, such as the delE746-A750 in H827, or L858R/T790M mutation in H1975, was utilized. The delE746-A750 confers sensitivity to erlotinib, whereas the T790M confers resistance to erlotinib and other first generation EGFR inhibitors, but is sensitive to afatanib (Cross et al., 2014, Cancer Discov 4(9):1046-61). EGFR wild type NSCLC lines with downstream mutations, such as activating NRAS Q61K in H1299 or KRAS G12S in A549, were also utilized.

Use of the EGFRi erlotinib and afatanib up-regulated surface MHC-I if the cell line had the sensitizing mutation, whereas all responded to trametinib MEKi (FIGS. 2D and 2E). The sensitivity to EGFRi erlotinib and afatanib up-regulating surface MHC-I was not observed with downstream activating RAS mutations. Expression of the activating EGFR mutation L858R suppressed MHC-I in H1299 NRAS Q61K mutant cell lines (FIG. 2F).

Interestingly, H827 responded more strongly to EGFRi by erlotinib than MEKi by trametinib, despite a similar level of suppression of pERK, a downstream marker of MEK activity. The combination of MEKi and EGFRi was equivalent to EGFRi alone. (FIG. 2G). The NRAS Q61K mutation, shown to cause resistance to EGFRi and persistent activation of the MAPK pathway in H827, was introduced. Use of the EGFRi still had an effect on surface MHC-I despite no change in pERK output on the H827 NRAS Q61K cell line (FIG. 2H). This could have been due to activation of parallel signaling pathways in EGFR mutant cancers or differential stimulation of ERK. These data suggest that MAPK pathway was not the only determinant of EGFR mediated regulation of surface MHC-I. Given that both EGFR and MEK are involved in signaling via the MAP kinase pathway, these data validated the importance of this pathway in regulating surface HLA-A and MHC-I.

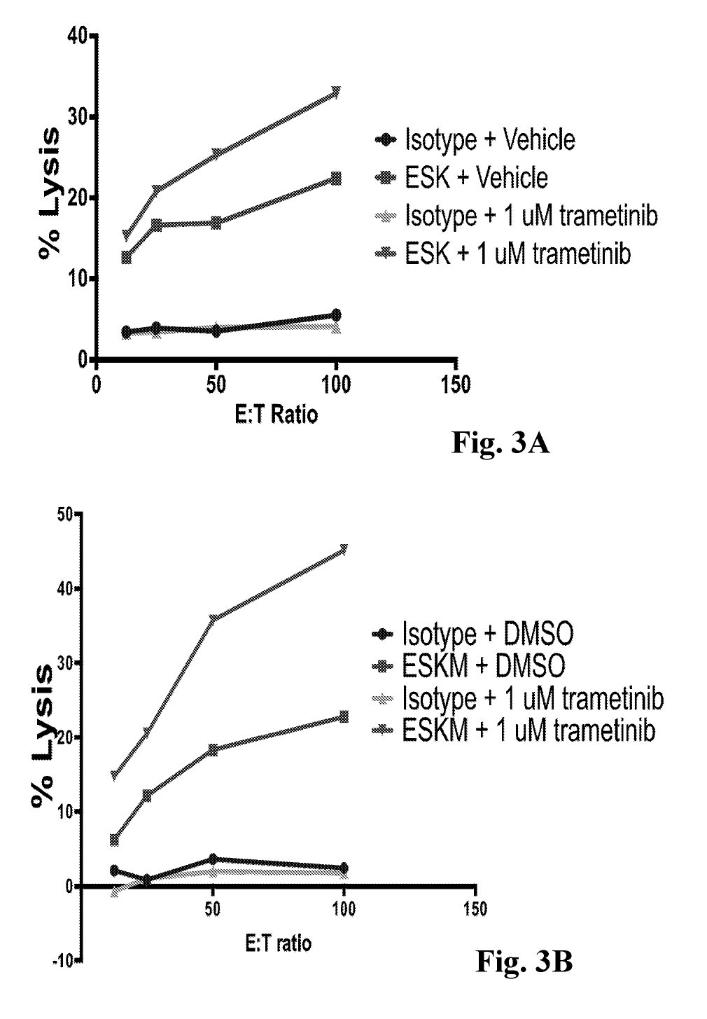

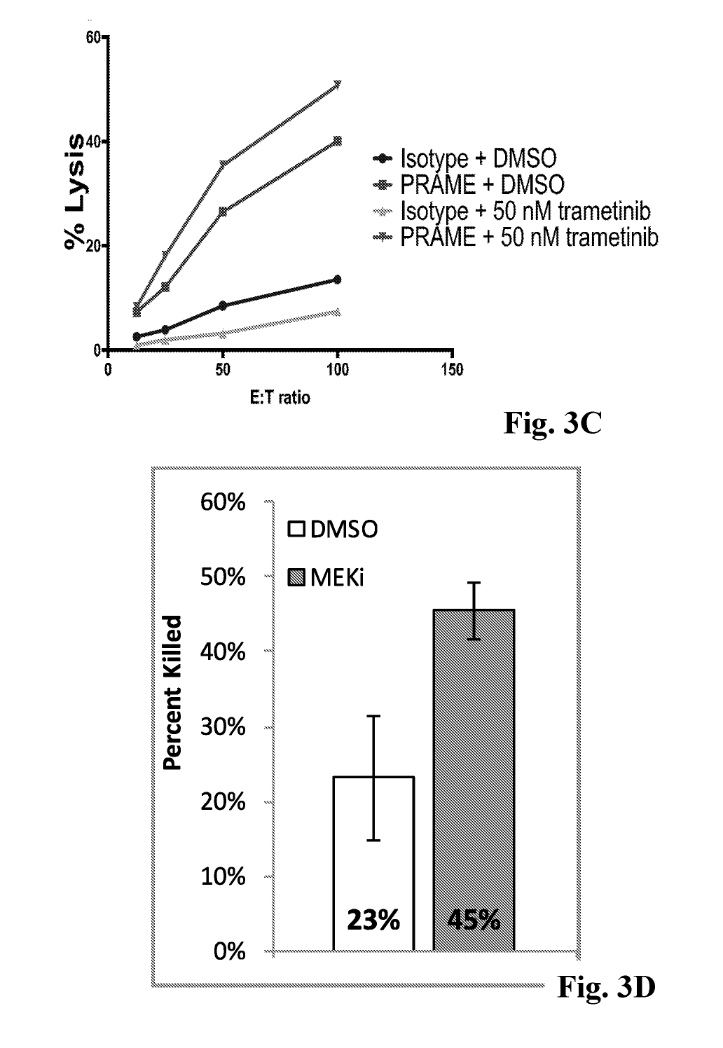

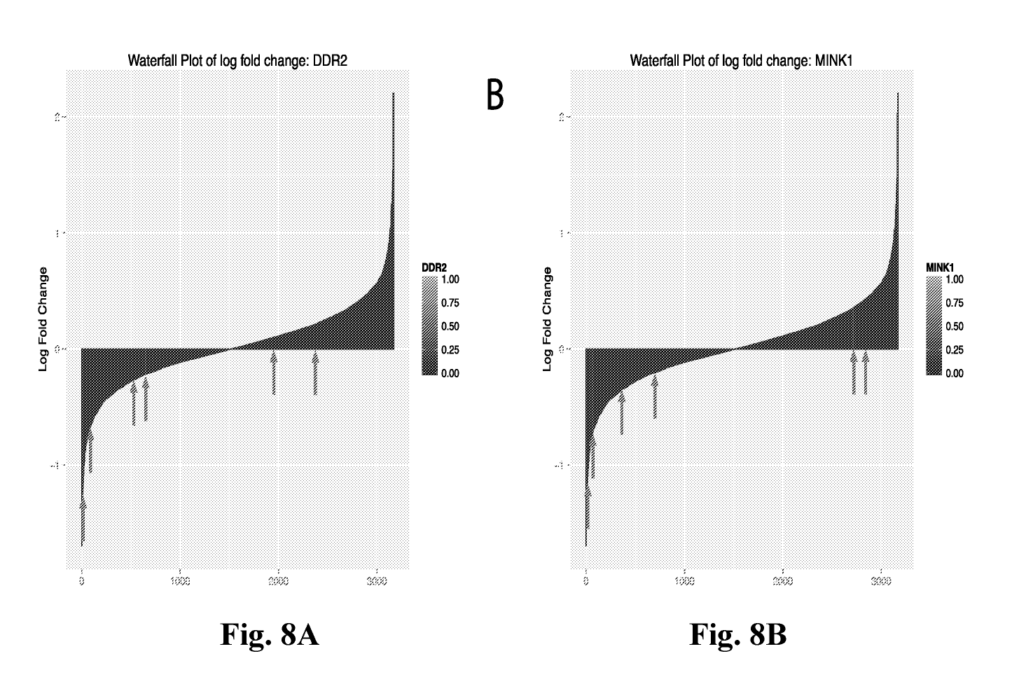

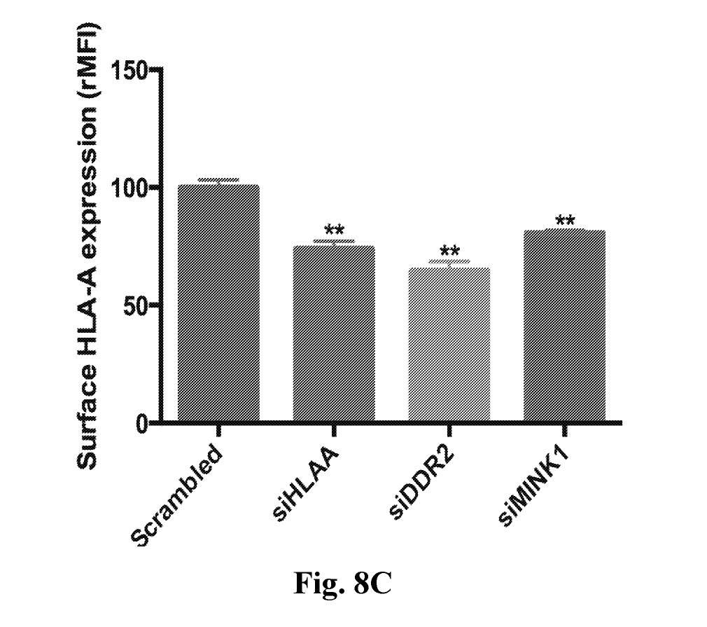

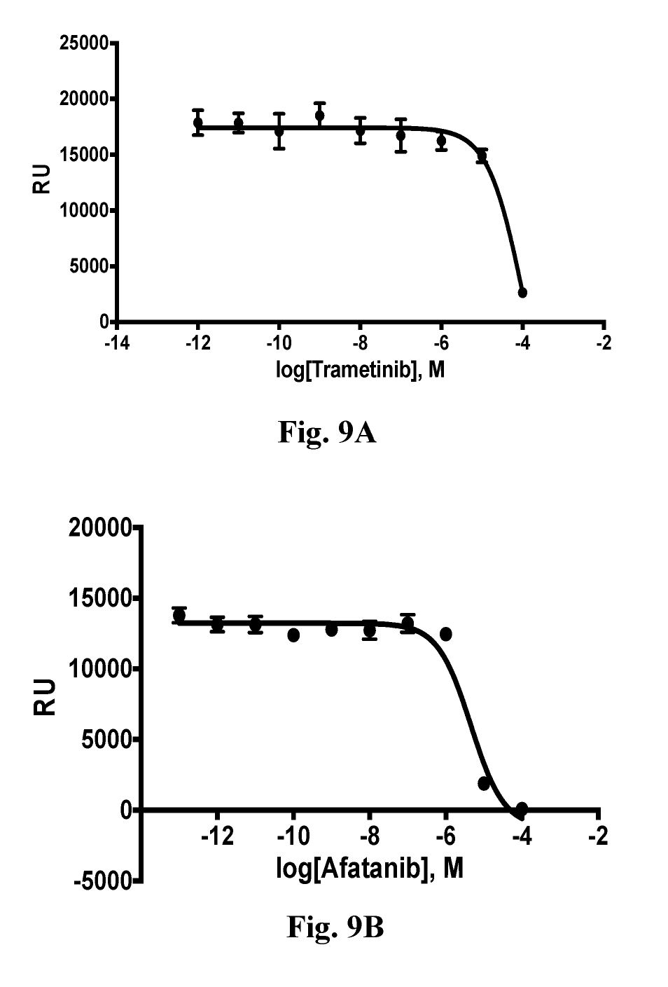

The effects of modulating HLA-A*02:01 levels on the efficacy of an immunotherapy that depends on HLA-A*02:01 upregulation were tested next. The TCR mimic antibody ESKM, which targets a peptide from WT1 in the context of HLA-A*02:01, and Pr20m, which targets a peptide from PRAME, were used as models for immunotherapies which are specifically dependent on HLA-A*02:01 expression. The cytotoxicity of ESKM against the JMN and Meso34 human mesothelioma cell lines was increased by MEK inhibition with trametinib (FIGS. 3A 3B), which was used at a non-cytotoxic dose (FIG. 8). This was also observed with use of a MEKi trametinib in SK-MEL-5 human melanoma cell line with the Pr20m mAb that recognizes a peptide from the PRAME protein presented on HLA-A*02:01, further demonstrating this observation in multiple targets in multiple cell lines (FIG. 3C). The increased killing was also validated using MEKi to up-regulate MHC-I with the pmel-1 gp100 positive B16F10 mouse melanoma cell line with effector pmel-1 TCR T-cells from transgenic lymphocytes (FIG. 3D). The MEKi was tested alone (without using the TCR T-cells) and gave the baseline killing, which was subtracted from the percentage killed presented in FIG. 3D.

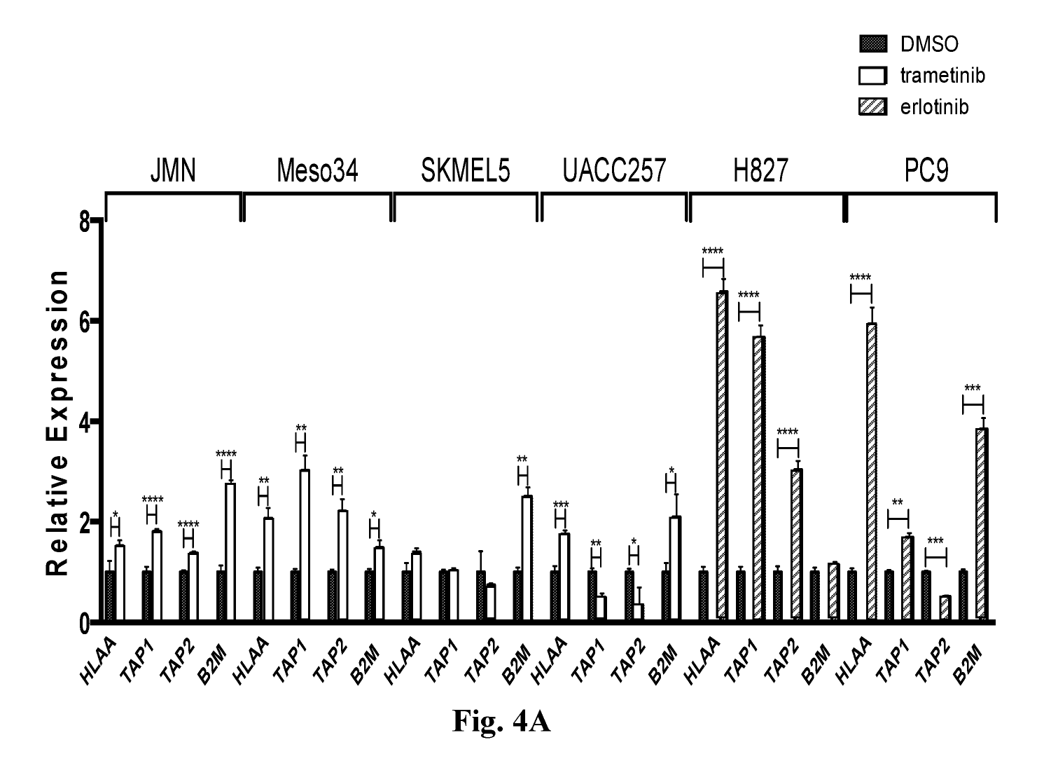

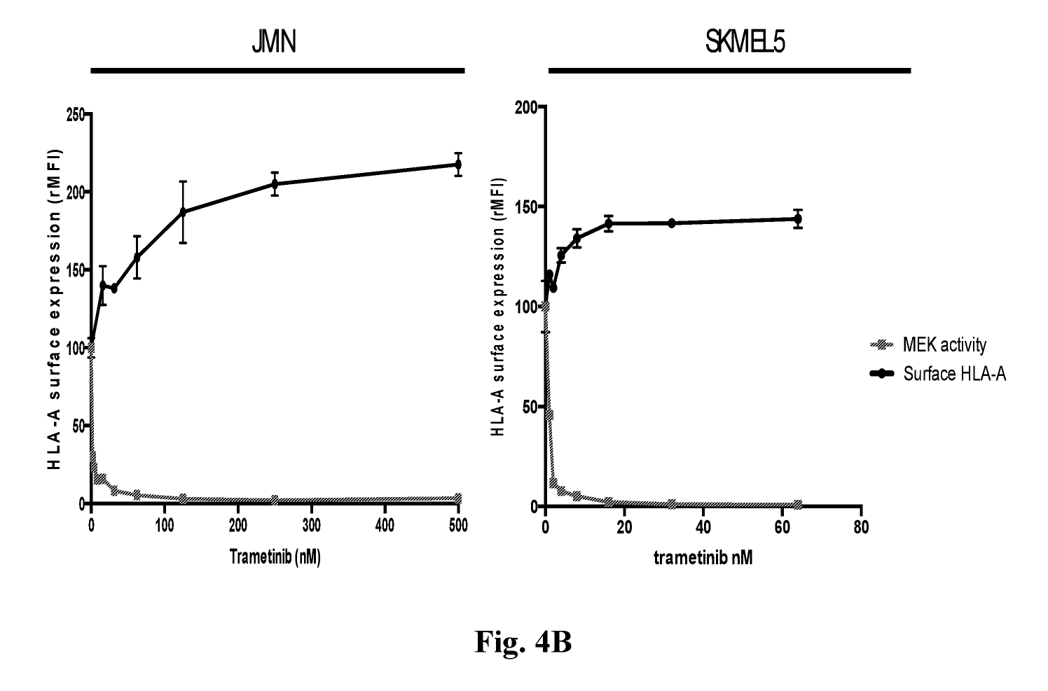

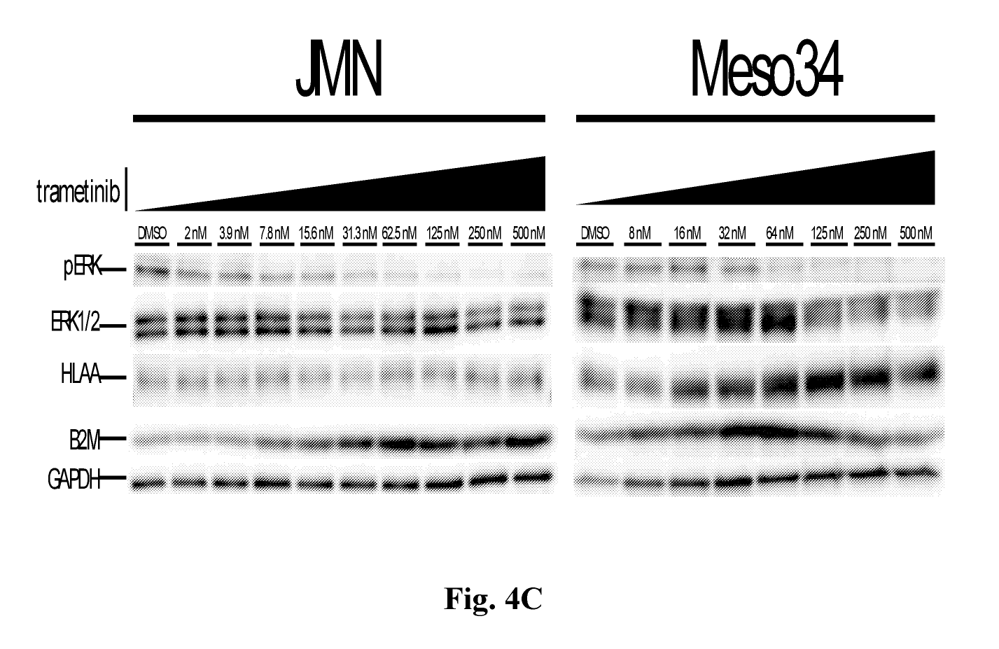

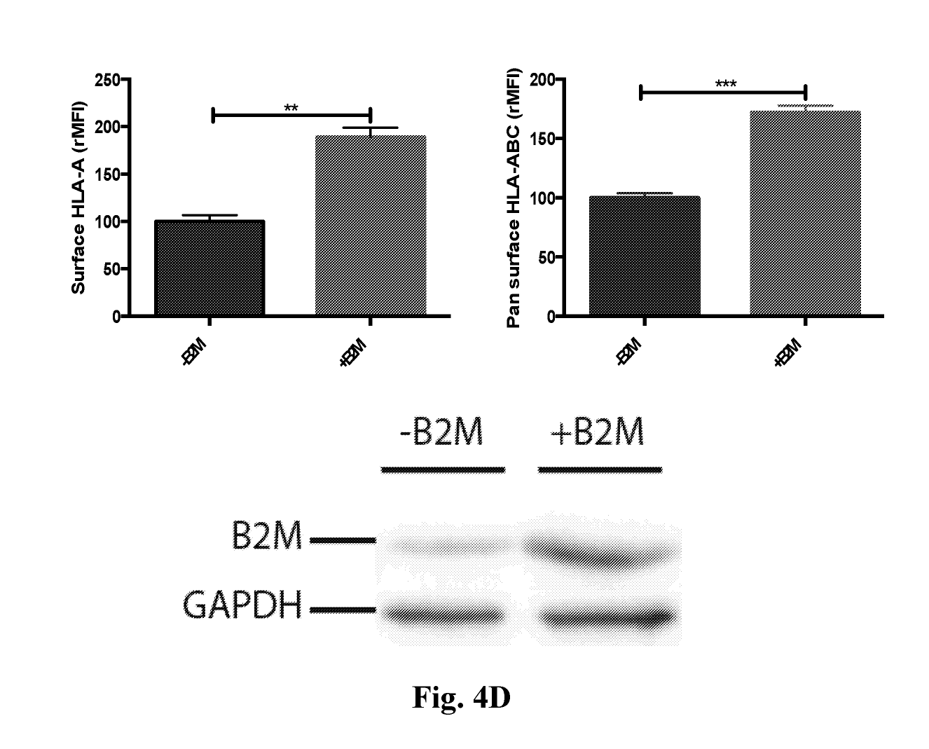

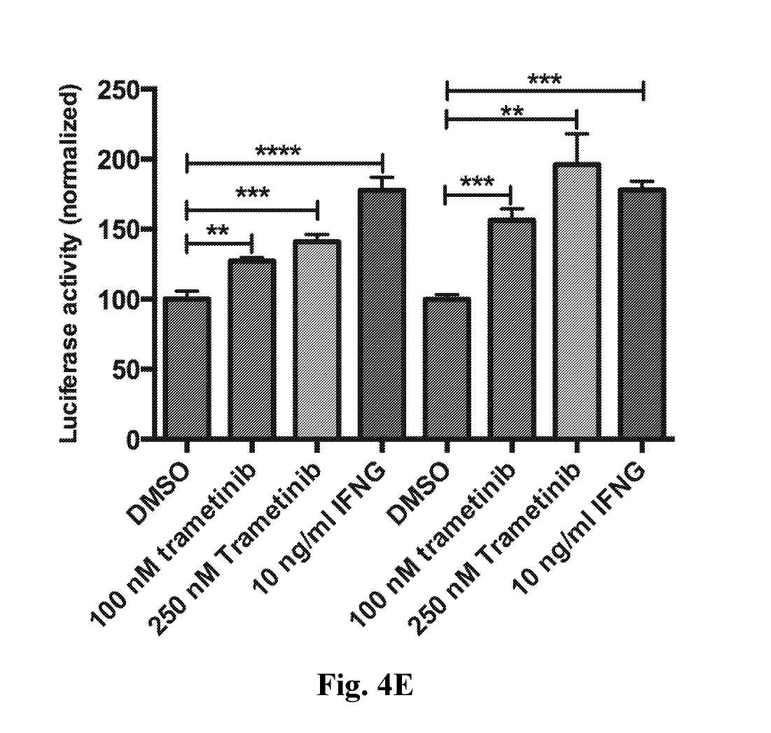

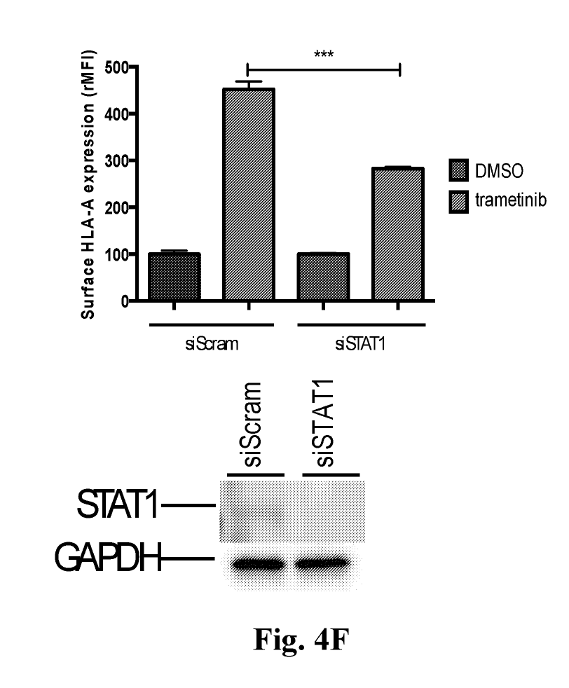

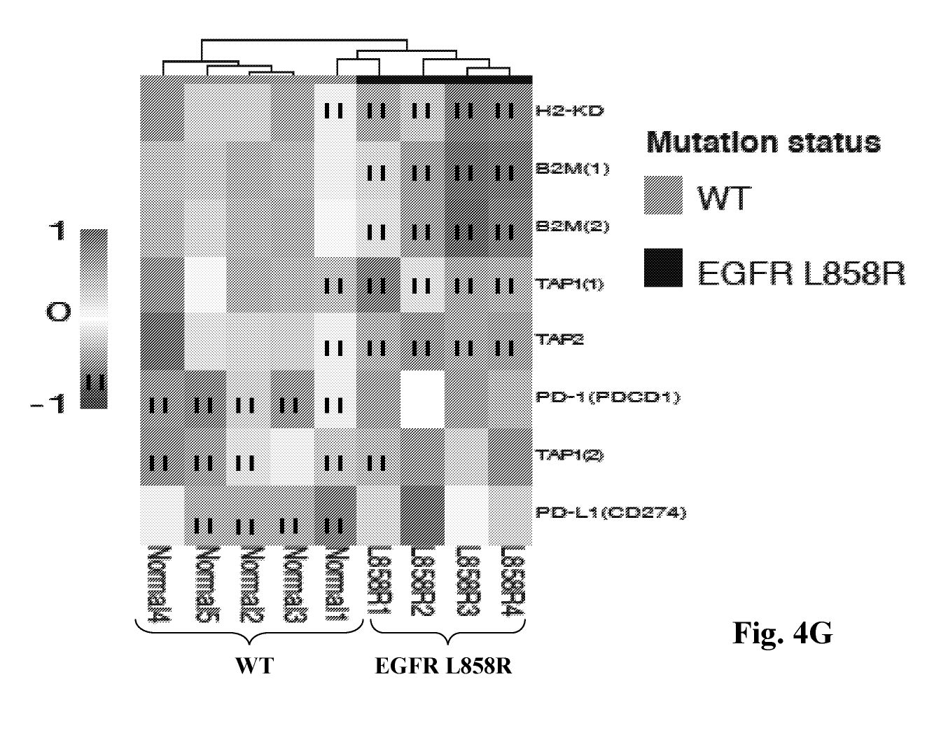

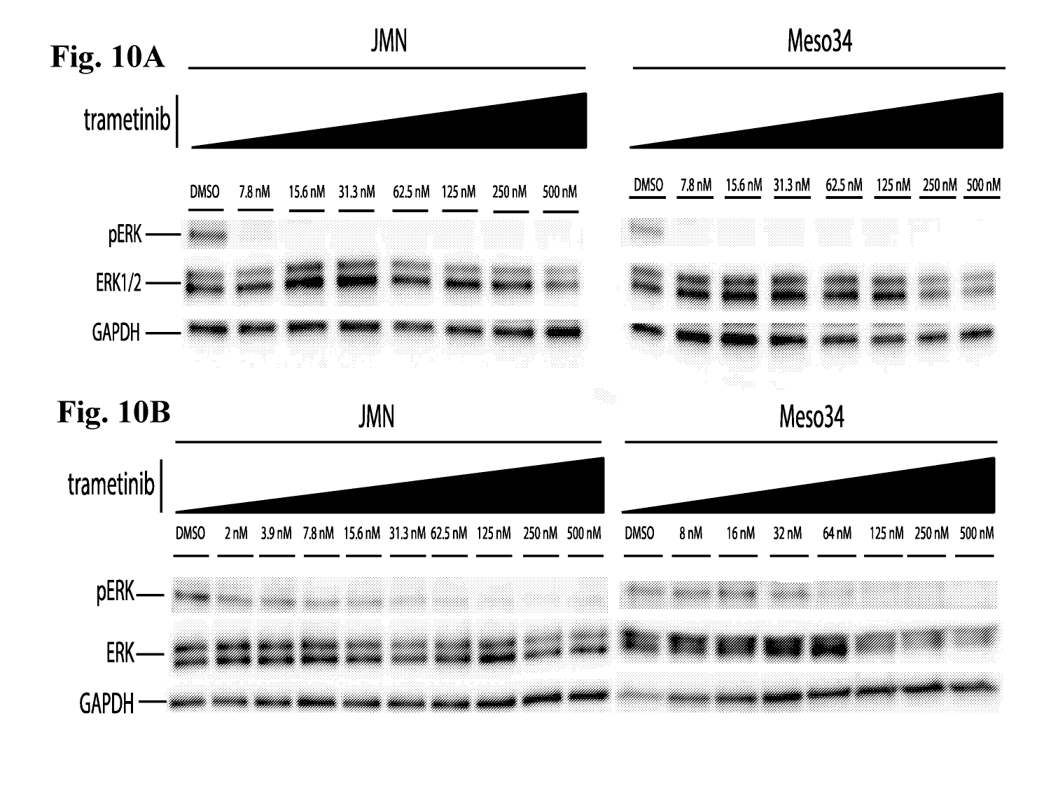

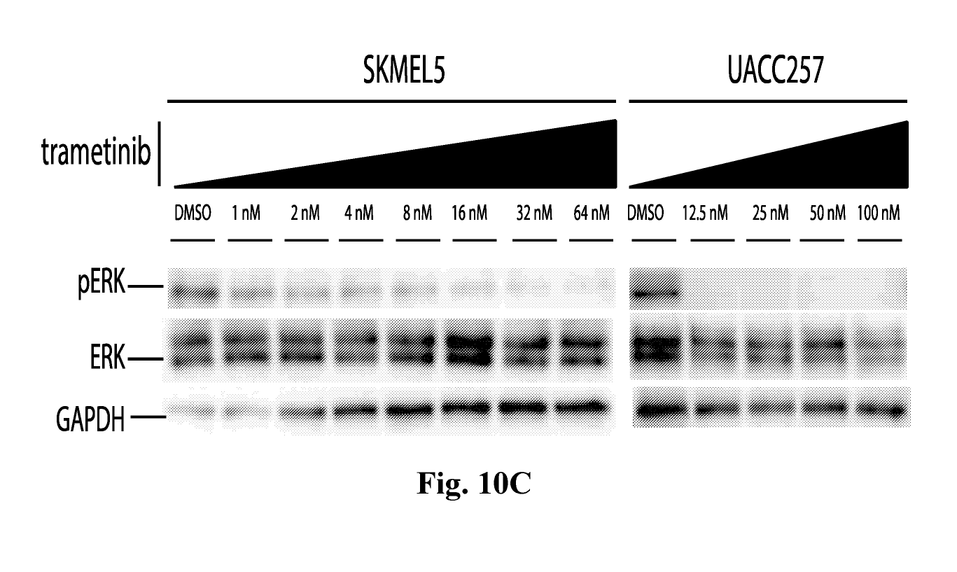

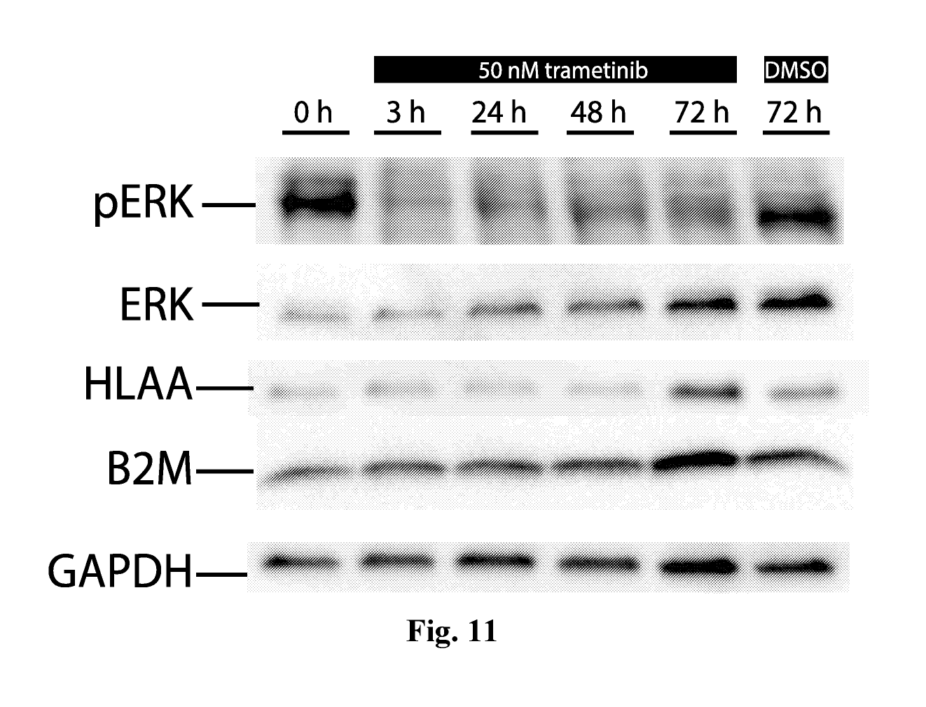

It was hypothesized that the inhibition of the MAP kinase pathway might act on other components of the antigen presentation machinery in addition to MHC-I molecules, thus allowing increased epitope expression in the more abundant cell surface HLA molecules. Indeed, EGFR and MEK inhibition produced an increase in mRNA gene expression of HLA-A along with other key components of the antigen presentation pathway and MHC-I structure, as TAP1, TAP2, B2M, (FIG. 4A). Doses of trametinib were chosen over the inhibition concentration (IC50) of MEK by using phospho-ERK (pERK) as a readout of MEK inhibition at 72 hours (FIGS. 10B and 10C). The JMN and Meso34 cells at time=1 hour were sensitive to trametinib at doses less than 10 nM as previously reported, but required higher doses to sustain inhibition of pERK at time=72 hours due to strong feedback (FIG. 10A). These data correlated with previous findings that BRAF mutant cell lines are the most sensitive to MEK inhibition, when compared to BRAF wild type cell lines harboring further upstream mutations (FIG. 10C)(Solit et al., 2006, Nature 439(7074):358-362). A time course showed maximal inhibition of MEK at 3 hours, with maximal increases of HLA-A and B2M at 72 hours (FIG. 11). Surface HLA-A increased in a dose dependent manner with increasing MEK inhibition in both melanoma and mesothelioma (FIG. 4B). The phenotypes observed were unlikely from off target effects of the drug given the dose response observed on pERK levels and plateau of the dose response of surface HLA-A observed.

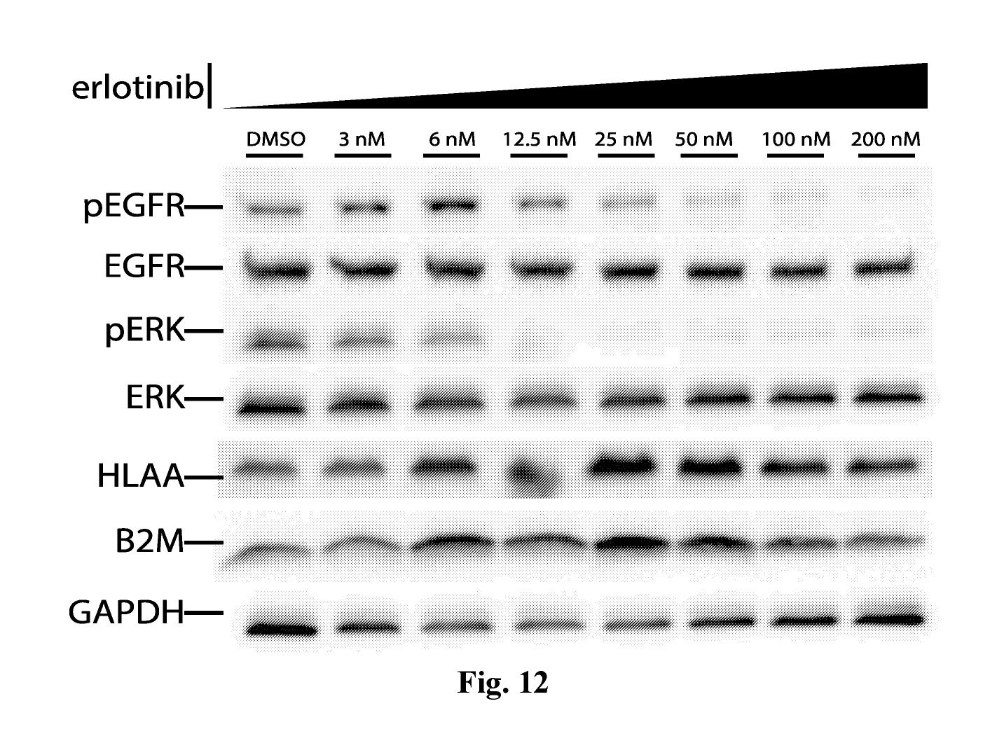

Antibodies against pERK, a downstream target of MEK, along with total ERK1/2 were used to show dose responsive increases in response to trametinib in inverse correlation with HLA-A protein levels. Interestingly, the levels of B2M increased much more than HLA complexes in multiple cell lines, consistent with the gene expression data (FIG. 4C). EGFRi with erlotinib also caused a dose dependent increase in HLA-A and B2M (FIG. 12). Because B2M is required for surface presentation of HLA, A,B, C and stability of the MHC-I molecules on the cell surface, the potential role of B2M in controlling cell surface HLA-A expression was investigated. Overexpression of B2M produced an increase in cell surface HLA-A levels and pan HLA-ABC surface levels, phenocopying the effect of MEK inhibition (FIG. 4D). The mechanism of up-regulation of HLA-A and B2M upon MEK inhibition was further investigated. The HLA-A and B2M gene expression is regulated by multiple regulatory domains in the promoter region, including the ISRE site, E box, and NF-κB sites. Using a luciferase based promoter assay, it was demonstrated that upon MEKi, an increase in activity on the HLA-A and B2M promoter was observed in a dose dependent manner (FIG. 4E).

Knockdown of the STAT1, STAT3, and RelA (component of NFκB complex) were performed on JMN cells, along with treatment with MEKi. STAT1 knockdown had the largest effect in blunting up-regulation of surface HLA-A after MEKi, suggesting a role for STAT1 in response to MEKi (FIG. 4F).

Microarray profiling of the lung bearing tumors from transgenic EGFR L858R, which activates the MAPK pathway, compared to normal lungs, demonstrated suppression of mouse MHC-I and antigen presentation components H2-KD, and B2M, thereby confirming the effects of this pathway in vivo (FIG. 4G) (Regales et al., 2009, J Clin Invest 119(10):3000-10). Up-regulation of PD-1 and PD-L1 markers in the tumors was also observed as previously published (Akbay et al., 2013, Cancer Discov 3(12):1355-1363).

6.1.4. Discussion

Immunotherapy of cancer is emerging as a successful and important component of treatment. MHC molecules presenting antigens are the target of multiple therapeutic strategies that involve vaccines, T cells or TCR's, TCR mimic antibodies, or T cell checkpoint blockade. The latter, a highly effective recent example in cancer therapy, appears to require presentation of neoantigens on MHC-I on the surface of cancer cells (Rizvi et al., 2015, Science (80−) 348(6230):124-128, Snyder et al., 2014, N Engl J Med 371(23):2189-2199, Van Allen et al., 2015, Science (80-) 350(6257):207-211). Many immunotherapies have focused on intrinsic effector cell mechanisms, such as the T-cells, for modulating the immune response, but not the target of the immune attack. The ability to regulate such responses by affecting target cells could have an important impact on both disease and therapy. It is proposed that kinases are a readily druggable pathway that might be used in conjunction with immunotherapy to enhance efficacy. This has been demonstrated previously in preclinical mouse models of combined PD-1/PD-L1 blockade with MEKi (Liu et al., 2015, Clin Cancer Res An Off J Am Assoc Cancer Res. doi:10.1158/1078-0432.CCR-14-2339, Hu-Lieskovan et al., 2015, Sci Transl Med 7(279):279ra41). Indeed, many of the patients treated currently with immunotherapies also receive kinase inhibitor therapies as distinct treatments.

The loss and gain of function screen described in this Example allowed unbiased interrogation of the currently annotated human kinases for their regulation of cell surface MHC-I. It was mechanistically explored how such kinase regulators could be inhibited for altering surface expression of MHC-I, as a way of validating the screen, understanding the process, and also for extending the findings to functional modulation of a model immunotherapy proof of concept that directly depends on MHC-I presentation. In this case, the MHC was able to be specifically isolated as the sole target of the therapy by use of therapeutic TCR mimic antibodies directed to antigens presented by MHC.

While the effect of the MAPK pathway on MHC-I in vivo was demonstrated herein, the study of certain kinases, such as MEK, to increase MHC-I surface expression and antigen presentation is complex because T-cells and NK cells also rely on similar kinase signaling pathways for activation. Some work has suggested conflicting effects of MEKi on T-cell effector function, and may be dependent on the tumor model evaluated (Ebert et al., 2016, Immunity:1-13, Vella et al., 2014, Cancer Immunol Res:canimm.0181.2013).

The data in this Example provide a mechanistic explanation of how MHC-I is regulated by the MAPK pathway. MHC-I mRNA expression is regulated through upstream enhancer elements, with involvement of the NF-κB transcription factor (Gobin et al., 2003, Blood 101(8):3058-3064, Wolchok and Goodman, 1994, Cytokines 55(January):7-12). MHC-I is also induced by tumor necrosis factor (TNF), interleukin 1 (IL-1), interferon beta, and interferon gamma, which up-regulates HLA-A via the janus kinase (JAK)/STAT pathway (Girdlestone et al., 1993, Proc Natl Acad Sci U S A 90(24):11568-11572, Wolchok and Vilček, 1992, Cytokine 4(6):520-527). The class II, major histocompatibility complex (CIITA) transcription factor can also act on MHC-I gene expression (Gobin et al, 1998, Immunity 9(4):531-541). Interferon gamma has been shown to increase MHC-I and antigen presentation, but thus far has had limited applications for this use (Zaidi and Merlino, 2011, Clin Cancer Res 17(19):6118-6124). MEK has been proposed by others to be a regulator of MHC-I expression. EGFR inhibition can augment MHC-I and MHC-II expression in keratinocytes (Pollack et al., 2011, Clin Cancer Res 17(13):4400-4413). MEK is a negative regulator of HLA-A*02:01 in esophageal and gastric cancer (Mimura et al., 2013, J Immunol 191(12):6261-72). These targets were validated in the screen as important negative regulators of MHC-I and a mechanistic role of the MAP kinase pathway in regulating surface levels of MHC-I was discovered. Interestingly, activating EGFR mutations may contribute to immune escape due to PD-L1 expression. Down-regulation of MHC-I, which was observed from this study, may also contribute to this finding (Akbay et al., 2013, Cancer Discov 3(12):1355-1363). The data in this Example also suggested that using combination therapy of MAP kinase inhibition can be powerful not only as a direct cancer therapy to prevent growth, but also indirectly to promote immunotherapy.

For autoimmune disease, the HLA genes have been shown to be a risk factor for diseases such as ankolysing spondylitis, multiple sclerosis, and other diseases (Fogdell-Hahn et al., 2000, Tissue Antigens 55(2):140-148, Brown et al., 2016, Nat Rev Rheumatol 12(2):81-91, Robert and Kupper, 1999, N Engl J Med 341(10):1817-1828). In addition to up-regulation by certain kinases, down-regulation of MHC-I through new kinase targets was also able to be shown. These targets are not currently addressed by immunosuppressive therapies, which inhibit the effector arm of the immune response with concomitant toxicity.

A requirement of many immunotherapies therapies is the availability of recognizable antigens that are presented on MHC-I. Tumors can down regulate MHC-I to avoid immune system detection of the rare neo-antigens created in tumors by mutations, in addition to up-regulation of inhibitory receptors. By modulating the levels of these limited antigens, improved clinical efficacy could be seen with certain immunotherapies in conjunction with current FDA approved small molecules targeting EGFR and MEK. It was also discovered that the inhibition of the kinase pathways caused a more general up-regulation of the antigen presentation machinery, including TAP (responsible for transporting peptides) and B2M (Beta-2-Microglobulin) (responsible for stabilizing MHC). Many of the recently approved immunotherapies, such as cytotoxic T-lymphocyte-associated protein 4 (CTLA-4) blockade and PD-1 blockade, release the T-cell inhibition promoted by target tumor cells. These immunotherapies provide a promising approach to addressing multiple malignancies, and by rationally combining with targeted small molecule inhibitors, this approach may provide synergistic treatment strategies.

### 6.2. Example 2: Regulation of HLA class I Surface Expression through RET Inhibition

RET is found on chromosome 10 and plays a role in nervous system and kidney development. RET binds to glial cell line-derived neurotrophic factor (GDNF)family of ligands in complex with GDNF receptor alpha (GFRα). Gain of function mutations in RET are often seen in thyroid cancer. Thyroid cancers in follicular thyroid cells are papillary or follicular cancers. Thyroid cancers in parafollicular thyroid cells are medullary or anaplastic cancers.

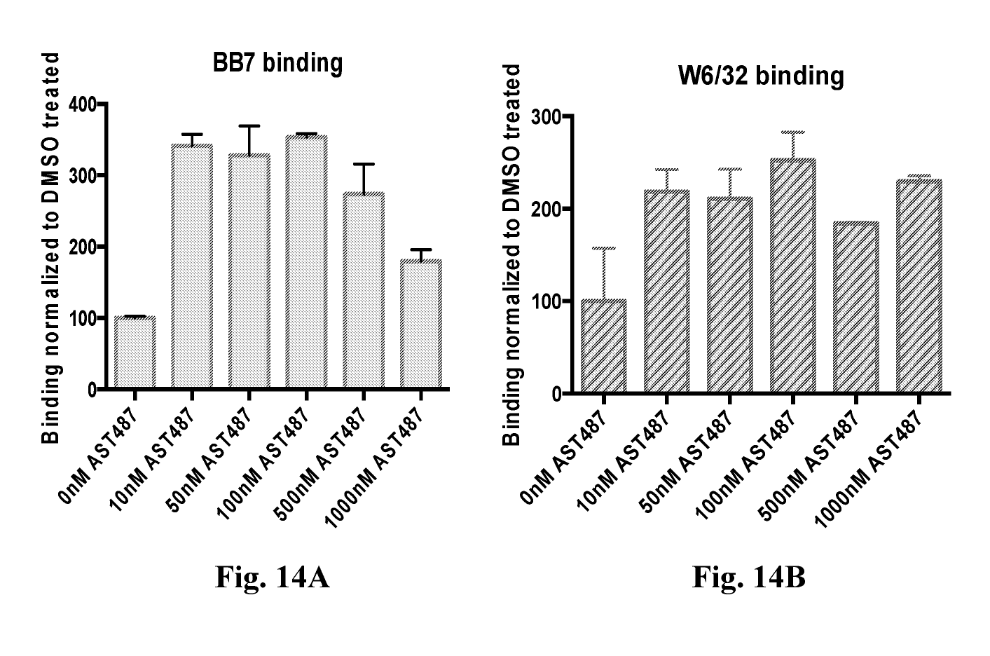

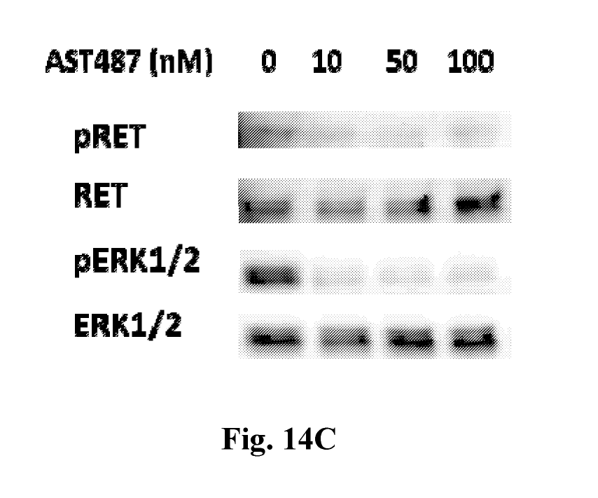

The effect of AST487, a RET inhibitor, on TPC1 (a papillary thyroid cancer cell line) was investigated. TPC1 cells were seeded and treated with different doses of AST487. After 72 hours, cells were harvested and surface HLA-A02 and HLA-ABC were measured through flow cytometry with BB7 and W6/32 staining antibodies, respectively (FIG. 14A-FIG. 14B). After 24 hours of AST487 incubation, cells were lysed and a western blot was performed, which showed a decrease in pRET and pERK with AST487 treatment (FIG. 14C). These data demonstrated that treatment of TPC1 cells with AST487 increased HLA class I surface expression.

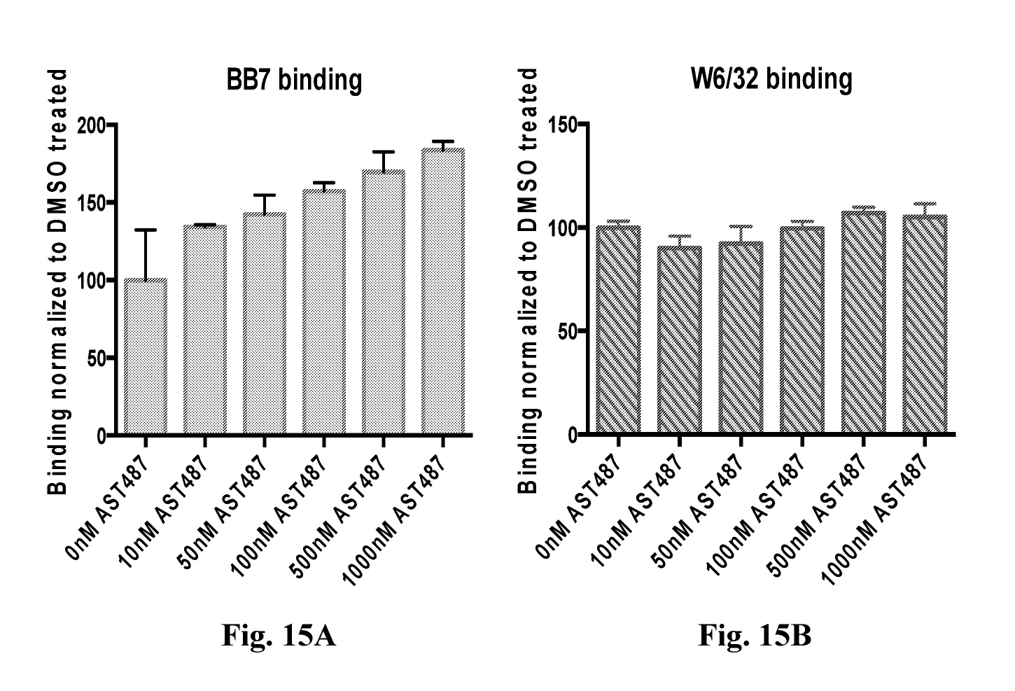

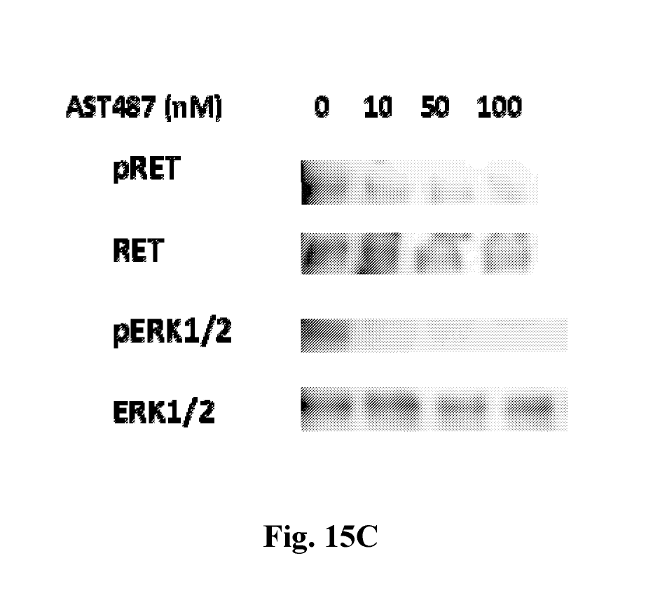

The same analysis was performed in TT cells (a medullary thyroid cancer cell line). TT cells are derived from a medullary thyroid cancer that has a RET point mutation, as opposed to TPC1 cells, which are derived from a papillary thyroid cancer and have a RET/PTC1 fusion. TT cells were seeded and treated with different doses of AST487. After 72 hours, cells were harvested and surface HLA-A02 and HLA-ABC were measured through flow cytometry with BB7 and W6/32 staining antibodies, respectively (FIG. 15A-FIG. 15B). After 24 hours of AST487 incubation, cells were lysed and a western blot was performed (FIG. 15C). These data demonstrated that treatment of TT cells with AST487 upregulated surface HLA-A.

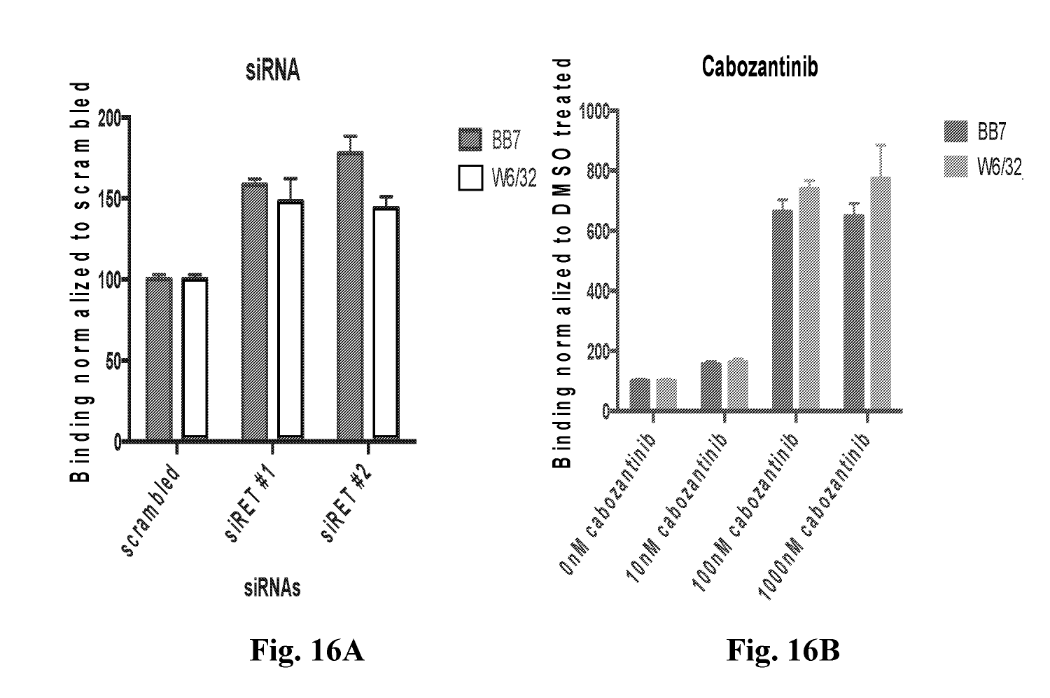

To further validate that RET regulates HLA, siRNAs and another small molecule inhibitor that targeted RET were used. For FIG. 16A, TPC1 cells were treated with siRNAs against a scrambled gene or the RET gene for 96 hours. At that time, surface HLA-A02 and HLA-ABC were measured with BB7 and W6/32 staining antibodies. For FIG. 16B, TPC1 cells were incubated with cabozantinib (a small molecule inhibitor of tyrosine kinases met proto-oncogene (c-MET), vascular endothelial growth factor 2 (VEGF2), KIT proto-oncogene receptor tyrosine kinase (c-KIT), fms-related tyrosine kinase 3 (FLT3) and RET) for 72 hours and surface HLA-A02 and HLA-ABC were measured.

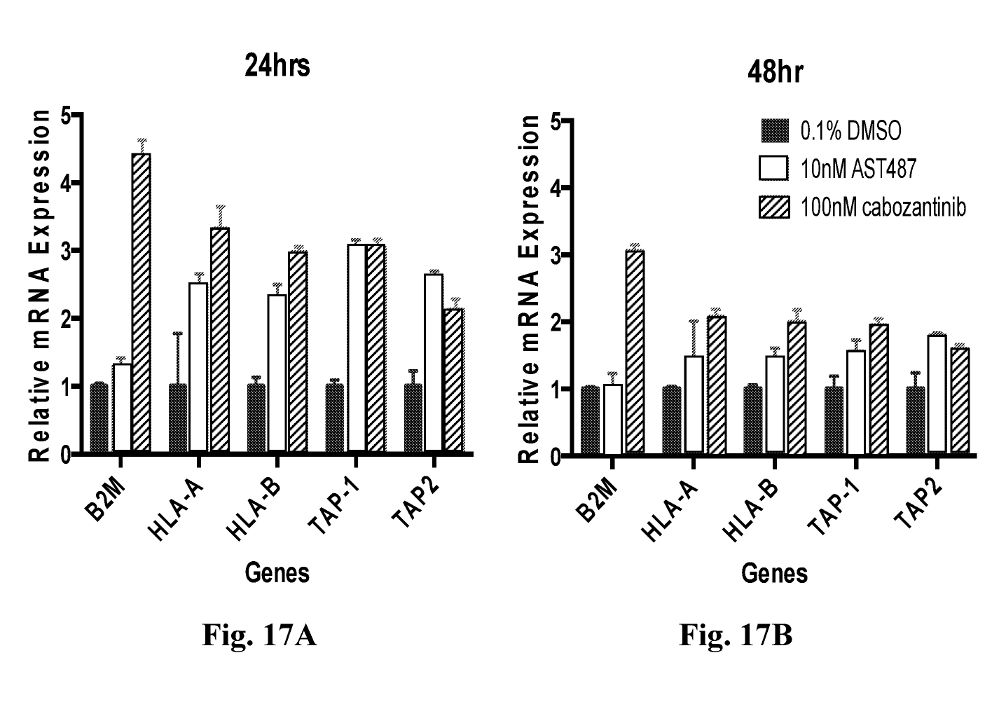

Next, using qPCR, transcript levels of HLA and antigen processing machinery were measured after TPC1 cells were treated with AST487 for 24 (FIG. 17, left panel) or 48 hours (FIG. 17, right panel). Upregulation of mRNA levels for HLA and antigen processing machinery were seen. Regulation of HLA was seen at the transcript level.

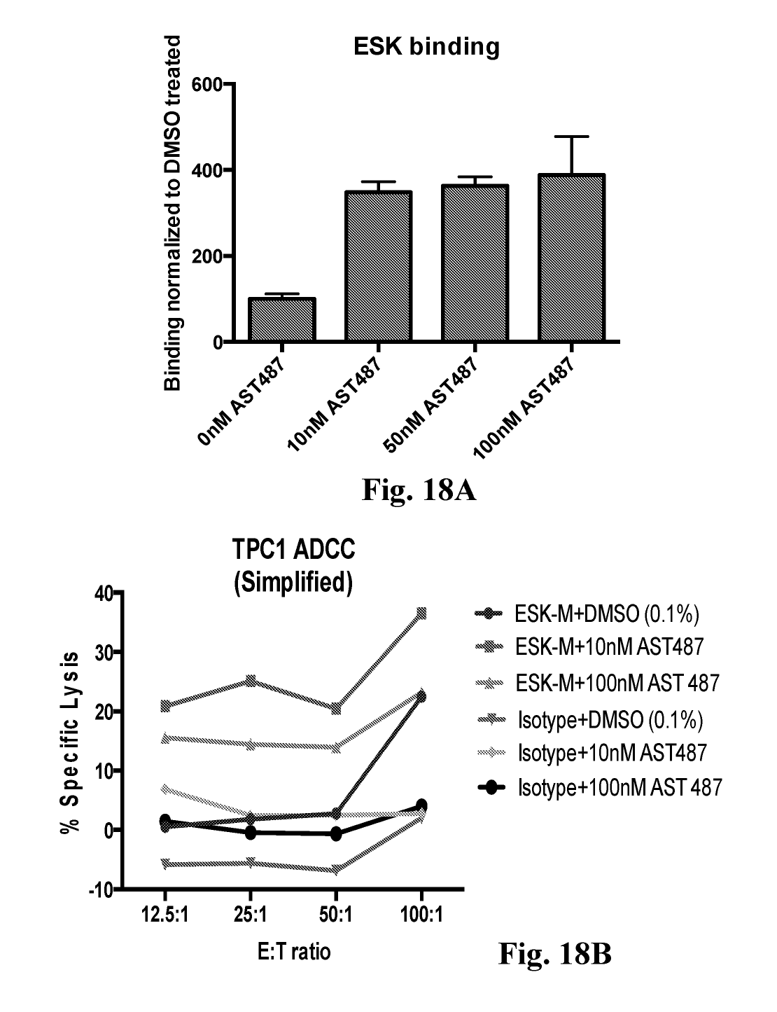

For FIG. 18A, TPC1 cells were treated with different doses of AST487 and binding of ESK (a TCR mimic monoclonal antibody specific for the WT1 RMF peptide/HLA-A02:01 complex) was measured. For FIG. 18B, with increase of ESK binding in vitro, the effect on cytolytic activity of ESK was measured with an antibody-dependent cell-mediated cytotoxicity (ADCC) assay. TPC1 cells were treated with AST487 or DMSO for 72 hours and then incubated with chromium. PBMCs, chromium labeled target cells, and ESK-M (or an isotype) were mixed and incubated for 5 hours. Varying effector to target ratios were used. Afterwards, chromium levels in the media were measured to determine percent specific lysis. These data demonstrated that AST487 increased cytolytic activity of TCRm antibody.

### 6.3. Example 3: RET Inhibition in vivo Increased HLA Class I Antigen Cell Surface Expression

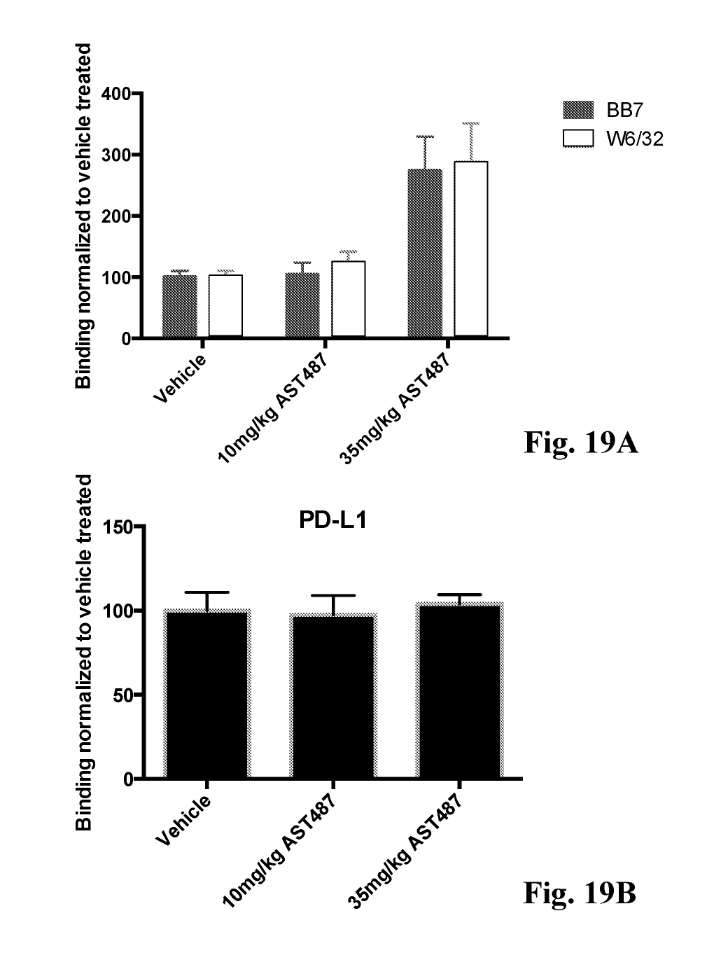

NRG mice (Jackson labs NOD-Rag 1 null IL2rgnull, NOD rag gamma) were injected with TPC1 cells subcutaneously, and then treated 4 weeks later with vehicle (phosphate buffered saline) or with AST487 (a RET inhibitor) at a dose of 10 mg/kg or 35 mg/kg. After 7 days of treatment, tumors were harvested, and tumor cells were assessed for binding to BB7 (a monoclonal antibody specific to HLA-A02) and binding to W6/32 (a monoclonal antibody specific to pan HLA-ABC). Tumor cells were GFP-labeled and were distinguished from stroma and normal cells by gating for GFP in flow cytometry. The results showed that in vivo treatment with AST487 increased HLA-A02 expression (as measured by BB7 binding) and pan HLA-ABC expression (as measured by W6/32 binding) on the surface of the tumor cells (FIG. 19A). One mouse in the vehicle control group was excluded as an outlier with 99% confidence interval as determined by Q test. PD-L1 expression by the tumor was also assessed by measuring binding of anti-PD-L1 antibody. AST487 treatment in vivo did not change expression of PD-L1 by the tumor (FIG. 19B).

### 7. INCORPORATION BY REFERENCE

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A method of treating a cancer in a patient comprising: (i) administering to the patient an inhibitor of the activity of a kinase selected from the group consisting of GRK7 (G Protein-Coupled Receptor Kinase 7), EGFR (Epidermal Growth Factor Receptor), RET (Ret Proto-Oncogene), and BRSK1 (BR Serine/Threonine Kinase 1), and (ii) administering to the patient an immunotherapy that promotes an immune response against the cancer.

2. The method of claim 1, wherein the inhibitor is administered in a subclinical amount.

3. A method of generating a population of antigen-presenting cells for therapeutic administration to a patient having a cancer, comprising culturing antigen-presenting cells that are loaded with or genetically engineered to express one or more immunogenic peptides or proteins derived from one or more antigens of the cancer in the presence of an inhibitor of the activity of a kinase selected from the group consisting of GRK7, EGFR, RET, and BRSK1.

4. A method of treating a cancer in a patient comprising generating a population of antigen-presenting cells according to the method of claim 3, and administering to the patient the population of antigen-presenting cells.

5. The method of any of claims 1-4, wherein the inhibitor is a small molecule inhibitor.

6. The method of any of claims 1-4, wherein the inhibitor is an antibody or an antigen-binding fragment thereof that specifically binds to the kinase.

7. The method of claim 6, wherein the antibody is a monoclonal antibody.

8. The method of any of claims 1-4, wherein the kinase is EGFR and the inhibitor is erlotinib, gefitinib, afatanib, or lapatinib.

9. The method of any of claims 1-4, wherein the kinase is RET and the inhibitor is regorafenib, danusertib, cabozantinib, or AST487 (1-[4-[(4-ethylpiperazin-1-yl)methyl]-3-(trifluoromethyl)phenyl]-3-[4-[6(methylamino)pyrimidin-4-yl]oxyphenyl]urea).

10. A method of treating a cancer in a patient comprising: (i) administering to the patient an activator of the activity of a kinase selected from the group consisting of DDR2 (Discoidin Domain Receptor Tyrosine Kinase 2), CDK7 (Cyclin-Dependent Kinase 7), MINK1 (Misshapen-Like Kinase 1), DAPK3 (Death-Associated Protein Kinase 3), and MAPK3 (Mitogen-Activated Protein Kinase 3), and (ii) administering to the patient an immunotherapy that promotes an immune response against the cancer.

11. The method of claim 10, wherein the activator is administered in a subclinical amount.

12. A method of generating a population of antigen-presenting cells for therapeutic administration to a patient having a cancer, comprising culturing antigen-presenting cells that are loaded with or genetically engineered to express one or more immunogenic peptides or proteins derived from one or more antigens of the cancer in the presence of an activator of the activity of a kinase selected from the group consisting of DDR2, CDK7, MINK1, DAPK3, and MAPK3.

13. A method of treating a cancer in a patient comprising generating a population of antigen-presenting cells according to the method of claim 12, and administering to the patient the population of antigen-presenting cells.

14. The method of any of claims 10-13, wherein the activator is a soluble ligand of the kinase, or a soluble ligand of a receptor that activates the kinase in vivo.

15. The method of any of claims 10-13, wherein the activator is an antibody or an antigen-binding fragment thereof that specifically binds to the kinase.

16. The method of claim 15, wherein the antibody is a monoclonal antibody.

17. The method of any of claims 1-16, wherein the immunotherapy is a vaccine.

18. The method of any of claims 1-16, wherein the immunotherapy is an immune checkpoint blockade.

19. The method of claim 18, wherein the immune checkpoint blockade is an antibody or an antigen-binding fragment thereof that specifically binds to and reduces the activity of an immune checkpoint protein.

20. The method of claim 19, wherein the antibody is a monoclonal antibody.

21. The method of any of claims 18-20, wherein the immune checkpoint blockade inhibits the activity of CTLA-4, PD-1, PD-L1, PD-L2, TIM-3, or LAG-3.

22. The method of any of claims 1-16, wherein the immunotherapy is an adoptive immunotherapy.

23. The method of claim 22, wherein the adoptive immunotherapy is an adoptive T cell therapy.

24. The method of claim 23, wherein the adoptive T cell therapy is TCR (T-Cell Receptor)-engineered T cells.

25. The method of claim 23, wherein the adoptive T cell therapy is CAR (Chimeric Antigen Receptor) T cells, wherein the antigen-binding domain of the CAR specifically binds to an antigen of the cancer.

26. The method of any of claims 1-16, wherein the immunotherapy is a TCR mimic antibody.

27. The method of any of claims 1-16, wherein the immunotherapy is a TCR based construct that encodes a soluble protein comprising the antigen recognition domain of a TCR.

28. The method of any of claims 1-16, wherein the immunotherapy is an interferon, an anti-CD47 antibody, a SIRP alpha antagonist, an HDAC inhibitor, a cytokine, a TLR (Toll-Like Receptor) agonist, or an epigenetic modulator that upregulates the expression of one or more MHCs (Major Histocompatibility Complexes) or upregulates antigen presentation.

29. The method of claim 28, wherein the immunotherapy is an epigenetic modulator that upregulates the expression of one or more MHCs or upregulates antigen presentation that is a hypomethylating agent.

30. The method of claim 29, wherein the immunotherapy is a hypomethylating agent that is azacytidine or decitabine.

31. The method of claim 28, wherein the immunotherapy is an interferon that is interferon alpha or interferon gamma.

32. The method of claim 28, wherein the immunotherapy is a cytokine that is IL2 (Interleukin-2), TNF (Tumor Necrosis Factor), interferon alpha or interferon gamma.

33. The method of claim 28, wherein the immunotherapy is a TLR agonist that is a dsDNA (double-stranded DNA) TLR agonist.

34. The method of claim 28, wherein the immunotherapy is a TLR agonist that is a dsRNA (double-stranded RNA) TLR agonist.

35. The method of claim 34, wherein the immunotherapy is a dsRNA TLR agonist that is polyinosinic-polycytidylic acid (poly(I:C)).

36. The method of any of claims 1-35, wherein the cancer is breast cancer, lung cancer, ovary cancer, stomach cancer, pancreatic cancer, larynx cancer, esophageal cancer, testes cancer, liver cancer, parotid cancer, biliary tract cancer, colon cancer, rectum cancer, cervix cancer, uterus cancer, endometrium cancer, renal cancer, bladder cancer, prostate cancer, thyroid cancer, melanoma, or non-small cell lung cancer.

37. The method of any of claims 1-35, wherein the cancer is lung cancer, thyroid cancer, or melanoma.

38. A method of treating an infection in a patient comprising: (i) administering to the patient an inhibitor of the activity of a kinase selected from the group consisting of GRK7, EGFR, RET, and BRSK1, and (ii) administering to the patient an immunotherapy that promotes an immune response against the infection.

39. The method of claim 38, wherein the inhibitor is administered in a subclinical amount.

40. A method of generating a population of antigen-presenting cells for therapeutic administration to a patient having an infection, comprising culturing antigen-presenting cells that are loaded with or genetically engineered to express one or more immunogenic peptides or proteins derived from one or more antigens of the pathogen causing the infection in the presence of an inhibitor of the activity of a kinase selected from the group consisting of GRK7, EGFR, RET, and BRSK1.

41. A method of treating an infection in a patient comprising generating a population of antigen-presenting cells according to the method of claim 40, and administering to the patient the population of antigen-presenting cells.

42. The method of any of claims 38-41, wherein the inhibitor is a small molecule inhibitor.

43. The method of any of claims 38-41, wherein the inhibitor is an antibody or an antigen-binding fragment thereof that specifically binds to the kinase.

44. The method of claim 43, wherein the antibody is a monoclonal antibody.

45. The method of any of claims 38-41, wherein the kinase is EGFR and the inhibitor is erlotinib, gefitinib, afatanib, or lapatinib.

46. The method of any of claims 38-41, wherein the kinase is RET and the inhibitor is regorafenib, danusertib, cabozantinib, or AST487 (1-[4-[(4-ethylpiperazin-1-yl)methyl]-3-(trifluoromethyl)phenyl]-3-[4-[6-(methylamino)pyrimidin-4-yl]oxyphenyl]urea).

47. A method of treating an infection in a patient comprising: (i) administering to the patient an activator of the activity of a kinase selected from the group consisting of DDR2, CDK7, MINK1, DAPK3, and MAPK3, and (ii) administering to the patient an immunotherapy that promotes an immune response against the infection.

48. The method of claim 47, wherein the activator is administered in a subclinical amount.

49. A method of generating a population of antigen-presenting cells for therapeutic administration to a patient having an infection, comprising culturing antigen-presenting cells that are loaded with or genetically engineered to express one or more immunogenic peptides or proteins derived from one or more antigens of the pathogen causing the infection in the presence of an activator of the activity of a kinase selected from the group consisting of DDR2, CDK7, MINK1, DAPK3, and MAPK3.

50. A method of treating an infection in a patient comprising generating a population of antigen-presenting cells according to the method of claim 49, and administering to the patient the population of antigen-presenting cells.

51. The method of any of claims 47-50, wherein the activator is a soluble ligand of the kinase, or a soluble ligand of a receptor that activates the kinase in vivo.

52. The method of any of claims 47-50, wherein the activator is an antibody or an antigen-binding fragment thereof that specifically binds to the kinase.

53. The method of claim 52, wherein the antibody is a monoclonal antibody.

54. The method of any of claims 38-53, wherein the immunotherapy is a vaccine.

55. The method of any of claims 38-53, wherein the immunotherapy is an immune checkpoint blockade.

56. The method of claim 55, wherein the immune checkpoint blockade is an antibody or an antigen-binding fragment thereof that specifically binds to and reduces the activity of an immune checkpoint protein.

57. The method of claim 56, wherein the antibody is a monoclonal antibody.

58. The method of any of claims 55-57, wherein the immune checkpoint blockade inhibits the activity of CTLA-4, PD-1, PD-L1, PD-L2, TIM-3, or LAG-3.

59. The method of any of claims 38-53, wherein the immunotherapy is an adoptive immunotherapy.

60. The method of claim 59, wherein the adoptive immunotherapy is an adoptive T cell therapy.

61. The method of claim 60, wherein the adoptive T cell therapy is TCR-engineered T cells.

62. The method of claim 60, wherein the adoptive T cell therapy is CART cells, wherein the antigen-binding domain of the CAR specifically binds to an antigen of the cancer.

63. The method of any of claims 38-53, wherein the immunotherapy is a TCR mimic antibody.

64. The method of any of claims 38-53, wherein the immunotherapy is a TCR based construct.

65. The method of any of claims 38-53, wherein the immunotherapy is an interferon, an anti-CD47 antibody, a SIRP alpha antagonist, an HDAC inhibitor, a cytokine, a TLR agonist, or an epigenetic modulator that upregulates the expression of one or more MHCs or upregulates antigen presentation.

66. The method of claim 65, wherein the immunotherapy is an epigenetic modulator that upregulates the expression of one or more MHCs or upregulates antigen presentation that is a hypomethylating agent.

67. The method of claim 66, wherein the immunotherapy is a hypomethylating agent that is azacytidine or decitabine.

68. The method of claim 65, wherein the immunotherapy is an interferon that is interferon alpha or interferon gamma.

69. The method of claim 65, wherein the immunotherapy is a cytokine that is IL2, TNF, interferon alpha or interferon gamma.

70. The method of claim 65, wherein the immunotherapy is a TLR agonist that is a dsDNA (double-stranded DNA) TLR agonist.

71. The method of claim 65, wherein the immunotherapy is a TLR agonist that is a dsRNA (double-stranded RNA) TLR agonist.

72. The method of claim 71, wherein the immunotherapy is a dsRNA TLR agonist that is polyinosinic-polycytidylic acid (poly(I:C)).

73. The method of any of claims 38-72, wherein the infection is an infection with a virus, bacterium, fungus, helminth or protist.

74. The method of claim 73, wherein the infection is an infection with a virus.

75. The method of claim 74, wherein the infection is an infection with herpesvirus.

76. The method of claim 74, wherein the infection is an infection with cytomegalovirus.

77. A method of treating an autoimmune disease in a patient comprising: (i) administering to the patient an activator of the activity of a kinase selected from the group consisting of GRK7, EGFR, RET, and BRSK1, and optionally (ii) administering to the patient an immunosuppressive therapy that suppresses the immune response associated with the autoimmune disease.

78. The method of claim 77, wherein the activator is administered in a subclinical amount.

79. The method of claim 77 or 78, wherein the activator is a soluble ligand of the kinase, or a soluble ligand of a receptor that activates the kinase in vivo.

80. The method of claim 77 or 78, wherein the activator is an antibody or an antigen-binding fragment thereof that specifically binds to the kinase.

81. The method of claim 80, wherein the antibody is a monoclonal antibody.

82. A method of treating an autoimmune disease in a patient comprising: (i) administering to the patient an inhibitor of the activity of a kinase selected from the group consisting of DDR2, CDK7, MINK1, DAPK3, and MAPK3, and optionally (ii) administering to the patient an immunosuppressive therapy that suppresses the immune response associated with the autoimmune disease.

83. The method of claim 82, wherein the inhibitor is administered in a subclinical amount.

84. The method of claim 82 or 83, wherein the inhibitor is a small molecule inhibitor.

85. The method of claim 82 or 83, wherein the inhibitor is an antibody or an antigen-binding fragment thereof that specifically binds to the kinase.

86. The method of claim 85, wherein the antibody is a monoclonal antibody.

87. The method of claim 82 or 83, wherein the kinase is DDR2 and the inhibitor is dasatinib.

88. The method of claim 82 or 83, wherein the kinase is CDK7 and the inhibitor is BS-181 HCl (N5-(6-aminohexyl)-N7-benzyl-3-isopropylpyrazolo[1,5-a]pyrimidine-5,7-diamine hydrochloride).

89. The method of claim 82 or 83, wherein the kinase is DAPK3 and the inhibitor is 324788 ((4Z)-4-(3-Pyridylmethylene)-2-styryl-oxazol-5-one).

90. The method of claim 82 or 83, wherein the kinase is MAPK3 and the inhibitor is ulixertinib.

91. The method of any of claims 77-90, wherein the immunosuppressive therapy is sirolimus, everolimus, rapamycin, one or more steroids, cyclosporine, cyclophosphamide, azathioprine, mercaptopurine, fluorouracil, fludarabine, interferon beta, a TNF decoy receptor, a TNF antibody, methotrexate, a T-cell antibody, an anti-CD20 antibody, a complement inhibitor, an anti-IL6 (Interleukin-6) antibody, an anti-IL2R (Interleukin-2 Receptor) antibody, anti-thymocyte globulin, fingolimod, mycophenolate, or a combination thereof.

92. The method of claim 91, wherein the immunosuppressive therapy is a TNF decoy receptor that is etanercept.

93. The method of claim 91, wherein the immunosuppressive therapy is a TNF antibody that is infliximab.

94. The method of claim 91, wherein the immunosuppressive therapy is a T-cell antibody that is an anti-CD3 antibody.

95. The method of claim 94, wherein the anti-CD3 antibody is OKT3.

96. The method of claim 91, wherein the immunosuppressive therapy is an anti-CD20 antibody that is rituximab.

97. The method of claim 91, wherein the immunosuppressive therapy is a complement inhibitor that is eculizumab.

98. The method of claim 91, wherein the immunosuppressive therapy is an anti-IL2R antibody that is daclizumab.

99. The method of any of claims 77-98, wherein the autoimmune disease is multiple sclerosis, type 1 diabetes , ankylosing spondylitis, or Hashimoto's thyroiditis.

100. A method of treating graft-versus-host disease (GvHD) in a patient comprising:
(i) administering to the patient an activator of the activity of a kinase selected from the group consisting of GRK7, EGFR, RET, and BRSK1, and optionally (ii) administering to the patient an immunosuppressive therapy that suppresses the immune response associated with the GvHD.

101. The method of claim 100, wherein the activator is administered in a subclinical amount.

102. The method of claim 100 or 101, wherein the activator is a soluble ligand of the kinase, or a soluble ligand of a receptor that activates the kinase in vivo.

103. The method of claim 100 or 101, wherein the activator is an antibody or an antigen-binding fragment thereof that specifically binds to the kinase.

104. The method of claim 103, wherein the antibody is a monoclonal antibody.

105. A method of treating a GvHD in a patient comprising: (i) administering to the patient an inhibitor of the activity of a kinase selected from the group consisting of DDR2, CDK7, MINK1, DAPK3, and MAPK3, and optionally (ii) administering to the patient an immunosuppressive therapy that suppresses the immune response associated with the GvHD.

106. The method of claim 105, wherein the inhibitor is administered in a subclinical amount.

107. The method of claim 105 or 106, wherein the inhibitor is a small molecule inhibitor.

108. The method of claim 105 or 106, wherein the inhibitor is an antibody or an antigen-binding fragment thereof that specifically binds to the kinase.

109. The method of claim 108, wherein the antibody is a monoclonal antibody.

110. The method of claim 105 or 106, wherein the kinase is DDR2 and the inhibitor is dasatinib.

111. The method of claim 105 or 106, wherein the kinase is CDK7 and the inhibitor is BS-181 HC1 (N5-(6-aminohexyl)-N7-benzyl-3-isopropylpyrazolo[1,5-a]pyrimidine-5,7-diamine hydrochloride).

112. The method of claim 105 or 106, wherein the kinase is DAPK3 and the inhibitor is 324788 ((4Z)-4-(3-Pyridylmethylene)-2-styryl-oxazol-5-one).

113. The method of claim 105 or 106, wherein the kinase is MAPK3 and the inhibitor is ulixertinib.

114. The method of any of claims 100-113, wherein the immunosuppressive therapy is sirolimus, everolimus, rapamycin, one or more steroids, cyclosporine, cyclophosphamide, azathioprine, mercaptopurine, fluorouracil, fludarabine, interferon beta, a TNF decoy receptor, a TNF antibody, methotrexate, a T-cell antibody, an anti-CD20 antibody, a complement inhibitor, an anti-IL6 antibody, an anti-IL2R antibody, anti-thymocyte globulin, fingolimod, mycophenolate, or a combination thereof

115. The method of claim 114, wherein the immunosuppressive therapy is a TNF decoy receptor that is etanercept.

116. The method of claim 114, wherein the immunosuppressive therapy is a TNF antibody that is infliximab.

117. The method of claim 114, wherein the immunosuppressive therapy is a T-cell antibody that is an anti-CD3 antibody.

118. The method of claim 117, wherein the anti-CD3 antibody is OKT3.

119. The method of claim 114, wherein the immunosuppressive therapy is an anti-CD20 antibody that is rituximab.

120. The method of claim 114, wherein the immunosuppressive therapy is a complement inhibitor that is eculizumab.

121. The method of claim 114, wherein the immunosuppressive therapy is an anti-IL2R antibody that is daclizumab.

122. The method of any of claims 100-121, wherein the autoimmune disease is an acute GvHD.

123. The method of any of claims 100-121, wherein the autoimmune disease is a chronic GvHD.

124. A method of reducing the risk of solid organ transplant rejection in a patient comprising: (i) administering to the patient an activator of the activity of a kinase selected from the group consisting of GRK7, EGFR, RET, and BRSK1, and optionally (ii) administering to the patient an immunosuppressive therapy that suppresses the immune response against the solid organ transplant.

125. A method of reducing the risk of solid organ transplant rejection comprising: (i) administering to the patient an inhibitor of the activity of a kinase selected from the group consisting of DDR2, CDK7, MINK1, DAPK3, and MAPK3, and optionally (ii) administering to the patient an immunosuppressive therapy that suppresses the immune response against the solid organ transplant.

126. The method of any of claims 1-125, wherein the patient is a human patient.
